SEQUENCE LISTING (7 sequences)

SEQ ID NO: 1 - DNA, 1161 bp, Mus musculus
atgtcgtactaccatcaccatcaccatcacgattacgatatcccaacgaccgaaaacctgtattttcagggcgccatgggatccaactcacacaaagatatttctgtgagcatttccgtttcaaattgccagattcaggaaaatgtggatatcagcacagtctatcaaatatttcctgatgaagttctgggttccggacagtttggaattgtttatggaggtaaacatcgtaaaacaggaagagatgtagctattaagattattgacaaattaagatttcctacaaaacaagaaagtcagcttcgtaatgaggttgcaattttacagaaccttcatcaccctggtgttgtaaatttggagtgtatgtttgagacgcctgaaagagtgtttgttgttatggaaaaactccatggagacatgctggagatgatcttgtcaagtgaaaagggctggttgccagaacacataacgaagtttttaattactcagatactagtggctttgcgacatcttcattttaaaaacatcgttcactgtgacctcaagccagaaaatgtgttgctggcatcagccgaccctttccctcaggtgaagctctgtgattttggttttgcccggatcattggagagaagtcttttaggaggtcagtggtgggtaccccagcatacctggcacctgaagttctgaggaacaagggctataaccgctctctggacatgtggtctgttggggtcatcatctatgtgagcctgagtggtaccttcccttttaatgaagatgaagatatccatgatcagatccagaatgcagccttcatgtatccacccaacccgtggaaggagatttctcatgaagccattgatcttatcaataacttgctacaagtgaaaatgagaaaacgctacagtgtggataaaaccttgagtcacccttggctacaggactatcagacctggttagatttacgagagctggaatgcagaattggagaacgctatattacccacgaaagcgatgactccaggtgggaacagtacgcaggcgagcaggggctgcagtacccggcgcacctgatcagtctgagtgctagccacagcgacagtcctgaggctgaagagagagagatgaaagccctcagtgagcgtgtcagcatcctctga

SEQ ID NO: 2 - protein, 386 aa, Mus musculus
MSYYHHHHHHDYDIPTTENLYFQGAMGSNSHKDISVSISVSNCQIQENVDISTVYQIFPDEVLGSGQFGIVYGGKHRKTGRDVAIKIIDKLRFPTKQESQLRNEVAILQNLHHPGVVNLECMFETPERVFVVMEKLHGDMLEMILSSEKGWLPEHITKFLITQILVALRHLHFKNIVHCDLKPENVLLASADPFPQVKLCDFGFARIIGEKSFRRSVVGTPAYLAPEVLRNKGYNRSLDMWSVGVIIYVSLSGTFPFNEDEDIHDQIQNAAFMYPPNPWKEISHEAIDLINNLLQVKMRKRYSVDKTLSHPWLQDYQTWLDLRELECRIGERYITHESDDSRWEQYAGEQGLQYPAHLISLSASHSDSPEAEEREMKALSERVSIL

SEQ ID NO: 3 - protein, 257 aa, Mus musculus
IFPDEVLGSGQFGIVYGGKHRKTGRDVAIKIIDKLRFPTKQESQLRNEVAILQNLHHPGVVNLECMFETPERVFVVMEKLHGDMLEMILSSEKGWLPEHITKFLITQILVALRHLHFKNIVHCDLKPENVLLASADPFPQVKLCDFGFARIIGEKSFRRSVVGTPAYLAPEVLRNKGYNRSLDMWSVGVIIYVSLSGTFPFNEDEDIHDQIQNAAFMYPPNPWKEISHEAIDLINNLLQVKMRKRYSVDKTLSHPWL

SEQ ID NO: 4 - protein, 257 aa, Homo sapiens
IFPDEVLGSGQFGIVYGGKHRKTGRDVAIKIIDKLRFPTKQESQLRNEVAILQNLHHPGVVNLECMFETPERVFVVMEKLHGDMLEMILSSEKGRLPEHITKFLITQILVALRHLHFKNIVHCDLKPENVLLASADPFPQVKLCDFGFARIIGEKSFRRSVVGTPAYLAPEVLRNKGYNRSLDMWSVGVIIYVSLSGTFPFNEDEDIHDQIQNAAFMYPPNPWKEISHEAIDLINNLLQVKMRKRYSVDKTLSHPWL

SEQ ID NO: 5 - protein, 257 aa, Homo sapiens
IFPDEVLGSGQFGVVYGGKHRKTGRDVAVKVIDKLRFPTKQESQLRNEVAILQSLRHPGIVNLECMFETPEKVFVVMEKLHGDMLEMILSSEKGRLPERLTKFLITQILVALRHLHFKNIVHCDLKPENVLLASADPFPQVKLCDFGFARIIGEKSFRRSVVGTPAYLAPEVLLNQGYNRSLDMWSVGVIMYVSLSGTFPFNEDEDINDQIQNAAFMYPASPWSHISAGAIDLINNLLQVKMRKRYSVDKSLSHPWL

SEQ ID NO: 6 - protein, 257 aa, Homo sapiens
IFADEVLGSGGFGIVYGGKHRKTGRDVAIKVIDKMRFPTKQESQLRNEVAILQNLHHPGIVNLECMFETPERVFVVMEKLHGDMLEMILSSEKSRLPERITKFMVTQILVALRNLHFKNIVHCDLKPENVLLASAEPFPQVKLCDFGFARIIGEKSFRRSVVGTPAYLAPEVLRSKGYNRSLDMWSVGVIIYVSLSGTFPFNEDEDINDQIQNAAFMYPPNPWREISGEAIDLINNLLQVKMRKRYSVDKSLSHPWL

SEQ ID NO: 7 - protein, 10 aa, Artificial sequence
  Synthetic sequence Fluorescein labelled glycogen synthase-derived peptide
KKLNRTLSVA

(19) United States
(12) Patent Application Publication
(10) Pub. No.: US 2011/0098325 A1
(43) Pub. Date: Apr. 28, 2011

(54) PYRIDINE BENZAMIDES AND PYRAZINE BENZAMIDES USED AS PKD INHIBITORS

(75) Inventors: Tony Michael Raynham, London (GB); Timothy Robin Hammonds, London (GB); Mark David Charles, London (GB); Grégoire Alexandre Pave, London (GB); Caroline Heather Foxton, London (GB); Wesley Peter Blackaby, Essex (GB); Adrian Philip Stevens, Essex (GB); Chukuemeka Tennyson Ekwuru, Essex (GB)

(73) Assignee: CANCER RESEARCH TECHNOLOGY LIMITED, London (GB)

(21) Appl. No.: 12/520,458

(22) PCT Filed: Dec. 14, 2007
(86) PCT No.: PCT/GB2007/004800

§ 371 Date: Jun. 19, 2009

Related U.S. Application Data
(60) Provisional application No. 60/876,139, filed on Dec. 21, 2006.

(30) Foreign Application Priority Data
Dec. 21, 2006 (GB) 0625659.8

(51) Int. Cl. A61K 31/4418; C12N 9/99; C07D 213/73; A61P 9/00; C07D 213/74; A61P 35/00; A61P 17/06; A61P 17/12; A61P 29/00; A61P 9/04

(52) U.S. Cl. 514/352; 435/184; 546/309

(57) ABSTRACT

The present invention pertains generally to the field of therapeutic compounds, and more specifically to certain pyridine benzamide and pyrazine benzamide compounds (referred to herein as PDBA and PZBA compounds) which, inter alia, inhibit protein kinase D (PKD) (e.g., PKD1, PKD2, PKD3). The present invention also pertains to pharmaceutical compositions comprising such compounds, and the use of such compounds and compositions, both in vitro and in vivo, to inhibit PKD, and in the treatment of diseases and conditions that are mediated by PKD, that are ameliorated by the inhibition of PKD, etc., including proliferative conditions such as cancer, etc.

### RELATED APPLICATIONS

This application is related to: United Kingdom patent application number 0625659.8 filed 21 Dec. 2006 and U.S. patent application No. 60/876,139 filed 21 Dec. 2006; the contents of each of which are incorporated herein by reference in their entirety.

### TECHNICAL FIELD

The present invention pertains generally to the field of therapeutic compounds, and more specifically to certain pyridine benzamide and pyrazine benzamide compounds (referred to herein as PDBA and PZBA compounds) which, inter alia, inhibit protein kinase D (PKD) (e.g., PKD1, PKD2, PKD3). The present invention also pertains to pharmaceutical compositions comprising such compounds, and the use of such compounds and compositions, both in vitro and in vivo, to inhibit PKD, and in the treatment of diseases and conditions that are mediated by PKD, that are ameliorated by the inhibition of PKD, etc., including proliferative conditions such as cancer, etc.

### BACKGROUND

A number of patents and publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

### Protein Kinase D

Protein Kinase D1 (PKD1, also known as Protein Kinase C mu—PKCμ) is the prototypical member of a family of three highly related serine/threonine kinase isoforms, PKD1, PKD2 and PKD3 (formally PKDv). Except where otherwise indicated, a reference to PKD is intended to be a reference to one or more or all of PKD1, PKD2, and PKD3. The PKDs are related to the PKC family by way of C1 domains (Van Lint, 2002), but based on sequence similarities they are now grouped into the calcium calmodulin-dependent kinase (CAMK) family of kinases (see, e.g., Doppler, 2005).

The activity of the PKD family is regulated by at least three different means. Firstly, the PKDs are targets for the actions of the phorbol esters that are known tumour promoters (see, e.g., Van Lint et al., 1995). Phorbol esters regulate the cell localisation and activity of proteins containing conserved DAG-binding cysteine-rich domain (C1 domains). Secondly, the PKDs are activated in a PKC and/or tyrosine kinase dependent manner in response to multiple mitogenic signals including bombesin and PDGF (see, e.g., Zugaza et al., 1996; Matthews et al., 2000b; Storz, et al. 2004). Thirdly, the activity of the PKDs can also be regulated by its interaction with lipids and/or proteins that also regulate its sub-cellular localisation (see, e.g., Wood et al, 2005).

Recent findings have shown that PKD1 is phosphorylated on multiple sites during in vivo activation. Five phosphorylation sites have been identified in PKD1: two sites in the regulatory domain, two in the catalytic domain, and one at the C-terminus. Ser744 and Ser748 (both in the activation loop) play a crucial role in the activation of PKD1. Substitution of these amino acids with alanine completely blocks PKD1 activation, while substitution with glutamic acid (mimicking phosphorylation) causes a constitutive activation. Ser916 (C-terminus) is an autophosphorylation site, not required for activation but rather regulating the conformation of PKD1. Ser203 (regulatory domain) is an autophosphorylation site and is located in the region that interacts with 14-3-3 proteins. Ser255 (in the regulatory domain) is a transphosphorylation site, targeted by PKC or a PKC-activated kinase.

The PKD family is an integral part of a number of signalling cascades that are aberrantly activated during a number of pathological conditions. Activated PKDs are known to be required for a number of cellular processes that have been demonstrated to be suitable points of therapeutic intervention:

### Cancer

The PKDs play a key role in promotion of cell proliferation, invasion, and inhibition of apoptosis, indicating that it is a suitable target for anti-cancer therapeutics. Evidence for these activities comes from the following observations:
Proliferation associated expression of PKD1 and PKD2 has been observed in CML, prostate cancer, small cell lung cancer, and pancreatic carcinoma lines (see, e.g., Mihailovic et al., 2004; Stewart and O'Brian, 2004; Paolucci and Rozengurt, 1999; Guha et al., 2002, 2003).
PKD1 is activated by growth stimuli in both small cell lung cancer (see, e.g., Paolucci & Rozengurt, 1999) and pancreatic cancer cell lines (see, e.g., Guha et al., 2002) contributing to increased colony formation, activation of the MEK/ERK pathway (see, e.g., Guha et al., 2003) and apoptotic blockade (see, e.g., Trauzold et al., 2003).
Inhibition of PKD1 and PKD2 activation by known pharmacological agents (e.g., GF 109203X, U0126) blocks proliferation and colony formation in pancreatic and small cell carcinoma cell lines (see, e.g., Guha et al., 2002, 2003).
The interaction of PKD1 signalling with other transduction pathways (e.g., c-JUN, EGF stimulation of proliferation) is altered in cancer-derived cell lines (see, e.g., Hurd, 2002; Hurd and Rozengurt, 2003).
Mouse skin carcinomas display increased PKD1 expression and over-expression of PKD1 potentiates DNA synthesis and cell proliferation induced by bombesin, vasopressin, and phorbol esters (see, e.g., Zugaza et al., 1997).
In breast cancer, PKD1 is recruited to the leading edge of the cells invading the surrounding tissue forming a complex with actin-binding protein contactin and the focal adhesion protein paxillin (see, e.g., Bowden et al., 1999).

Activation of PKD1 is required for increased adhesion of breast cancer cells to collagen in response to arachidoic acid (see, e.g., Kennett et al., 2004).
Expression of PKD1 correlates with keratinocyte proliferation (see, e.g., Rennecke et al., 1999) and is high in basal dividing cells but low in differentiating cells. Over-expression of PKD1 reduces the sensitivity of several cell types (human and murine) to TNF induced apoptosis (see, e.g., Johannes et al., 1998).
PKD1 phosphorylation of RINI increases RAS/RAF interactions in Cos7 cells; the authors postulate this to be an important inhibition of a negative regulator of a tumourigenic pathway (see, e.g., Wang, 2002).

PKD1 and PKD2 have been shown to selectively phosphorylate HSP27 at serine 82, an event which modulates hsp27 oligomerization and activity. Inhibiting this reaction would potentially be of therapeutic benefit because hsp27 is reported as a survival factor and/or indicator of poor prognosis in prostate, breast and colon cancers. (see, e.g., Doppler, 2005; Gamido, 2003).

Results from an siRNA screen of human kinases has identified PKD2 as a survival kinase (see, e.g., Mackeigan et al., 2005).

Additionally, PKD1 and PKD2 activity is required for cell survival mediated by NF-κB in response to oxidative stress which can be relevant in malignancy especially where DNA damaging agents are being used (see, e.g., Storz & Toker, 2003; Storz et al., 2004a; Storz et al., 2004b). Therefore inhibitors of PKD1 and PKD2 may also be useful as chemo- or radio-potentiating agents.

### Hyperproliferative Skin Disorders

Keratinocytes undergo a distinct pattern of proliferation and differentiation that is essential for the function of the skin as a protective barrier. Defects in the equilibrium between proliferation and differentiation compromise the skin's barrier function and give rise to human diseases such as psoriasis and non-melanoma skin cancer. The identification of protein kinase C (PKC) as a major cellular target for tumor-promoting phorbol esters suggested the involvement of this enzyme in the regulation of keratinocyte proliferation and tumorigenesis; however, results have demonstrated the existence in keratinocytes and other cell types of another diacylglycerol/phorbol ester-responsive protein kinase: protein kinase D1 (PKD1).

Current treatment strategies for hyperproliferative skin disorders are often suboptimal, either because of lack of efficacy or because of contraindications due to deleterious side effects or aesthetic considerations. Thus, small molecule PKD1 inhibitors could be useful for treatment of hyperproliferative skin disorders such as psoriasis, actinic keratosis and nonmelanoma skin cancers (see, e.g., Bollag et al 2004; Ristich, 2006).

### Angiogenesis

Activity of PKD1 is known to be required for Vascular Endothelial Growth Factor (VEGF) stimulated endothelial cell proliferation (see, e.g., Wong and Jin, 2005). VEGF is essential for many angiogenic processes both in normal conditions and in pathological conditions. VEGF rapidly and strongly stimulated PKD1 phosphorylation and activation in endothelial cells via VEGF receptor 2 (VEGFR2). Small interfering RNA knockdown of PKD1 and PKCalpha expression significantly attenuated ERK activation and DNA synthesis in endothelial cells by VEGF. Small interfering RNA knockdown of PKD1 expression significantly attenuates angiogenesis in a matrigel in vivo study (Qin, 2006). Taken together, this demonstrates that VEGF activates PKD1 via the VEGFR2/PLCgamma/PKCalpha pathway and reveals a critical role of PKD1 in angiogenesis, VEGF-induced ERK signalling, and endothelial cell proliferation.

### Inflammation

PKD1 is highly expressed in both T and B lymphocytes, and antigen receptor engagement rapidly stimulates PKD1 activity (see, e.g., Matthews et al., 2000a, 2000b). In T-cells, PKD is rapidly activated and recruited to the plasma membrane (see, e.g., Matthews et al., 2000a). PKD1 residence at the membrane is relatively short, and during the prolonged phase of antigen-receptor activation PKD1 relocates to the cytosol where it remains active for several hours. PKD1 is thus able to transduce a transient signal generated by antigen receptors at the plasma membrane into a sustained signal in the cell interior. As a result, inhibitors of PKD1 could be useful for treatment of inflammatory diseases involving pathological activation of T- and B-cell lymphocytes, neutrophils and Mast cells.

### Heart Failure

In response to acute and chronic stresses, the heart frequently undergoes a remodeling process that is accompanied by myocyte hypertrophy, impaired contractility, and pump failure, often culminating in sudden death. The existence of redundant signaling pathways that trigger heart failure poses challenges for therapeutic intervention. Cardiac remodeling is associated with the activation of a pathological gene program that weakens cardiac performance. Thus, targeting the disease process at the level of gene expression represents a potentially powerful therapeutic approach (see, e.g., Vega et al., 2004; McKinsey and Olson 2005; WO04112763).

PKD1, PKD2, and PKD3 phosphorylates HDAC5 (Huynh Q K, 2006) and this result in HDAC nuclear export. Importantly, small molecule inhibitors that target PKC and PKD1, PKD2, and PKD3, but not CaMK, abolish agonist-mediated nuclear export of HDAC5 cardiac myocytes, which suggests a predominant role for this pathway in the control of HDAC5 in the heart. One point on intervention in this process is via inhibition of Histone DeAcetylases (HDACs). Therefore small molecule PKD1, PKD2, and PKD3 inhibitors could be used to block pathologic cardiac hypertrophy or heart failure.

WO 2003/093297 A2 (Exelixis, Inc.) describes a large number of compounds that apparently modulate protein kinase enzymatic activity and apparently are useful for modulating cellular activities such as proliferation, differentiation, programmed cell death, migration, and chemoinvasion. It appears that some of these compounds may be the following (all of which have a benzyl-amino-acyl group):

| # | Structure | Name | Registry No. |
|---|---|---|---|
| A1 | 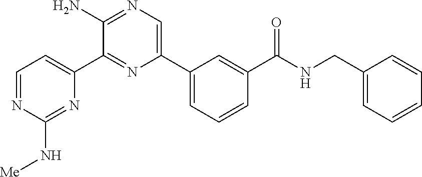 | 3-[5-Amino-6- (2-methylamino- pyrimidin-4-yl)- pyrazin-2-yl]-N- benzyl-benzamide | 625466-02-6 |
| A2 | 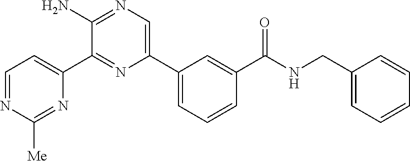 | 3-[5-Amino-6-(2- amino-pyrimidin-4-yl)- pyrazin-2-yl]-N- benzyl-benzamide | 625466-01-5 |
| A3 | 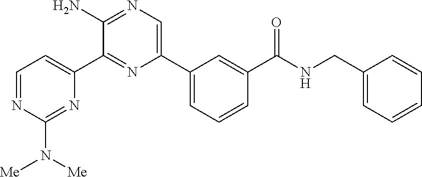 | 3-[5-Amino-6- (2-dimethylamino- pyrimidin-4-yl)- pyrazin-2-yl]-N- benzyl-benzamide | 625465-55-6 |
| A4 | 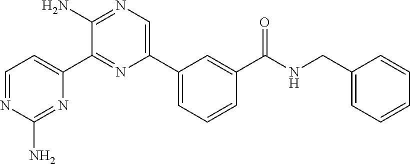 | 3-[5-Amino-6-(2- amino-pyrimidin-4-yl)- pyrazin-2-yl]-N- benzyl-benzamide | 625465-54-5 |

### BRIEF DESCRIPTION OF THE DRAWINGS

FIG. 1 shows the DNA sequence corresponding to murine PKD1.

FIG. 2 shows the amino acid sequence for the murine PKD1 protein used in the biological studies.

FIG. 3 shows the alignment of the kinase domain of murine PKD1 (mPKD1) with those of human PKD1, PKD2, and PKD3 (hPKD1, hPKD2, hPKD3, respectively). Those residues within the ATP binding site are shown in bold, and are completely conserved across the sequences. The kinase domain of murine PKD1 is 99.6%, 91.8% and 93.8% identical to, and 99.7%, 95.4% and 96.5% similar to, human PKD1, PKD2, and PKD3 respectively. The biological data generated in respect of compounds using murine PKD1 are predictive of their activity in respect of any of the human PKD isoforms.

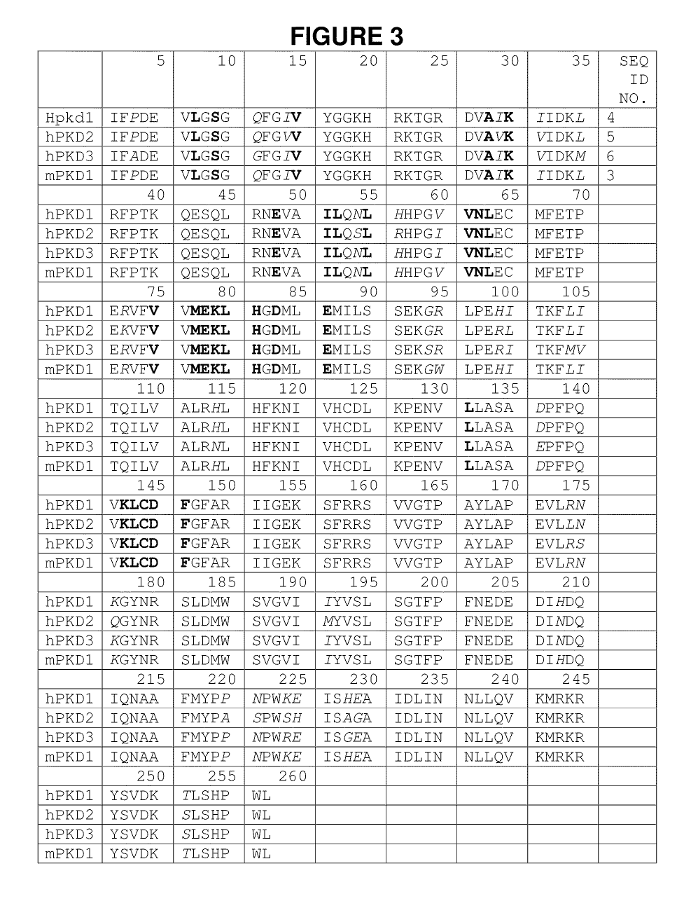

FIG. 4 is a photographic depiction of the western blot analysis of cell lysates of PANC-1 cells which were treated with increasing amounts (1, 10, and 30 μM) of a pyridine benzamide (PDBA), as described below for the Western Blot 916 Assay. Cell lysates were analysed using an anti-human PKD1 Antibody (lower panel) and anti-phospho-human PKD1 (Ser916) Antibody (top panel).

FIG. 5 is a depiction of the quantification of the western blot as shown in FIG. 4. The shown columns represent the % phosphorylation as measured by densitometry of phospho-human PKD1 (Ser916) levels, as described below for the Western Blot 916 Assay. The results were normalised to the measured PKD1 levels and expressed as % of the level of phosphorylation in the PDBu-stimulated control.

FIG. 6 shows a graphic representation of the results obtained in the apoptosis assay, as described below. The depicted lines show the change in viability or induction of apotosis in the presence of a pyridine benzamide (PDBA) compound, as described herein. Cell viability was measured by the MTT assay and induction of apoptosis was measured by the caspase assay after 48 hours. The data are expressed as a % of the level in the corresponding control.

### SUMMARY OF THE INVENTION

One aspect of the invention pertains to certain pyridine benzamide and pyrazine benzamide compounds (referred to herein as PDBA compounds and PZBA compounds), as described herein.

Another aspect of the invention pertains to a composition (e.g., a pharmaceutical composition) comprising a PDBA or PZBA compound, as described herein, and a pharmaceutically acceptable carrier or diluent.

Another aspect of the invention pertains to method of preparing a composition (e.g., a pharmaceutical composition) comprising the step of admixing a PDBA or PZBA compound, as described herein, and a pharmaceutically acceptable carrier or diluent.

Another aspect of the present invention pertains to a method of inhibiting PKD (e.g., PKD1, PKD2, PKD3) in a cell, in vitro or in vivo, comprising contacting the cell with an effective amount of a PDBA or PZBA compound, as described herein.

Another aspect of the present invention pertains to a method of regulating (e.g., inhibiting) cell proliferation (e.g., proliferation of a cell), inhibiting cell cycle progression, promoting apoptosis, or a combination of one or more these, in vitro or in vivo, comprising contacting cells (or the cell) with an effective amount of a PDBA or PZBA compound, as described herein.

Another aspect of the present invention pertains to a method for treatment comprising administering to a subject in need of treatment a therapeutically-effective amount of a PDBA or PZBA compound, as described herein, preferably in the form of a pharmaceutical composition.

Another aspect of the present invention pertains to a PDBA or PZBA compound as described herein for use in a method of treatment of the human or animal body by therapy.

Another aspect of the present invention pertains to use of a PDBA or PZBA compound, as described herein, in the manufacture of a medicament for use in treatment.

In one embodiment, the treatment is treatment of a disease or condition that is mediated by PKD (e.g., PKD1, PKD2, PKD3).

In one embodiment, the treatment is treatment of a disease or condition that is ameliorated by the inhibition of PKD (e.g., PKD1, PKD2, PKD3).

In one embodiment, the treatment is treatment of a proliferative condition.

In one embodiment, the treatment is treatment of cancer.

In one embodiment, the treatment is treatment of a hyperproliferative skin disorder, for example, psoriasis, actinic keratosis, and/or non-melanoma skin cancer.

In one embodiment, the treatment is treatment of a disease or condition that is characterised by inappropriate, excessive, and/or undesirable angiogenesis, for example, macular degeneration, cancer (solid tumours), psoriasis, and obesity.

In one embodiment, the treatment is treatment of an inflammatory disease.

In one embodiment, the treatment is treatment a disease or disorder associated with heart remodelling, myocyte hypertrophy of the heart, impaired contractility of the heart, pump failure of the heart, pathologic cardiac hypertrophy, and/or heart failure.

Another aspect of the present invention pertains to a kit comprising (a) a PDBA or PZBA compound, as described herein, preferably provided as a pharmaceutical composition and in a suitable container and/or with suitable packaging; and (b) instructions for use, for example, written instructions on how to administer the compound.

Another aspect of the present invention pertains to a PDBA or PZBA compound obtainable by a method of synthesis as described herein, or a method comprising a method of synthesis as described herein.

Another aspect of the present invention pertains to a PDBA or PZBA compound obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspect of the invention.

### DETAILED DESCRIPTION OF THE INVENTION

### Compounds

One aspect of the present invention pertains to compound selected from compounds of the following formula (denoted “pyridine benzamide (PDBA) compounds” and pyrazine benzamide (PZBA) compounds”):

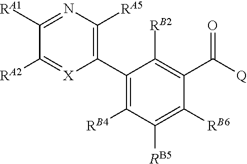

and pharmaceutically acceptable salts, solvates, chemically protected forms, and prodrugs thereof; wherein X, R^{A1}, R^{A2}, R^{A5}, R^{B2}, R^{B4}, R^{B6}, and Q are as defined herein.

For convenience, the compounds may be described as having two core rings: a 6-membered Ring A (a pyridine ring or a pyrazine ring) linked by a single covalent bond to a 6-membered Ring B (a benzene ring), which itself bears a group, —C(═O)Q, attached in a “meta” orientation, as illustrated below:

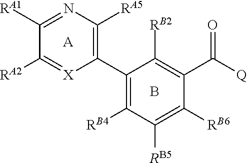

For the avoidance of doubt, it is not intended that Ring A be fused to any other rings; and it is not intended that Ring B be fused to any other rings.

For the avoidance of doubt, it is not intended that Ring A be linked to Ring B, other than by the single covalent bond shown.

For the avoidance of doubt, it is not intended that the group —C(═O)Q be linked to Ring A; and it is not intended that the group —C(═O)Q be linked to Ring B, other than by the single covalent bond shown.

### Provisos

In one or more aspects of the present invention (e.g., compounds, compositions, etc.), the compounds are optionally as defined herein, but with the proviso is that the compound is not:
(B1) N-(3-dimethylamino-propyl)-3-[6-(3-methoxy-phenyl)-pyrazin-2-yl]-benzamide;
(B2) N-(2-dimethylamino-ethyl)-3-[6-(2-methoxy-phenyl)-pyrazin-2-yl]-benzamide;
(B3) N-(2-dimethylamino-ethyl)-3-[6-(3,4,5-trimethoxy-phenyl)-pyrazin-2-yl]-benzamide;
(B4) N-(3-dimethylamino-propyl)-3-[6-(4-hydroxy-phenyl)-pyrazin-2-yl]-benzamide;
(B5) N-(2-dimethylamino-ethyl)-3-[6-(4-hydroxymethyl-phenyl)-pyrazin-2-yl]-benzamide;
(B6) 3-[6-(3-acetylamino-phenyl)-pyrazin-2-yl]-N-(3-dimethylamino-propyl)-benzamide;
(B7) N-(2-dimethylamino-ethyl)-3-[6-(4-hydroxy-3-methoxy-phenyl)-pyrazin-2-yl]-benzamide;
(B8) 3-[6-amino-5-(4-hydroxy-3-methoxy-phenyl)-pyridin-3-yl]-benzamide;
(B9) 3-[6-amino-5-(2-methoxy-phenyl)-pyridin-3-yl]-benzamide; or
(B10) {3-[6-amino-5-(2-methoxy-phenyl)-pyridin-3-yl]-phenyl}-(4-methyl-piperazin-1-yl)-methanone.

The pyrazine benzamide compounds above are illustrated below.

| # | Structure | Name |
|---|---|---|
| B1 | 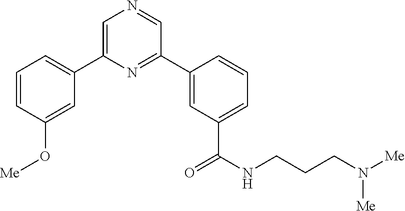 | N-(3-Dimethylamino-propyl)-3-[6-(3- methoxy-phenyl)-pyrazin-2-yl]- benzamide |
| B2 | 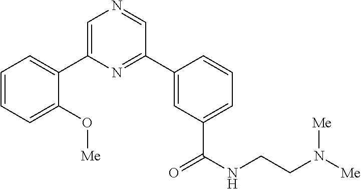 | N-(2-Dimethylamino-ethyl)-3-[6-(2- methoxy-phenyl)-pyrazin-2-yl]- benzamide |
| B3 | 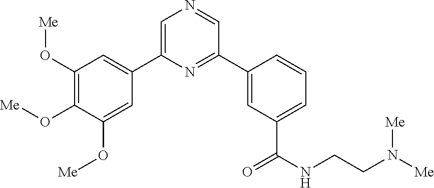 | N-(2-Dimethylamino-ethyl)-3[6- (3,4,5-trimethoxy-phenyl)-pyrazin-2- yl]-benzamide |
| B4 | 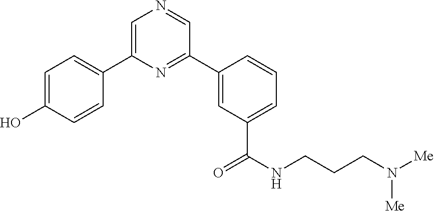 | N-(3-Dimethylamino-propyl)-3-[6-(4- hydroxy-phenyl)-pyrazin-2-yl]- benzamide |
| B5 | 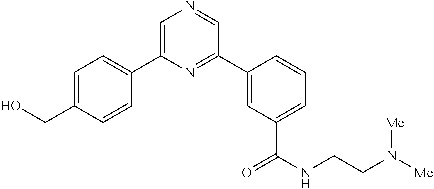 | N-(2-Dimethylamino-ethyl)-3-[6-(4- hydroxymethyl-phenyl)-pyrazin-2- yl]-benzamide |
| B6 | 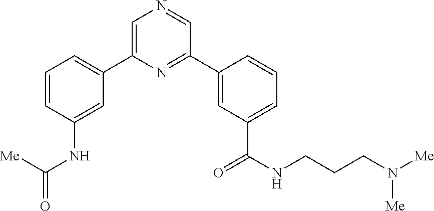 | 3-[6-(3-Acetylamino-phenyl)- pyrazin-2-yl]-N-(3-dimethylamino- propyl)-benzamide |
| B7 | 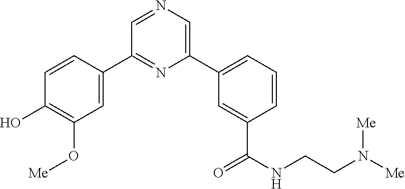 | N-(2-Dimethylamino-ethyl)-3-[6-(4- hydroxy-3-methoxy-phenyl)- pyrazin-2-yl]-benzamide |

The pyridine benzamide compounds above are illustrated below.

| | | |
|---|---|---|
| B8 | 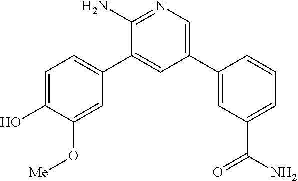 | 3-[6-Amino-5-(4-hydroxy-3- methoxy-phenyl)-pyridin-3-yl]- benzamide |
| B9 | 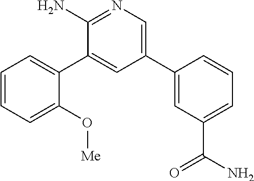 | 3-[6-Amino-5-(2-methoxy-phenyl)- pyridin-3-yl]-benzamide |
| B10 | 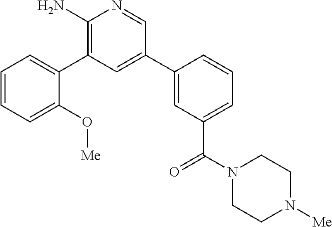 | {3-[6-Amino-5-(2-methoxy-phenyl)- pyridin-3-yl]-phenyl}-(4-methyl- piperazin-1-yl)-methanone |

In one or more aspects of the present invention (e.g., compounds for use in therapy, use of compounds in the manufacture of a medicament, methods of treatment, etc.), the compounds are optionally as defined herein, but without the above proviso.

For example, a reference to a particular group of compounds “without the recited proviso” or “without the recited proviso regarding compounds (B1) to (B10)” (e.g., for use in therapy) is intended to be a reference to the compounds as defined, but wherein the definition no longer includes the indicated proviso. In such cases, it is as if the indicated proviso has been deleted from the definition of compounds, and the definition has been expanded to encompass those compounds which otherwise would have been excluded by the indicated proviso.

### The Group X

The group X is independently C(R^{A3}) or N.

In one embodiment, X is independently C(R^{A3}), and the compounds may conveniently be referred to as “pyridine benzamide compounds” or “PDBA compounds”, as in, for example:

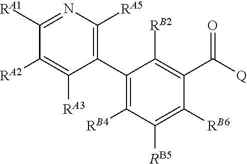

In one embodiment, X is independently N, and the compounds may conveniently be referred to as “pyrazine benzamide compounds” or “PZBA compounds”, as in, for example:

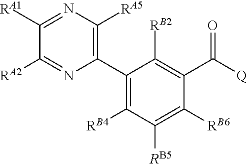

### The Group R^{A1}

The group R^{A1} is independently: —H or —NR^{NA11}R^{NA12};

wherein:
each R^{NA11} is independently —H or R^{Z1};
each R^{NA12} is independently —H or R^{Z1};
   wherein:
each R^{Z1} is independently C₁₋₃alkyl or cyclopropyl;
   and wherein additionally, each —NR^{NA11}R^{NA12} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups.

In one embodiment, R^{A1} is independently: —H or —NR^{NA11}R^{NA12};

wherein:
each R^{NA11} is independently —H or R^{Z1};
each R^{NA12} is independently —H or R^{Z1};
   wherein:
each R^{Z1} is independently C₁₋₃alkyl or cyclopropyl.

In one embodiment, R^{A1} is independently —H, —NH₂, —NHMe, —NMe₂, —NHEt, —NEt₂, or —NMeEt.

In one embodiment, R^{A1} is independently —H or —NH₂.

In one embodiment, R^{A1} is independently —H, as in, for example:

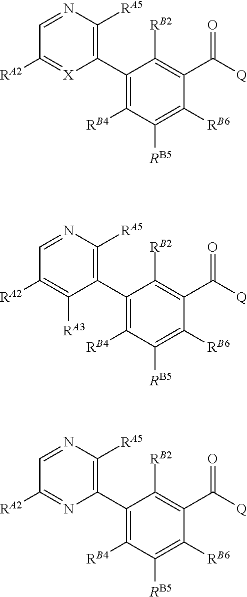

In one embodiment, R^{A1} is independently —NH₂, as in, for example:

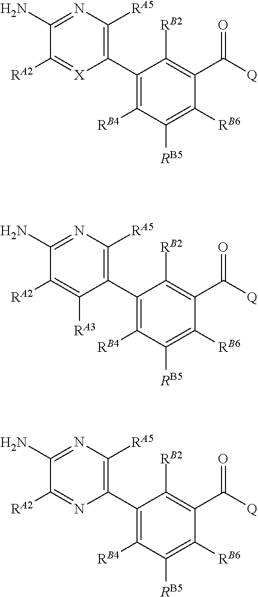

### The Groups R^{A3}, R^{A5}, R^{B2}, R^{B4}, R^{B5}, and R^{B6}

Each of the groups R^{A3}, R^{A5}, R^{B2}, R^{B4}, R^{B5}, and R^{B6}, if present, is independently selected from:
—H, —R^{Z2}, —F, —Cl, —Br, —OH, —OR^{Z2}, —SR^{Z2}, —CF₃, —OCF₃, —CN, —NR^{NZ1}R^{NZ2}, —C(═O)—NR^{NZ1}R^{NZ2}, and —NR^{NZ3}C(═O)R^{Z2};
   wherein:
each R^{NZI} is independently —H or R^{Z2};
each R^{NZ2} is independently —H or R^{Z2};
each R^{NZ3} is independently —H or R^{Z2};
   wherein:
each R^{Z2} is independently C₁₋₃alkyl or cyclopropyl;
   and wherein additionally each —NR^{NZ1}R^{NZ2} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups.

In one embodiment, each of R^{A3}, R^{A5}, R^{B2}, R^{B4}, R^{B5}, and R^{B6}, if present, is independently selected from:
—H, -Me, —F, —Cl, —Br, —OH, —OMe, —SMe, —CF₃, —OCF₃, —CN, —NH₂, —NHMe, —NMe₂, —C(═O)NH₂, —C(═O)NHMe, —C(═O)NMe₂, —NHC(═O)Me and —NMeC(═O)Me.

In one embodiment, each of R^{A3}, R^{A5}, R^{B2}, R^{B4}, R^{B5}, and R^{B6}, if present, is independently selected from:
—H, —F, -Me, and —CF₃.

In one embodiment, each of R^{A3}, R^{A5}, R^{B2}, R^{B4}, R^{B5}, and R^{B6}, if present, is independently —H, as in, for example:

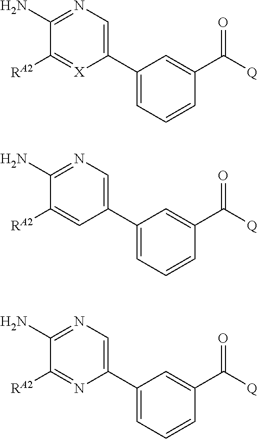

### The Group Q

The group Q is independently —NH₂, —NR^{NQ1}R^{NQ2}, or —W.

In one embodiment, Q is independently —NH₂.

In one embodiment, Q is independently —NR^{NQ1}R^{Q2}.

In one especially preferred embodiment, Q is independently —W.

### The Group Q: —NH₂

In one embodiment, Q is independently —NH₂.

### The Group Q: —NR^{NQ1}R^{NQ2}

In one embodiment, Q is independently —NR^{NQ1}R^{NQ2}, wherein:
R^{NQ1} is independently C₁₋₄alkyl;
R^{NQ2} is independently —H or C₁₋₄alkyl;
and additionally, —NR^{NQ1}R^{NQ2} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups.

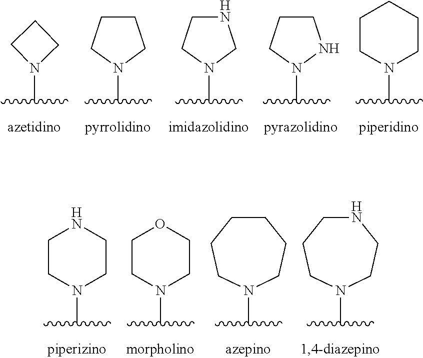

In one embodiment, Q is independently —NR^{NQ1}R^{NQ2}, wherein:
R^{NQ1} is independently C₁₋₄alkyl; and
R^{NQ2} is independently —H or C₁₋₄alkyl.

In one embodiment, Q is independently —NHMe, —NHEt, —NMe₂, or —NEt₂.

### The Group Q: —W

In one especially preferred embodiment, Q is independently —W.

### The Group W

The group W is the following group:

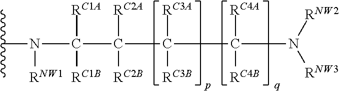

wherein:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H or C₁₋₃alkyl;
each of R^{C3A} and R^{C3B}, if present, is independently —H or C₁₋₃alkyl; and
each of R^{C4A} and R^{C4B}, if present, is independently —H or C₁₋₃alkyl;
   and additionally:
if p is 0 and q is 0, then:
(a1) R^{NW1} and one of R^{NW2} and R^{NW3} may together form:
   —(CH₂)₂— or —(CH₂)₃—; or
(a2) one of R^{C1A} and R^{C1B} and one of R^{NW2} and R^{NW3} may together form:
   —(CH₂)₃— or —(CH₂)₄—; or
(a3) one of R^{C2A} and R^{C2B} and one of R^{NW2} and R^{NW3} may together form:
   —(CH₂)₄— or —(CH₂)₅—;
if p is 1 and q is 0, then:
(b1) R^{NW1} and one of R^{NW2} and R^{NW3} may together form:
   —CH₂— or —(CH₂)₂—; or
(b2) one of R^{C1A} and R^{C1B} and one of R^{NW2} and R^{NW3} may together form:
   —(CH₂)₂— or —(CH₂)₃—; or
(b3) one of R^{C2A} and R^{C2B} and one of R^{NW2} and R^{NW3} may together form:
   —(CH₂)₃— or —(CH₂)₄—;
(b4) one of R^{C3A} and R^{C3B} and one of R^{NW2} and R^{NW3} may together form:
   —(CH₂)₄— or —(CH₂)₅—; and
if p is 1 and q is 1, then:
(c1) R^{NW1} and one of R^{NW2} and R^{NW3} may together form:
   —CH₂—; or
(c2) one of R^{C1A} and R^{C1B} and one of R^{NW2} and R^{NW3} may together form:
   —CH₂— or —(CH₂)₂—; or
(c3) one of R^{C2A} and R^{C2B} and one of R^{NW2} and R^{NW3} may together form:
   —(CH₂)₂— or —(CH₂)₃—; or
(c4) one of R^{C3A} and R^{C3B} and one of R^{NW2} and R^{NW3} may together form:
   —(CH₂)₃— or —(CH₂)₄—; or
(c5) one of R^{C4A} and R^{C4B} and one of R^{NW2} and R^{NW3} may together form:
   —(CH₂)₄— or —(CH₂)₅—.

Examples of W wherein p is 0 and q is 0; and R^{NW1} and one of R^{NW2} and R^{NW3} together form —(CH₂)₂— include the following:

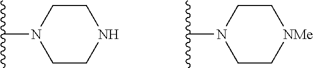

Examples of W wherein p is 1 and q is 0; and one of R^{C1A} and R^{C1B} and one of R^{NW2} and R^{NW3} together form —(CH₂)₂— include the following:

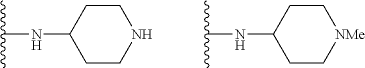

In one embodiment:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H or C₁₋₃alkyl;
each of R^{C3A} and R^{C3B}, if present, is independently —H or C₁₋₃alkyl; and
each of R^{C4A} and R^{C4B}, if present, is independently —H or C₁₋₃alkyl;
   and additionally:
if p is 0 and q is 0, then:
(a1′) R^{NW1} and one of R^{NW2} and R^{NW3} may together form:
   —(CH₂)₂—; or
(a2′) one of R^{C1A} and R^{C1B} and one of R^{NW2} and R^{NW3} may together form:
   —(CH₂)₃—; or
(a3′) one of R^{C2A} and R^{C2B} and one of R^{NW2} and R^{NW3} may together form:
   —(CH₂)₄—;
if p is 1 and q is 0, then:
(b1′) R^{NW1} and one of R^{NW2} and R^{NW3} may together form:
   —CH₂—; or
(b2′) one of R^{C1A} and R^{C1B} and one of R^{NW2} and R^{NW3} may together form:
   —(CH₂)₂—; or
(b3′) one of R^{C2A} and R^{C2B} and one of R^{NW2} and R^{NW3} may together form:
   —(CH₂)₃—;
(b4′) one of R^{C3A} and R^{C2B} and one of R^{NW2} and R^{NW3} may together form:
   —(CH₂)₄—; and
if p is 1 and q is 1, then:
(c2′) one of R^{C1A} and R^{C1B} and one of R^{NW2} and R^{NW3} may together form:
   —CH₂—; or
(c3′) one of R^{C2A} and R^{C2B} and one of R^{NW2} and R^{NW3} may together form:
   —(CH₂)₂—; or
(c4′) one of R^{C3A} and R^{C3B} and one of R^{NW2} and R^{NW3} may together form:
   —(CH₂)₃—; or
(c5′) one of R^{C4A} and R^{C4B} and one of R^{NW2} and R^{NW3} may together form:
   —(CH₂)₄—.

In one embodiment:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H or C₁₋₃alkyl;
each of R^{C3A} and R^{C3B}, if present, is independently —H or C₁₋₃alkyl; and
each of R^{C4A} and R^{C4B}, if present, is independently —H or C₁₋₃alkyl;
   and W may additionally be selected from:

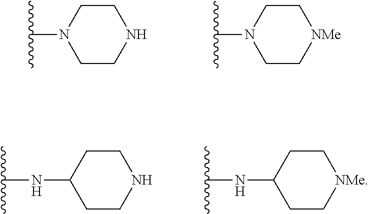

In one embodiment:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR″²R″³ may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H or C₁₋₃alkyl;
each of R^{C3A} and R^{C3B}, if present, is independently —H or C₁₋₃alkyl; and
each of R^{C4A} and R^{C4B}, if present, is independently —H or C₁₋₃alkyl.

In one embodiment:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
each of R^{C1A}, R^{C1B}, R^{C2A}, and R^{C2B} is independently —H or C₁₋₃alkyl;
each of R^{C3A} and R^{C3B}, if present, is independently —H or C₁₋₃alkyl; and
each of R^{C4A} and R^{C4B}, if present, is independently —H or C₁₋₃alkyl.

In one embodiment, R″¹ is independently —H or -Me.

In one embodiment, R″¹ is independently —H.

In one embodiment:
each of R^{C1A}, R^{C1B}, R^{C2A}, and R^{C2B} is independently —H or -Me;
each of R^{C3A} and R^{C3B}, if present, is independently —H or -Me; and
each of R^{C4A} and R^{C4B}, if present, is independently —H or -Me.

In one embodiment:
each of R^{C1A}, R^{C1B}, R^{C2A}, and R^{C2B} is independently —H;
each of R^{C3A} and R^{CSB}, if present, is independently —H; and
each of R^{C4A} and R^{C4B}, if present, is independently —H.

In one embodiment, each of R^{NW2} and R^{NW3} is independently —H or C₁₋₄alkyl.

In one embodiment, each of R^{NW2} and R^{NW3} is independently —H, -Me, or -Et.

In one embodiment, each of R^{NW2} and R^{NW3} is independently C₁₋₄alkyl.

In one embodiment, each of R^{NW2} and R^{NW3} is independently -Me or -Et.

In one embodiment, each of R^{NW2} and R^{NW3} is independently -Me.

In one embodiment, each of R^{NW2} and R^{NW3} is independently —H.

In one embodiment:
p is 0 and q is 0; or
p is 1 and q is 0.

In one embodiment, p is 0 and q is 0, as in, for example:

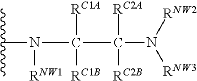

In one embodiment, p is 1 and q is 0, as in, for example:

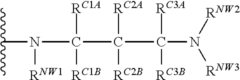

In one embodiment, p is 1 and q is 1, as in, for example:

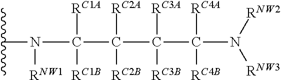

For example, in one embodiment:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H or C₁₋₃alkyl;
each of R^{C3A} and R^{C3B}, if present, is independently —H or C₁₋₃alkyl; and
each of R^{C4A} and R^{C4B}, if present, is independently —H or C₁₋₃alkyl.

Additionally, in one embodiment:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H;
each of R^{C3A} and R^{C3B}, if present, is independently —H; and
each of R^{C4A} and R^{C4B}, if present, is independently —H;
   as in, for example:

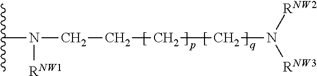

as in, for example:

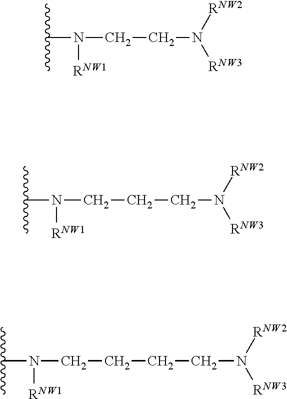

Additionally, in one embodiment:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₃alkyl;
each of R^{C1A}, R^{C1B}, R^{C2A}, and R^{C2B} is independently —H;
each of R^{C3A} and R^{C3B}, if present, is independently —H; and
each of R^{C4A} and R^{C4B}, if present, is independently —H.

Additionally, in one embodiment:
p is 0 and q is 0; or
p is 1 and q is 0; or
p is 1 and q is 1;
R^{NW1} is independently —H, -Me, or -Et;
each of R^{NW2} and R^{NW3} is independently —H, -Me, or -Et;
each of R^{C1A}, R^{C1B}, R^{C2A}, and R^{C2B} is independently —H;
each of R^{C3A} and R^{C3B}, if present, is independently —H; and
each of R^{C4A} and IR^{C4B}, if present, is independently —H.

Additionally, in one embodiment:
p is 0 and q is 0; or
p is 1 and q is 0; or
p is 1 and q is 1;
R^{NW1} is independently —H;
each of R^{NW2} and R^{NW3} is independently —H, -Me, or -Et;
each of R^{C1A}, R^{C1B}, R^{C2A}, and R^{C2B} is independently —H;
each of R^{C3A} and R^{C3B}, if present, is independently —H; and
each of R^{C4A} and R^{C4B}, if present, is independently —H.

Additionally, in one embodiment:
p is 0 and q is 0; or
p is 1 and q is 0; or
p is 1 and q is 1;
R^{NW1} is independently —H;
each of R^{NW2} and R^{NW3} is independently -Me or -Et;
each of R^{C1A}, R^{C1B}, R^{C2A}, and R^{C2B} is independently —H;
each of R^{C3A} and 1R^{C3B}, if present, is independently —H; and
each of R^{C4A} and R^{C4B}, if present, is independently —H.

Additionally, in one embodiment:
p is 0 and q is 0; or
p is 1 and q is 0; or
p is 1 and q is 1;
R^{NW1} is independently —H;
each of R^{NW2} and R^{NW3} is independently -Me;
each of R^{C1A}, R^{C1B}, R^{C2A}, and R^{C2B} is independently —H;
each of R^{C3A} and R^{C3B}, if present, is independently —H; and
each of R^{C4A} and R^{C4B}, if present, is independently —H.

In one embodiment, the group W is the following group:

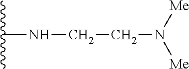

Many of the chemical structures shown herein are silent (or partially silent) with respect to stereoisomeric configuration, and do not indicate any (or all) stereoisomeric configurations. When a chemical structure herein is silent with respect to the stereoisomeric configuration at a position, that chemical structure is intended to depict all possible stereoisomeric configurations at that position, both individually, as if each possible stereoisomeric configuration was individually recited, and also as a mixture (e.g., a racemic mixture) of one or more or all stereoisomers.

### The Group R^{A2}

The group R^{A2} is independently: C₆₋₁₀-carboaryl or C₅₋₁₄heteroaryl; and is independently unsubstituted or substituted.

The group R^{A2} is independently: C₆carboaryl, C₁₀carboaryl, C₅heteroaryl, C₆heteroaryl, C₉heteroaryl, C₁₀heteroaryl, or C₁₃heteroaryl; and is independently unsubstituted or substituted.

In one embodiment, R^{A2} is independently: phenyl, pyridyl, pyrimidinyl, pyrazinyl, pyridazinyl, furanyl, thienyl, pyrrolyl, thiazolyl, oxazolyl, isoxazolyl, isothiazolyl, benzofuranyl, benzo[b]thienyl, indolyl, benzo[1,3]dioxolyl, naphthyl, quinolinyl, isoquinolinyl, quinoxalinyl, indazolyl, 2,3-dihydrobenzo[1,4]dioxinyl, dihydrobenzofuranyl, dibenzofuranyl, and dibenzothienyl; and is independently unsubstituted or substituted.

In one embodiment, R^{A2} is independently: phenyl, pyridyl, pyrimidinyl, pyrazinyl, pyridazinyl, furanyl, thienyl, pyrrolyl, thiazolyl, oxazolyl, isothiazolyl, isoxazolyl, benzofuranyl, benzo[b]thienyl, indolyl, benzo[1,3]dioxolyl, naphthyl, quinolinyl, isoquinolinyl, 2,3-dihydrobenzo[1,4]dioxinyl, dihydrobenzofuranyl, dibenzofuranyl, and dibenzothienyl; and is independently unsubstituted or substituted.

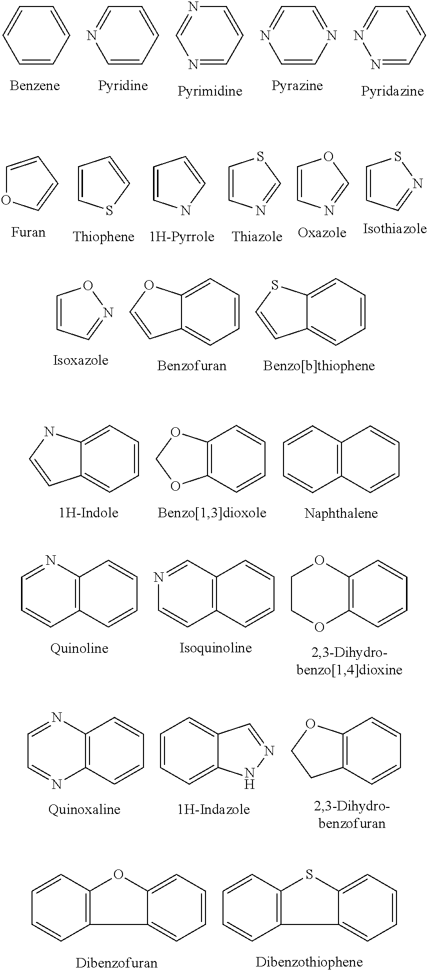

In one embodiment, R^{A2} is independently C₆carboaryl, C₆heteroaryl, C₁₀carboaryl, or C₁₀heteroaryl; and is independently unsubstituted or substituted.

In one embodiment, R^{A2} is independently: phenyl, pyridyl, pyrazinyl, pyrimidinyl, or pyridazinyl, naphthyl, quinolinyl, isoquinolinyl, quinoxalinyl, or indazolyl; and is independently unsubstituted or substituted.

In one embodiment, R^{A2} is independently: phenyl, pyridyl, pyrazinyl, pyrimidinyl, or pyridazinyl, naphthyl, quinolinyl, or isoquinolinyl; and is independently unsubstituted or substituted.

In one embodiment, R^{A2} is independently C₆carboaryl, C₆heteroaryl, C₁₀carboaryl, or C₁₀heteroaryl; and is independently unsubstituted or substituted.

In one embodiment, R^{A2} is independently C₁₀carboaryl or C₁₀heteroaryl; and is independently unsubstituted or substituted.

In one embodiment, R^{A2} is independently C₁₀carboaryl; and is independently unsubstituted or substituted.

In one embodiment, R^{A2} is independently C₁₀heteroaryl; and is independently unsubstituted or substituted.

In one embodiment, R^{A2} is independently naphthyl, quinolinyl, isoquinolinyl, quinoxalinyl, or indazolyl; and is independently unsubstituted or substituted.

In one embodiment, R^{A2} is independently naphthyl, quinolinyl, or isoquinolinyl; and is independently unsubstituted or substituted.

In one embodiment, R^{A2} is independently C₆carboaryl or C₆heteroaryl; and is independently unsubstituted or substituted.

In one embodiment, R^{A2} is independently C₆carboaryl; and is independently unsubstituted or substituted.

In one embodiment, R^{A2} is independently C₆heteroaryl; and is independently unsubstituted or substituted.

In one embodiment, R^{A2} is independently phenyl, pyridyl, or pyrimidinyl; and is independently unsubstituted or substituted.

In one embodiment, R^{A2} is independently phenyl or pyridyl; and is independently unsubstituted or substituted.

In one embodiment, R^{A2} is independently phenyl, and is independently unsubstituted or substituted.

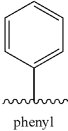

For example, in one embodiment, R^{A2} is independently phenyl, and is independently unsubstituted or substituted, for example, substituted with 1, 2, 3, 4, or 5 substituents, for example, substituted with 1, 2, 3, 4, or 5 substituents as defined below under the heading “Optional Substitutents on R^{A2}”, for example, 1, 2, or 3 substituents independently selected from —OH and —OR, where R is independently saturated aliphatic C₁₋₄alkyl.

In one embodiment, R^{A2} is independently pyridyl, and is independently unsubstituted or substituted.

In one embodiment, R^{A2} is independently 2-pyridyl, and is independently unsubstituted or substituted.

In one embodiment, R^{A2} is independently 3-pyridyl, and is independently unsubstituted or substituted.

In one embodiment, R^{A2} is independently 4-pyridyl, and is independently unsubstituted or substituted.

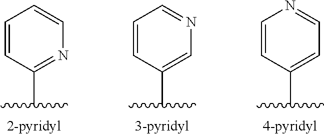

For example, in one embodiment, R^{A2} is independently pyridyl (e.g., 2-pyridyl, 3-pyridyl, 4-pyridyl), and is independently unsubstituted or substituted, for example, substituted with 1, 2, 3, or 4 substituents, for example, substituted with 1, 2, 3, or 4 substituents as defined below under the heading “Optional Substitutents on R^{A2}”, for example, 1 or 2 substituents independently selected from —OH and —OR, where R is independently saturated aliphatic C₁₋₄alkyl.

In one embodiment, R^{A2} is independently pyrimidinyl, and is independently unsubstituted or substituted.

In one embodiment, R^{A2} is independently 4-pyrimidinyl, and is independently unsubstituted or substituted.

In one embodiment, R^{A2} is independently 5-pyrimidinyl, and is independently unsubstituted or substituted.

In one embodiment, R^{A2} is independently 2-pyrimidinyl, and is independently unsubstituted or substituted.

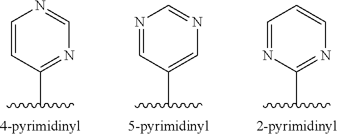

For example, in one embodiment, R^{A2} is independently pyrimidinyl (e.g., 4-pyrimdinyl, 5-pyrimidinyl, 2-pyrimidinyl), and is independently unsubstituted or substituted, for example, substituted with 1, 2 or 3 substituents, for example, substituted with 1, 2, or 3 substituents as defined below under the heading “Optional Substitutents on R^{A2}”, for example, 1 or 2 substituents independently selected from —OH and —OR, where R is independently saturated aliphatic C₁₋₄alkyl.

In one embodiment, R^{A2} is independently naphthyl, and is independently unsubstituted or substituted.

In one embodiment, R^{A2} is independently 1-naphthyl, and is independently unsubstituted or substituted.

In one embodiment, R^{A2} is independently 2-naphthyl, and is independently unsubstituted or substituted.

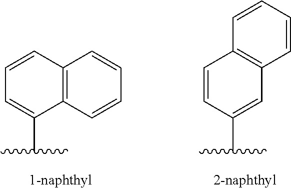

For example, in one embodiment, R^{A2} is independently naphthyl (e.g., 1-naphthyl, 2-naphthyl), and is independently unsubstituted or substituted, for example, substituted with 1, 2, 3, 4, 5, 6, or 7 substituents, for example, substituted with 1, 2, 3, 4, 5, 6, or 7 substituents as defined below under the heading “Optional Substitutents on R^{A2}”, for example, 1, 2, or 3 substituents independently selected from —OH and —OR, where R is independently saturated aliphatic C₁₋₄alkyl.

In one embodiment, R^{A2} is independently quinolinyl or isoquinolinyl, and is independently unsubstituted or substituted.

In one embodiment, R^{A2} is independently quinolinyl, and is independently unsubstituted or substituted.

In one embodiment, R^{A2} is independently 3-quinolinyl, 5-quinolinyl, or 8-quinolinyl, and is independently unsubstituted or substituted.

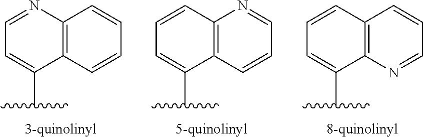

For example, in one embodiment, R^{A2} is independently quinolinyl (e.g., 3-quinolinyl, 5-quinolinyl, 8-quinolinyl), and is independently unsubstituted or substituted, for example, substituted with 1, 2, 3, 4, 5, or 6 substituents, for example, substituted with 1, 2, 3, 4, 5, or 6 substituents as defined below under the heading “Optional Substitutents on R^{A2}”, for example, 1, 2, or 3 substituents independently selected from —OH and —OR, where R is independently saturated aliphatic C₁₋₄alkyl.

In one embodiment, R^{A2} is independently isoquinolinyl, and is independently unsubstituted or substituted.

In one embodiment, R^{A2} is independently 4-isoquinolinyl or 5-isoquinolinyl, and is independently unsubstituted or substituted.

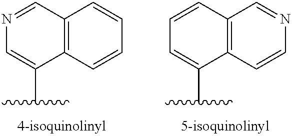

For example, in one embodiment, R^{A2} is independently isoquinolinyl (e.g., 4-isoquinolinyl, 5-isoquinolinyl), and is independently unsubstituted or substituted, for example, substituted with 1, 2, 3, 4, 5, or 6 substituents, for example, substituted with 1, 2, 3, 4, 5, or 6 substituents as defined below under the heading “Optional Substitutents on R^{A2}”, for example, 1, 2, or 3 substituents independently selected from —OH and —OR, where R is independently saturated aliphatic C₁₋₄alkyl.

In one embodiment, R^{A2} is as defined herein, with the proviso that it is not 2-pyrimidinyl or substituted 2-pyrimidinyl.

In one embodiment, R^{A2} is as defined herein, with the proviso that it is not pyrimidinyl or substituted pyrimidinyl.

### Optional Substitutents on R^{A2}

As discussed above, the group R^{A2} is, for example, C₆₋₁₀carboaryl or C₅₋₁₄heteroaryl, and is independently unsubstituted or substituted, for example, substituted with one or more (e.g., 1, 2, etc.) substituents.

For example, in one embodiment, R^{A2} is independently phenyl, and is independently unsubstituted or substituted, for example, substituted with 1, 2, 3, 4, or 5 substituents.

For example, in one embodiment, R^{A2} is independently pyridyl, and is independently unsubstituted or substituted, for example, substituted with 1, 2, 3, or 4 substituents.

For example, in one embodiment, R^{A2} is independently pyrimidinyl, and is independently unsubstituted or substituted, for example, substituted with 1, 2 or 3 substituents.

For example, in one embodiment, R^{A2} is independently naphthyl, and is independently unsubstituted or substituted, for example, substituted with 1, 2, 3, 4, 5, 6, or 7 substituents.

Substituents, if present, may be on a ring carbon atom or a ring heteroatom. For example, when a heteroaryl group includes —NH— in the aromatic ring (e.g., as in pyrrolyl, imidazolyl, pyrazolyl), this group may be N-substituted, for example N—(C₁₋₃alkyl)-substituted, for example N-(methyl)-substituted, as in, for example, N-methyl-pyrazolyl.

In one embodiment, each substituent (e.g., each optional substituent on R^{A2}) is independently selected from:
(H-1) —C(═O)OH;
(H-2) —C(═O)OR^{a};
(H-3) —C(═O)NH₂, —C(═O)NHR^{a}, —C(═O)NR^{a}R^{a}, —C(═O)NR^{b}R^{c};
(H-4) —C(═O)R^{a};
(H-5) —F, —Cl, —Br, —I;
(H-6) —CN;
(H-7) —NO₂;
(H-8) —OH;
(H-9) —OR^{a};
(H-10) —SH;
(H-11) —SR^{a};
(H-12) —OC(═O)R^{a};
(H-13) —OC(═O)NH₂, —OC(═O)NHR^{a}, —OC(═O)NR^{a}R^{a}, —OC(═O)NR^{b}R^{c};
(H-14) —NH₂, —NHR^{a}, —NR^{a}R^{a}, —NR^{b}R^{c};
(H-15) —NHC(═O)R^{a}; —NR^{a}C(═O)R^{a};
(H-16) —NHC(═O)NH₂, —NHC(═O)NHR^{a}, —NHC(═O)NR^{a}R^{a}, —NHC(═O)NR^{b}R^{c}, —NR^{a}C(═O)NH₂, —NR^{a}C(═O)NHR^{a}, —NR^{a}C(═O)NR^{a}R^{a}, —NR^{a}C(═O)NR^{b}R^{c};
(H-17) —NHSO₂R^{a}, —NR^{a}SO₂R^{a};
(H-21) —SO₂R^{a};
(H-22) —OSO₂R^{a};
(H-23) —SO₂NH₂, —SO₂NHR^{a}, —SO₂NR^{a}R^{a}, —SO₂NR^{b}R^{C};
(H-24) ═O; and
(H-25) —R^{d};
   wherein R^{d} and each R^{a} is independently selected from:
(C-1) C₁₋₇alkyl;
(C-2) C₂₋₇alkenyl;
(C-3) C₂₋₇alkynyl;
(C-4) C₃₋₇cycloalkyl;
(C-5) C₃₋₇cycloalkenyl;
(C-6) C₃₋₁₄heterocyclyl,
(C-7) C₆₋₁₄-carboaryl,
(C-8) C₅₋₁₄heteroaryl,
(C-9) C₃₋₇cycloalkyl-C₁₋₇alkylenyl,
(C-10) C₃₋₁₄heterocyclyl-C₁₋₇alkylenyl,
(C-11) C₆₋₁₄-carboaryl-C₁₋₇alkylenyl, and
(C-12) C₅₋₁₄heteroaryl-C₁₋₇alkylenyl;
   wherein each C₁₋₇alkyl, C₂₋₇alkenyl, C₂₋₇alkynyl, C₃₋₇cycloalkyl, C₃₋₇cycloalkenyl, C₃₋₁₄heterocyclyl, C₆₋₁₄-carboaryl, and C₅₋₁₄heteroaryl is independently unsubstituted or substituted with one or more (e.g., 1, 2, etc.) substituents selected from (H-1) through (H-25);
   and wherein R^{b} and R^{c} taken together with the nitrogen atom to which they are attached form a ring having from 3 to 7 ring atoms.

In one embodiment, each substituent (e.g., each optional substituent on R^{A2}) is independently selected from:
(H-1) —C(═O)OH;
(H-2) —C(═O)OR^{a};
(H-3) —C(═O)NH₂, —C(═O)NHR^{a}, —C(═O)NR^{a}R^{a}, —C(═O)NR^{b}R^{c};
(H-4) —C(═O)R^{a};
(H-5) —F, —Cl, —Br, —I;
(H-6) —CN;
(H-8) —OH;
(H-9) —OR^{a};
(H-11) —SR^{a};
(H-14) —NH₂, —NHR^{a}, —NR^{a}R^{a}, —NR^{b}R^{C};
(H-15) —NHC(═O)R^{a}; —NR^{a}C(═O)R^{a};
(H-21) —SO₂R^{a};
(H-25) —R^{d};
   wherein R^{d} and each R^{a} is independently selected from:
(C-1) C₁₋₇alkyl;
(C-6) C₃₋₁₄heterocyclyl,
(C-7) C₆₋₁₄-carboaryl,
(C-8) C₅₋₁₄heteroaryl,
   wherein each C₁₋₇alkyl, C₃₋₁₄heterocyclyl, C₆₋₁₄-carboaryl, and C₅₋₁₄heteroaryl is independently unsubstituted or substituted with one or more (e.g., 1, 2, etc.) substituents selected from (H-1) through (H-25);
   and wherein R^{b} and R^{C} taken together with the nitrogen atom to which they are attached form a ring having from 3 to 7 ring atoms.

In one embodiment, each substituent (e.g., each optional substituent on R^{A2}) is independently selected from:
—F, —Cl, —Br, —I,
—CN, —CH₂CN,
—R^{aa}, —CF₃,
—Ph, —CH₂Ph, thienyl,
—OH, —R^{L}—OH, —R^{L}—OR^{aa},
—OR^{aa}, —OCF₃,
—OPh, —OCH₂Ph,
—O—R^{L}—OH, —O—R^{L}—OR^{aa},
—SR^{aa}, —SPh,
—SO₂R^{aa}, SO₂Ph,
—NHSO₂R^{aa}, NHSO₂Ph,
—NH₂, —NHR^{aa}, —N(R^{aa})₂,
—NHPh, —NHCH₂Ph,
—NH—R^{L}—NH₂, —NH—R^{L}—NHR^{aa}, —NH—R^{L}—N(R^{aa})₂,
—C(═O)NH₂, —C(═O)NHR^{aa}, —C(═O)N(R^{aa})₂,
—C(═O)NHPh, —C(═O)NHCH₂Ph,
—NHC(═O)R^{aa},
—NHC(═O)Ph, —NHC(═O)CH₂Ph,
—C(═O)OH, —C(═O)OR^{aa},
—C(═O)OPh, and —C(═O)OCH₂Ph,
—C(═O)Ph;
wherein:
each Ph is independently phenyl, optionally substituted with 1 to 4 groups selected from:
—F, —Cl, —Br, —I,
—CN,
—R^{aa}, —CF₃,
—OH, —OR^{aa},
—O—R^{L}—OH, —O—R^{L}—OR^{aa},
—OCF₃,
—NH₂, —NHR^{aa}, —N(R^{aa})₂,
—C(═O)NH₂, —C(═O)NHR^{aa}, —C(═O)N(R^{aa})₂,
—NHC(═O)R^{aa},
and wherein:
each R^{aa} is independently C₁₋₄alkyl;
additionally, for each —N(R^{aa})₂, two R^{aa} groups, taken together with the nitrogen atom to which they are attached, may form a non-aromatic heterocyclic ring having from 4 to 7 ring atoms, optionally substituted with one or more C₁₋₃alkyl groups; and
each R^{L} is independently C₁₋₄alkylenyl (e.g., —(CH₂)_{z}—, wherein z is 1, 2, 3, or 4).

In one embodiment, each substituent (e.g., each optional substituent on R^{A2}) is independently selected from:
—F, —Cl, —Br, —I,
—CN,
—R^{aa}, —CF₃,
—Ph, —CH₂Ph, thienyl,
—OH,
—OR^{aa}, —OCF₃,
—OPh, —OCH₂Ph,
—O—R^{L}—OH, —O—R^{L}—OR^{aa},
—SR^{aa}, —SPh,
—SO₂R^{aa}, SO₂Ph,
—NHSO₂R^{aa}, NHSO₂Ph,
—NH₂, —NHR^{aa}, —N(R^{aa})₂,
—NHPh, —NHCH₂Ph,
—NH—R^{L}—NH₂, —NH—R^{L}—NHR^{aa}, —NH—R^{L}—N(R^{aa})₂,
—C(═O)NH₂, —C(═O)NHR^{aa}, —C(═O)N(R^{aa})₂,
—C(═O)NHPh, —C(═O)NHCH₂Ph,
—NHC(═O)R^{aa},
—NHC(═O)Ph, —NHC(═O)CH₂Ph,
—C(═O)OH, —C(═O)OR^{aa},
—C(═O)OPh, and —C(═O)OCH₂Ph,
—C(═O)Ph;
wherein:
each Ph is independently phenyl, optionally substituted with 1 to 4 groups selected from:
—F, —Cl, —Br, —I,
—CN,
—R^{aa}, —CF₃,
—OH, —OR^{aa},
—O—R^{L}—OH, —O—R^{L}—OR^{aa},
—OCF₃,
—NH₂, —NHR^{aa}, —N(R^{aa})₂,
—C(═O)NH₂, —C(═O)NHR^{aa}, —C(═O)N(R^{aa})₂,
—NHC(═O)R^{aa},
and wherein:
each R^{aa} is independently C₁₋₄alkyl;
additionally, for each —N(R^{aa})₂, two R^{aa} groups, taken together with the nitrogen atom to which they are attached, may form a non-aromatic heterocyclic ring having from 4 to 7 ring atoms, optionally substituted with one or more C₁₋₃alkyl groups; and
each R^{L} is independently C₁₋₄alkylenyl (e.g., —(CH₂)_{z}—, wherein z is 1, 2, 3, or 4).

In one embodiment, the substituents are independently selected from those substituents exemplified under the heading “Some Preferred Embodiments.”

### Combinations

Each and every plausible and compatible combination of the embodiments described herein is explicitly disclosed herein, as if each such combination was specifically and individually recited.

(A) For example, in one embodiment:
X is independently C(R^{A3}) or N;
R^{A1} is independently —H or —NH₂;
R^{A2} is as defined herein;
each of R^{A3}, R^{A5}, R^{B2}, R^{B4}, R^{B5}, and R^{B6}, if present, is independently —H; and
Q is W.
   (B) For example, in one embodiment:
X is independently C(R^{A3});
R^{A1} is independently —H or —NH₂;
R^{A2} is as defined herein;
each of R^{A3}, R^{A5}, R^{B2}, R^{B4}, R^{B5}, and R^{B6}, if present, is independently —H; and
Q is W.
   (C) For example, in one embodiment:
X is independently C(R^{A3});
R^{A1} is independently —H;
R^{A2} is as defined herein;
each of R^{A3}, R^{A5}, R^{B2}, R^{B4}, R^{B5}, and R^{B6}, if present, is independently —H; and
Q is W;
   as in, for example:

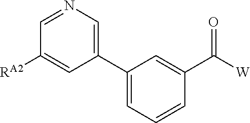

(D) For example, in one embodiment:
X is independently C(R^{A3});
R^{A1} is independently —NH₂;
R^{A2} is as defined herein;
each of R^{A3}, R^{A5}, R^{B2}, R^{B4}, R^{B5}, and R^{B6}, if present, is independently —H; and
Q is W;
   as in, for example:

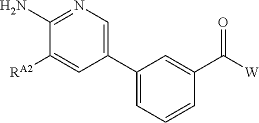

(E) For example, in one embodiment:
X is independently C(R^{A3});
R^{A1} is independently —NH₂;
R^{A2} is independently phenyl, and is independently unsubstituted or substituted;
each of R^{A3}, R^{A5}, R^{B2}, R^{B4}, R^{B5}, and R^{B6}, if present, is independently —H; and
Q is W.
   (F) For example, in one embodiment:
X is independently C(R^{A3});
R^{A1} is independently —NH₂;
R^{A2} is independently pyridyl, and is independently unsubstituted or substituted;
each of R^{A3}, R^{A5}, R^{B2}, R^{B4}, R^{B5}, and R^{B6}, if present, is independently —H; and
Q is W.
   (G) For example, in one embodiment:
X is independently C(R^{A3});
R^{A1} is independently —NH₂;
R^{A2} is independently pyrimidinyl, and is independently unsubstituted or substituted;
each of R^{A3}, R^{A5}, R^{B2}, R^{B4}, R^{B5}, and R^{B6}, if present, is independently —H; and
Q is W.
   (H) For example, in one embodiment:
X is independently C(R^{A3});
R^{A1} is independently —NH₂;
R^{A2} is independently naphthyl, and is independently unsubstituted or substituted;
each of R^{A3}, R^{A5}, R^{B2}, R^{B4}, R^{B5}, and R^{B6}, if present, is independently —H; and
Q is W.
   (I) For example, in one embodiment:
X is independently N;
R^{A2} is independently —H or —NH₂;
R^{A2} is as defined herein;
each of R^{A3}, R^{A5}, R^{B2}, R^{B4}, R^{B5}, and R^{B6}, if present, is independently —H; and
Q is W.
   (J) For example, in one embodiment:
X is independently N;
R^{A2} is independently —H;
R^{A2} is as defined herein;
each of R^{A3}, R^{A5}, R^{B2}, R^{B4}, R^{B5}, and R^{B6}, if present, is independently —H; and
Q is W;
   as in, for example:

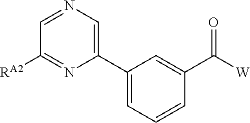

(K) For example, in one embodiment:
X is independently N;
R^{A1} is independently —NH₂;
R^{A2} is as defined herein;
each of R^{A3}, R^{A5}, R^{B2}, R^{B5}, and R^{B6}, if present, is independently —H; and
Q is W;
   as in, for example:

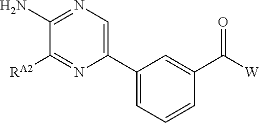

(L) For example, in one embodiment:
X is independently N;
R^{A1} is independently —NH₂;
R^{A2} is independently phenyl, and is independently unsubstituted or substituted;
each of R^{A3}, R^{A5}, R^{B2}, R^{B4}, R^{B5}, and R^{B6}, if present, is independently —H; and
Q is W.
   (M) For example, in one embodiment:
X is independently N;
R^{A1}, is independently —NH₂;
R^{A2} is independently pyridyl, and is independently unsubstituted or substituted;
each of R^{A3}, R^{A5}, R^{B2}, R^{B4}, R^{B5}, and R^{B6}, if present, is independently —H; and
Q is W.
   (N) For example, in one embodiment:
X is independently N;
R^{A1} is independently —NH₂;
R^{A2} is independently pyrimidinyl, and is independently unsubstituted or substituted;
each of R^{A3}, R^{A5}, R^{B2}, R^{B4}, R^{B5}, and R^{B6}, if present, is independently —H; and
Q is W.
   (O) For example, in one embodiment:
X is independently N;
R^{A1} is independently —NH₂;
R^{A2} is independently naphthyl, and is independently unsubstituted or substituted;
each of R^{A3}, R^{A5}, R^{B2}, R^{B4}, R^{B5}, and R^{B6}, if present, is independently —H; and
Q is W.

### Molecular Weight

In one embodiment, the compound has a molecular weight of 270 to 1200.

In one embodiment, the bottom of range is 275; 300; 325; 350; 375; 400.

In one embodiment, the top of range is 1100; 1000, 900, 800, 700.

In one embodiment, the range is 300 to 700.

### Substantially Purified Forms

Another aspect of the present invention pertains to compounds, as described herein, in substantially purified form and/or in a form substantially free from contaminants.

In one embodiment, the substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the substantially purified form is at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

### Some Preferred Embodiments

Examples of some preferred compounds include the following compounds, and pharmaceutically acceptable salts, solvates, amides, esters, ethers, N-oxides, chemically protected forms, and prodrugs thereof.

Examples of compounds where X is N (“pyrazine benzamide compounds”) include the following:

| # | Structure | Name | ID No. |
|---|---|---|---|
| 1 | 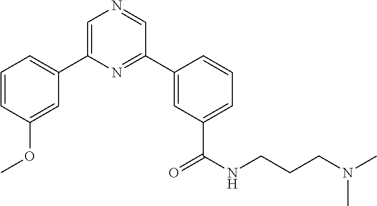 | N-(3- Dimethylamino- propyl)-3-[6-(3- methoxy-phenyl)- pyrazin-2-yl]- benzamide | X-001 (B01) |
| 2 | 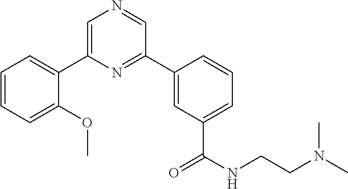 | N-(2- Dimethylamino- ethyl)-3-[6-(2- methoxy-phenyl)- pyrazin-2-yl]- benzamide | X-002 (B02) |
| 3 | 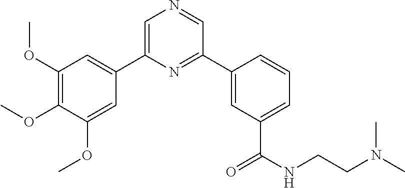 | N-(2- Dimethylamino- ethyl)-3-[6-(3,4,5- trimethoxy- phenyl)-pyrazin-2- yl]-benzamide | X-003 (B03) |
| 4 | 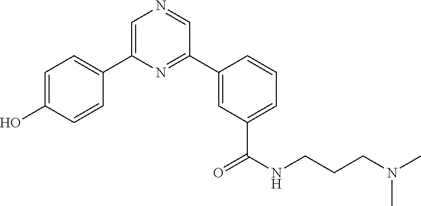 | N-(3- Dimethylamino- propyl)-3-[6-(4- hydroxy-phenyl)- pyrazin-2-yl]- benzamide | X-004 (B04) |
| 5 | 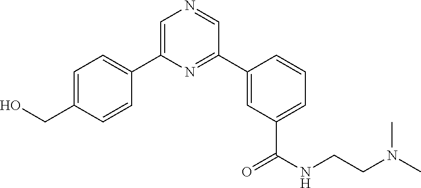 | N-(2- Dimethylamino- ethyl)-3-[6-(4- hydroxymethyl- phenyl)-pyrazin-2- yl]-benzamide | X-005 (B05) |
| 6 | 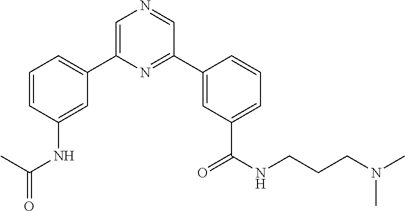 | 3-[6-(3- Acetylamino- phenyl)-pyrazin-2- yl]-N-(3- dimethylamino- propyl)-benzamide | X-006 (B06) |
| 7 | 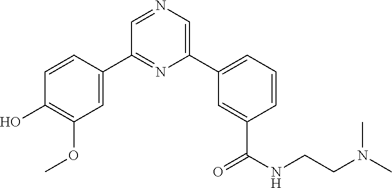 | N-(2- Dimethylamino- ethyl)-3-[6-(4- hydroxy-3- methoxy-phenyl)- pyrazin-2-yl]- benzamide | X-007 (B07) |

Additional examples of compounds where X is N (“pyrazine benzamide compounds”) include the following:

| # | Structure | Name | ID No. |
|---|---|---|---|
| 8 | 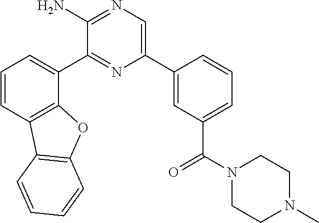 | [3-(5-Amino-6- dibenzofuran-4-yl- pyrazin-2-yl)- phenyl]-(4-methyl- piperazin-1-yl)- methanone | X-008 |
| 9 | 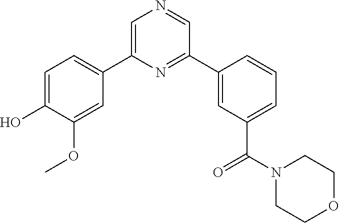 | {3-[6-(4-Hydroxy- 3-methoxy- phenyl)-pyrazin-2- yl]-phenyl}- morpholin-4-yl- methanone | X-009 |
| 10 | 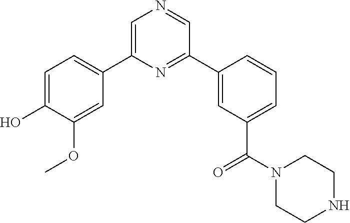 | {3-[6-(4-Hydroxy- 3-methoxy- phenyl)-pyrazin-2- yl]-phenyl}- piperazin-1-yl- methanone | X-010 |
| 11 | 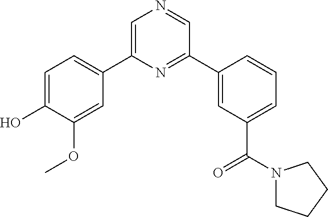 | {3-[6-(4-Hydroxy- 3-methoxy- phenyl)-pyrazin-2- yl]-phenyl}- pyrrolidin-1-yl- methanone | X-011 |
| 12 | 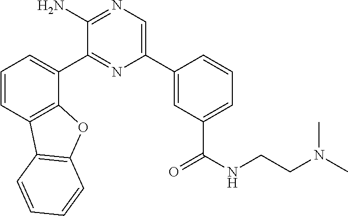 | 3-(5-Amino-6- dibenzofuran-4-yl- pyrazin-2-yl)-N-(2- dimethylamino- ethyl)-benzamide | X-012 |
| 13 | 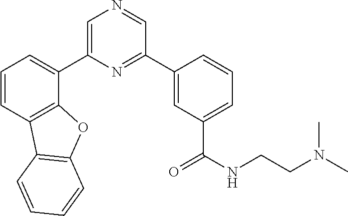 | 3-(6- Dibenzofuran-4-yl- pyrazin-2-yl)-N-(2- dimethylamino- ethyl)-benzamide | X-013 |
| 14 | 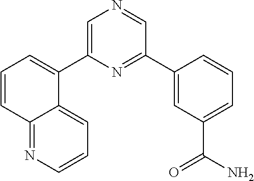 | 3-(6-Quinolin-5-yl- pyrazin-2-yl)- benzamide | X-014 |
| 15 | 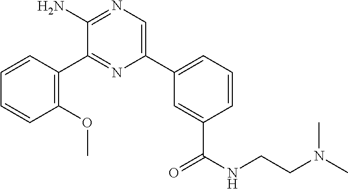 | 3-[5-Amino-6-(2- methoxy-phenyl)- pyrazin-2-yl]-N-(2- dimethylamino- ethyl)-benzamide | X-015 |
| 16 | 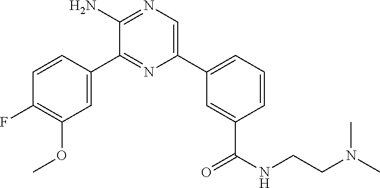 | 3-[5-Amino-6-(4- fluoro-3-methoxy- phenyl)-pyrazin-2- yl]-N-(2- dimethylamino- ethyl)-benzamide | X-016 |
| 17 | 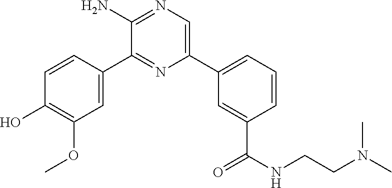 | 3-[5-Amino-6-(4- hydroxy-3- methoxy-phenyl)- pyrazin-2-yl]-N-(2- dimethylamino- ethyl)-benzamide | X-017 |
| 18 | 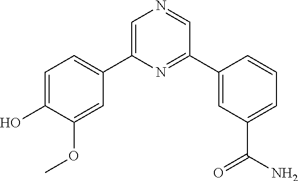 | 3-[6-(4-Hydroxy-3- methoxy-phenyl)- pyrazin-2-yl]- benzamide | X-018 |
| 19 | 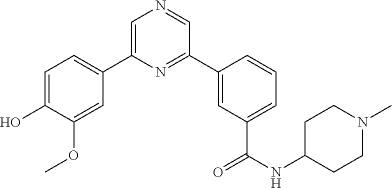 | 3-[6-(4-Hydroxy-3- methoxy-phenyl)- pyrazin-2-yl]-N-(1- methyl-piperidin-4- yl)-benzamide | X-019 |
| 20 | 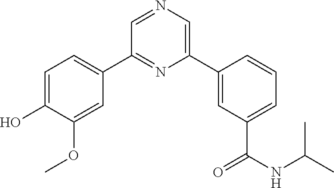 | 3-[6-(4-Hydroxy-3- methoxy-phenyl)- pyrazin-2-yl]-N- isopropyl- benzamide | X-020 |
| 21 | 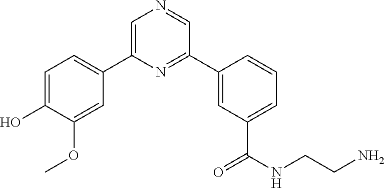 | N-(2-Amino-ethyl)- 3-[6-(4-hydroxy-3- methoxy-phenyl)- pyrazin-2-yl]- benzamide | X-021 |
| 22 | 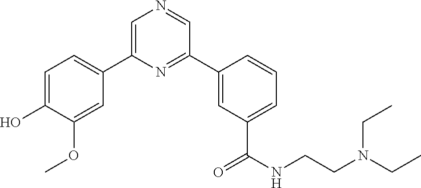 | N-(2- Diethylamino- ethyl)-3-[6-(4- hydroxy-3- methoxy-phenyl)- pyrazin-2-yl]- benzamide | X-022 |
| 23 | 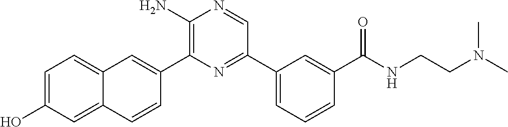 | 3-[5-Amino-6-(6- hydroxy- naphthalen-2-yl)- pyrazin-2-yl]-N-(2- dimethylamino- ethyl)-benzamide | X-023 |
| 24 | 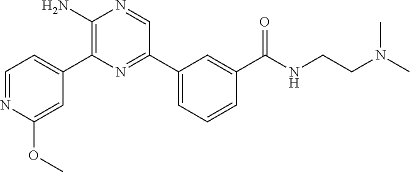 | 3-[5-Amino-6-(2- methoxy-pyridin-4- yl)-pyrazin-2-yl]-N- (2-dimethylamino- ethyl)-benzamide | X-024 |
| 25 | 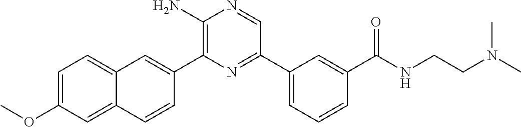 | 3-[5-Amino-6-(6- methoxy- naphthalen-2-yl)- pyrazin-2-yl]-N-(2- dimethylamino- ethyl)-benzamide | X-025 |
| 26 | 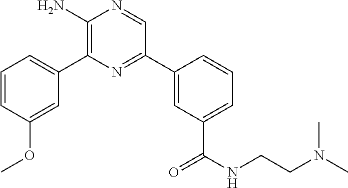 | 3-[5-Amino-6-(3- methoxy-phenyl)- pyrazin-2-yl]-N-(2- dimethylamino- ethyl)-benzamide | X-026 |
| 27 | 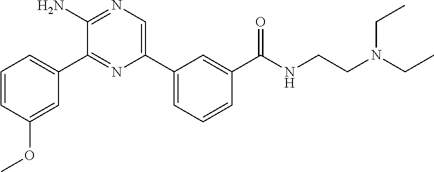 | 3-[5-Amino-6-(3- methoxy-phenyl)- pyrazin-2-yl]-N-(2- diethylamino- ethyl)-benzamide | X-027 |

Examples of compounds where X is C(R^{A3}) (“pyridine benzamide compounds”) include the following:

| # | Structure | Name | ID No. |
|---|---|---|---|
| 28 | 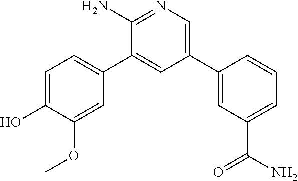 | 3-[6-Amino-5-(4- hydroxy-3- methoxy-phenyl)- pyridin-3-yl]- benzamide | Y-001 (B08) |
| 29 | 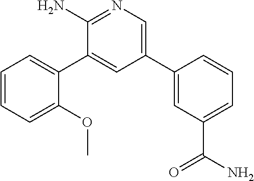 | 3-[6-Amino-5-(2- methoxy-phenyl)- pyridin-3-yl] benzamide | Y-002 (B09) |
| 30 | 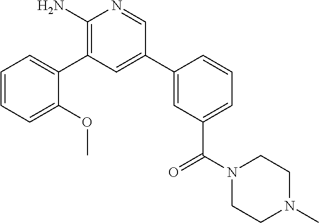 | {3-[6-Amino-5-(2- methoxy-phenyl)- pyridin-3-yl]- phenyl}-(4-methyl- piperazin-1-yl)- methanone | Y-003 (B10) |

Additional examples of compounds where X is C(R^{A3}) (“pyridine benzamide compounds”) include the following:

| # | Structure | Name | ID No. |
|---|---|---|---|
| 31 | 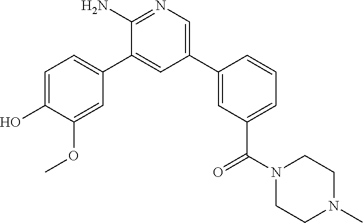 | {3-[6-Amino-5-(4- hydroxy-3- methoxy-phenyl)- pyridin-3-yl]- phenyl}-(4-methyl- piperazin-1-yl)- methanone | Y-004 |
| 32 | 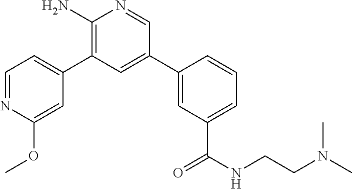 | 3-(2-Amino-2′- methoxy- [3,4′]bipyridinyl-5- yl)-N-(2- dimethylamino- ethyl)-benzamide | Y-005 |
| 33 | 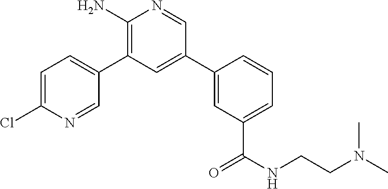 | 3-(2-Amino-6′- chloro- [3,3′]bipyridinyl-5- yl)-N-(2- dimethylamino- ethyl)-benzamide | Y-006 |
| 34 | 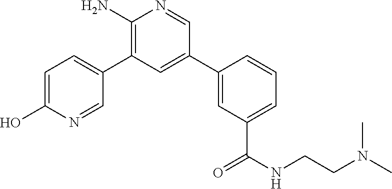 | 3-(2-Amino-6′- hydroxy- [3,3′]bipyridinyl-5- yl)-N-(2- dimethylamino- ethyl)-benzamide | Y-007 |
| 35 | 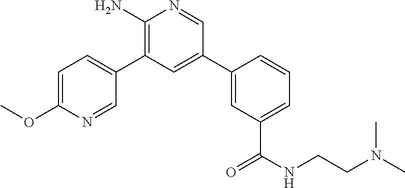 | 3-(2-Amino-6′- methoxy- [3,3′]bipyridinyl-5- yl)-N-(2- dimethylamino- ethyl)-benzamide | Y-008 |
| 36 | 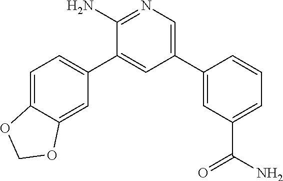 | 3-(6-Amino-5- benzo[1,3]dioxol- 5-yl-pyridin-3-yl)- benzamide | Y-009 |
| 37 | 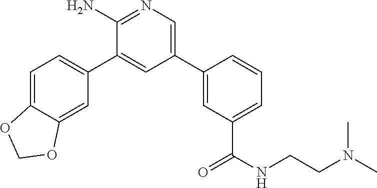 | 3-(6-Amino-5- benzo[1,3]dioxol- 5-yl-pyridin-3-yl)- N-(2- dimethylamino- ethyl)-benzamide | Y-010 |
| 38 | 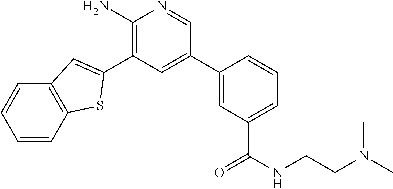 | 3-(6-Amino-5- benzo[b]thiophen- 2-yl-pyridin-3-yl)- N-(2- dimethylamino- ethyl)-benzamide | Y-011 |
| 39 | 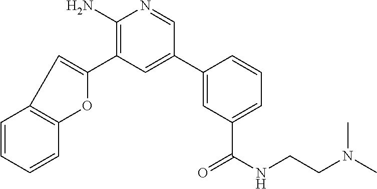 | 3-(6-Amino-5- benzofuran-2-yl- pyridin-3-yl)-N-(2- dimethylamino- ethyl)-benzamide | Y-012 |
| 40 | 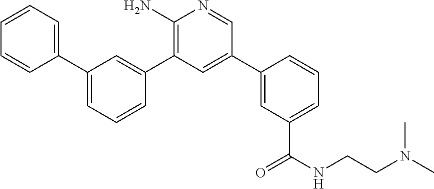 | 3-(6-Amino-5- biphenyl-3-yl- pyridin-3-yl)-N-(2- dimethylamino- ethyl)-benzamide | Y-013 |
| 41 | 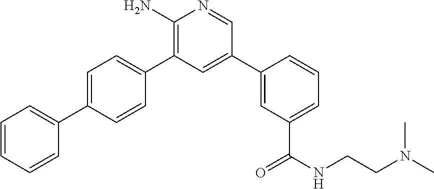 | 3-(6-Amino-5- biphenyl-4-yl- pyridin-3-yl)-N-(2- dimethylamino- ethyl)-benzamide | Y-014 |
| 42 | 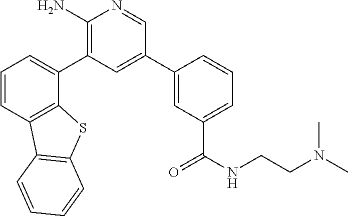 | 3-(6-Amino-5- dibenzothiophen- 4-yl-pyridin-3-yl)- N-(2- dimethylamino- ethyl)-benzamide | Y-015 |
| 43 | 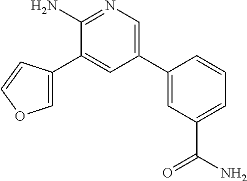 | 3-(6-Amino-5- furan-3-yl-pyridin- 3-yl)-benzamide | Y-016 |
| 44 | 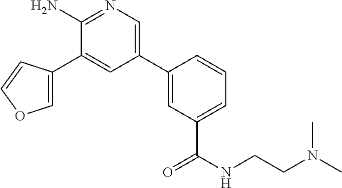 | 3-(6-Amino-5- furan-3-yl-pyridin- 3-yl)-N-(2- dimethylamino- ethyl)-benzamide | Y-017 |
| 45 | 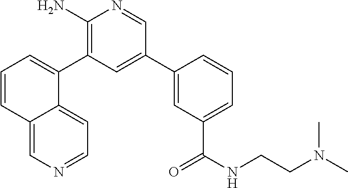 | 3-(6-Amino-5- isoquinolin-5-yl- pyridin-3-yl)-N-(2- dimethylamino- ethyl)-benzamide | Y-018 |
| 46 | 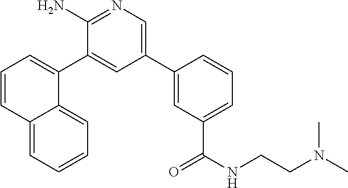 | 3-(6-Amino-5- naphthalen-1-yl- pyridin-3-yl)-N-(2- dimethylamino- ethyl)-benzamide | Y-019 |
| 47 | 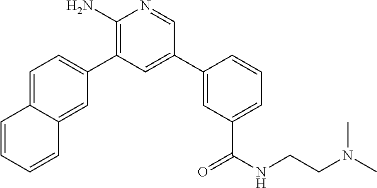 | 3-(6-Amino-5- naphthalen-2-yl- pyridin-3-yl)-N-(2- dimethylamino- ethyl)-benzamide | Y-020 |
| 48 | 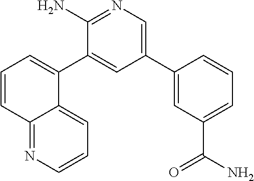 | 3-(6-Amino-5- quinolin-5-yl- pyridin-3-yl)- benzamide | Y-021 |
| 49 | 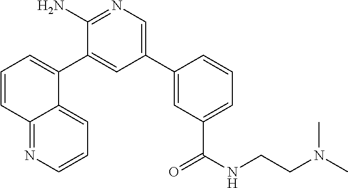 | 3-(6-Amino-5- quinolin-5-yl- pyridin-3-yl)-N-(2- dimethylamino- ethyl)-benzamide | Y-022 |
| 50 | 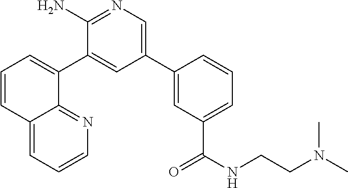 | 3-(6-Amino-5- quinolin-8-yl- pyridin-3-yl)-N-(2- dimethylamino- ethyl)-benzamide | Y-023 |
| 51 | 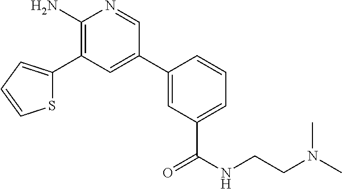 | 3-(6-Amino-5- thiophen-2-yl- pyridin-3-yl)-N-(2- dimethylamino- ethyl)-benzamide | Y-024 |
| 52 | 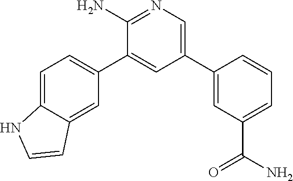 | 3-[6-Amino-5-(1H- indol-5-yl)-pyridin- 3-yl]-benzamide | Y-025 |
| 53 | 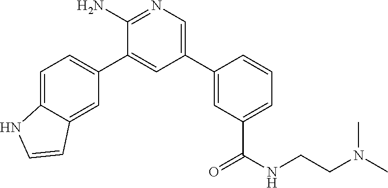 | 3-[6-Amino-5-(1H- indol-5-yl)-pyridin- 3-yl]-N-(2- dimethylamino- ethyl)-benzamide | Y-026 |
| 54 | 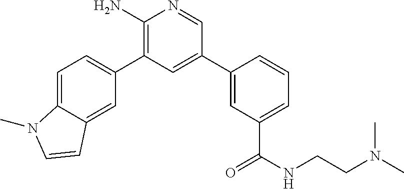 | 3-[6-Amino-5-(1- methyl-1H-indol-5- yl)-pyridin-3-yl]-N- (2-dimethylamino- ethyl)-benzamide | Y-027 |
| 55 | 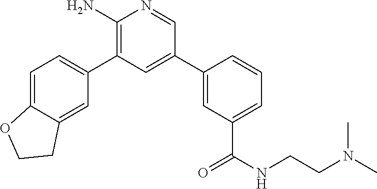 | 3-[6-Amino-5-(2,3- dihydro- benzofuran-5-yl)- pyridin-3-yl]-N-(2- dimethylamino- ethyl)-benzamide | Y-028 |
| 56 | 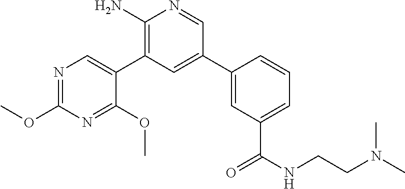 | 3-[6-Amino-5-(2,4- dimethoxy- pyrimidin-5-yl)- pyridin-3-yl]-N-(2- dimethylamino- ethyl)-benzamide | Y-029 |
| 57 | 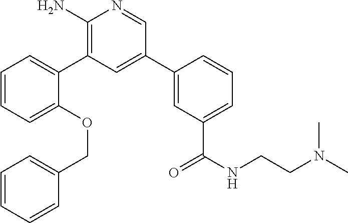 | 3-[6-Amino-5-(2- benzyloxy- phenyl)-pyridin-3- yl]-N-(2- dimethylamino- ethyl)-benzamide | Y-030 |
| 58 | 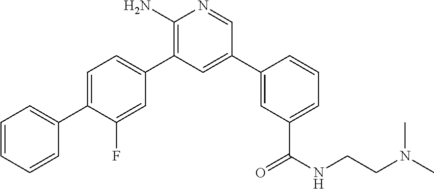 | 3-[6-Amino-5-(2- fluoro-biphenyl-4- yl)-pyridin-3-yl]-N- (2-dimethylamino- ethyl)-benzamide | Y-031 |
| 59 | 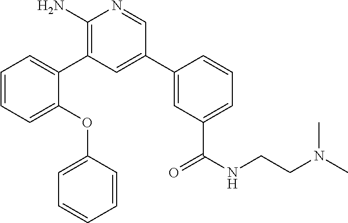 | 3-[6-Amino-5-(2- phenoxy-phenyl)- pyridin-3-yl]-N-(2- dimethylamino- ethyl)-benzamide | Y-032 |
| 60 | 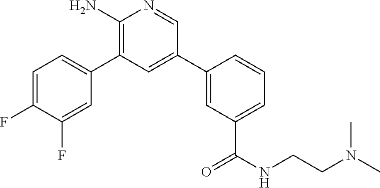 | 3-[6-Amino-5-(3,4- difluoro-phenyl)- pyridin-3-yl]-N-(2- dimethylamino- ethyl)-benzamide | Y-033 |
| 61 | 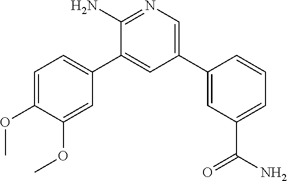 | 3-[6-Amino-5-(3,4- dimethoxy- phenyl)-pyridin-3- yl]-benzamide | Y-034 |
| 62 | 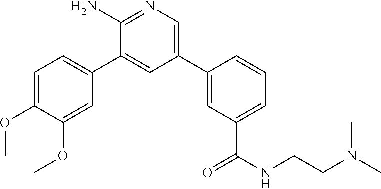 | 3-[6-Amino-5-(3,4- dimethoxy- phenyl)-pyridin-3- yl]-N-(2- dimethylamino- ethyl)-benzamide | Y-035 |
| 63 | 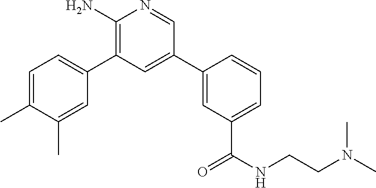 | 3-[6-Amino-5-(3,4- dimethyl-phenyl)- pyridin-3-yl]-N-(2- dimethylamino- ethyl)-benzamide | Y-036 |
| 64 | 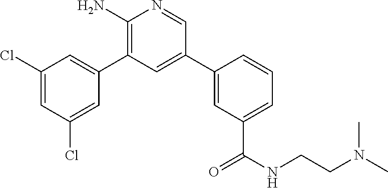 | 3-[6-Amino-5-(3,5- dichloro-phenyl)- pyridin-3-yl]-N-(2- dimethylamino- ethyl)-benzamide | Y-037 |
| 65 | 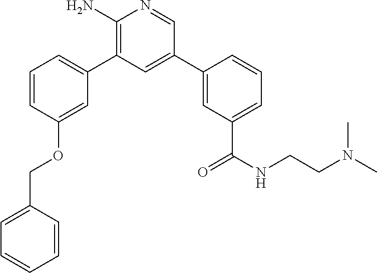 | 3-[6-Amino-5-(3- benzyloxy- phenyl)-pyridin-3- yl]-N-(2- dimethylamino- ethyl)-benzamide | Y-038 |
| 66 | 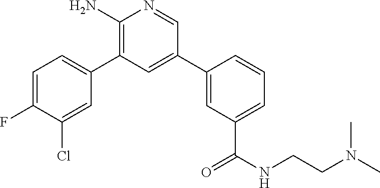 | 3-[6-Amino-5-(3- chloro-4-fluoro- phenyl)-pyridin-3- yl]-N-(2- dimethylamino- ethyl)-benzamide | Y-039 |
| 67 | 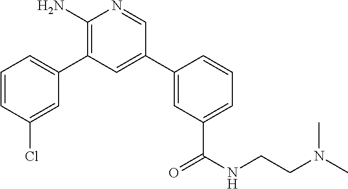 | 3-[6-Amino-5-(3- chloro-phenyl)- pyridin-3-yl]-N-(2- dimethylamino- ethyl)-benzamide | Y-040 |
| 68 | 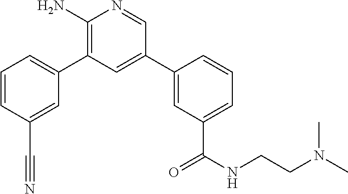 | 3-[6-Amino-5-(3- cyano-phenyl)- pyridin-3-yl]-N-(2- dimethylamino- ethyl)-benzamide | Y-041 |
| 69 | 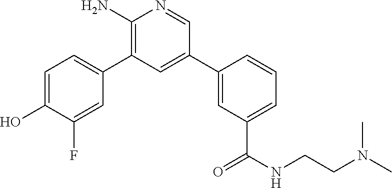 | 3-[6-Amino-5-(3- fluoro-4-hydroxy- phenyl)-pyridin-3- yl]-N-(2- dimethylamino- ethyl)-benzamide | Y-042 |
| 70 | 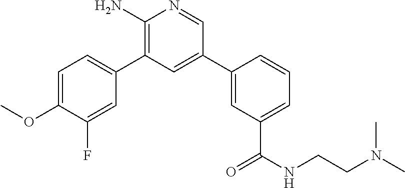 | 3-[6-Amino-5-(3- fluoro-4-methoxy- phenyl)-pyridin-3- yl]-N-(2- dimethylamino- ethyl)-benzamide | Y-043 |
| 71 | 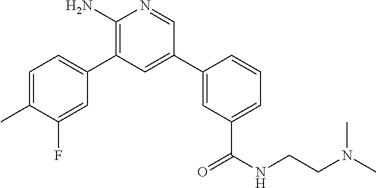 | 3-[6-Amino-5-(3- fluoro-4-methyl- phenyl)-pyridin-3- yl]-N-(2- dimethylamino- ethyl)-benzamide | Y-044 |
| 72 | 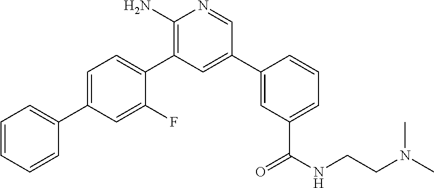 | 3-[6-Amino-5-(3- fluoro-biphenyl-4- yl)-pyridin-3-yl]-N- (2-dimethylamino- ethyl)-benzamide | Y-045 |
| 73 | 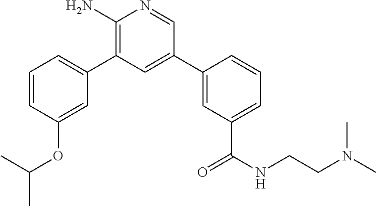 | 3-[6-Amino-5-(3- isopropoxy- phenyl)-pyridin-3- yl]-N-(2- dimethylamino- ethyl)-benzamide | Y-046 |
| 74 | 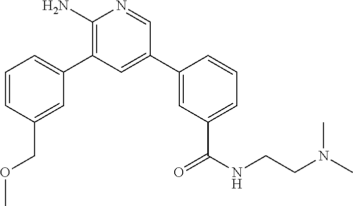 | 3-[6-Amino-5-(3- methoxymethyl- phenyl)-pyridin-3- yl]-N-(2- dimethylamino- ethyl)-benzamide | Y-047 |
| 75 | 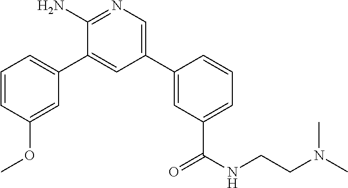 | 3-[6-Amino-5-(3- methoxy-phenyl)- pyridin-3-yl]-N-(2- dimethylamino- ethyl)-benzamide | Y-048 |
| 76 | 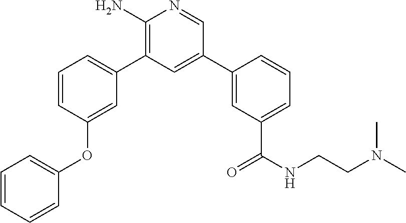 | 3-[6-Amino-5-(3- phenoxy-phenyl)- pyridin-3-yl]-N-(2- dimethylamino- ethyl)-benzamide | Y-049 |
| 77 | 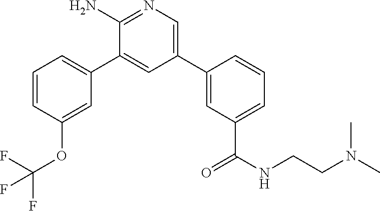 | 3-[6-Amino-5-(3- trifluoromethoxy- phenyl)-pyridin-3- yl]-N-(2- dimethylamino- ethyl)-benzamide | Y-050 |
| 78 | 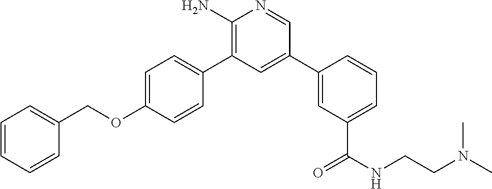 | 3-[6-Amino-5-(4- benzyloxy- phenyl)-pyridin-3- yl]-N-(2- dimethylamino- ethyl)-benzamide | Y-051 |
| 79 | 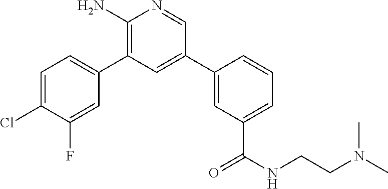 | 3-[6-Amino-5-(4- chloro-3-fluoro- phenyl)-pyridin-3- yl]-N-(2- dimethylamino- ethyl)-benzamide | Y-052 |
| 80 | 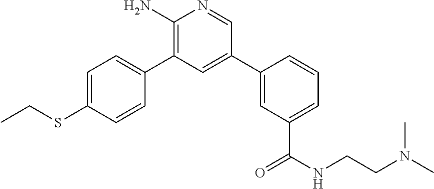 | 3-[6-Amino-5-(4- ethylsulfanyl- phenyl)-pyridin-3- yl]-N-(2- dimethylamino- ethyl)-benzamide | Y-053 |
| 81 | 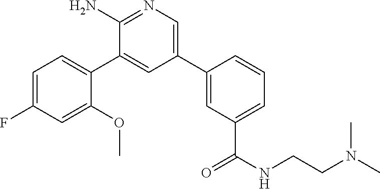 | 3-[6-Amino-5-(4- fluoro-2-methoxy- phenyl)-pyridin-3- yl]-N-(2- dimethylamino- ethyl)-benzamide | Y-054 |
| 82 | 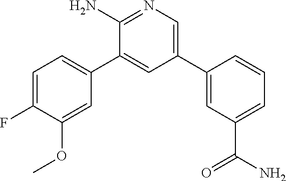 | 3-[6-Amino-5-(4- fluoro-3-methoxy- phenyl)-pyridin-3- yl]-benzamide | Y-055 |
| 83 | 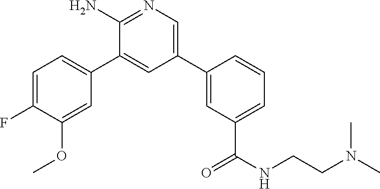 | 3-[6-Amino-5-(4- fluoro-3-methoxy- phenyl)-pyridin-3- yl]-N-(2- dimethylamino- ethyl)-benzamide | Y-056 |
| 84 | 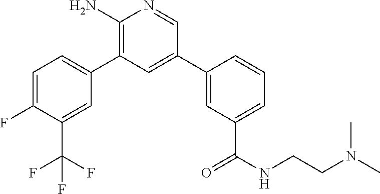 | 3-[6-Amino-5-(4- fluoro-3- trifluoromethyl- phenyl)-pyridin-3- yl]-N-(2- dimethylamino- ethyl)-benzamide | Y-057 |
| 85 | 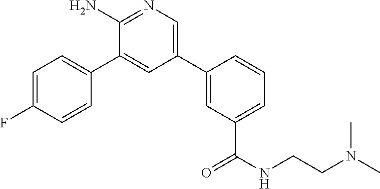 | 3-[6-Amino-5-(4- fluoro-phenyl)- pyridin-3-yl]-N-(2- dimethylamino- ethyl)-benzamide | Y-058 |
| 86 | 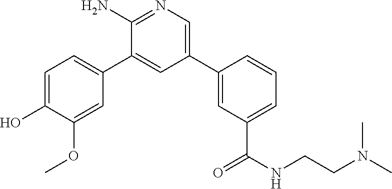 | 3-[6-Amino-5-(4- hydroxy-3- methoxy-phenyl)- pyridin-3-yl]-N-(2- dimethylamino- ethyl)-benzamide | Y-059 |
| 87 | 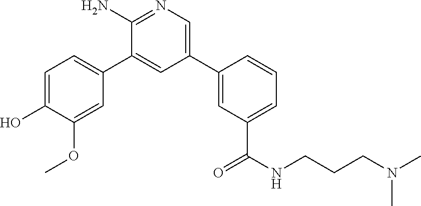 | 3-[6-Amino-5-(4- hydroxy-3- methoxy-phenyl)- pyridin-3-yl]-N-(3- dimethylamino- propyl)-benzamide | Y-060 |
| 88 | 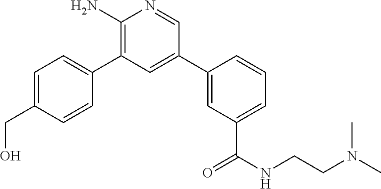 | 3-[6-Amino-5-(4- hydroxymethyl- phenyl)-pyridin-3- yl]-N-(2- dimethylamino- ethyl)-benzamide | Y-061 |
| 89 | 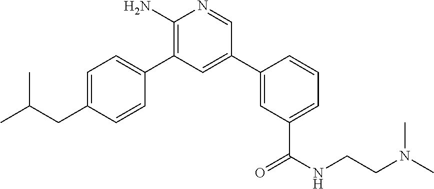 | 3-[6-Amino-5-(4- isobutyl-phenyl)- pyridin-3-yl]-N-(2- dimethylamino- ethyl)-benzamide | Y-062 |
| 90 | 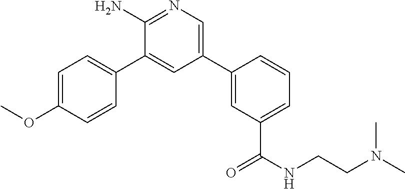 | 3-[6-Amino-5-(4- methoxy-phenyl)- pyridin-3-yl]-N-(2- dimethylamino- ethyl)-benzamide | Y-063 |
| 91 | 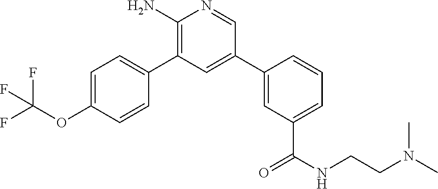 | 3-[6-Amino-5-(4- trifluoromethoxy- phenyl)-pyridin-3- yl]-N-(2- dimethylamino- ethyl)-benzamide | Y-064 |
| 92 | 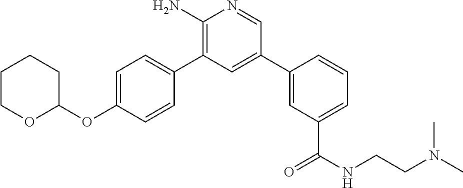 | 3-{6-Amino-5-[4- (tetrahydro-pyran- 2-yloxy)-phenyl]- pyridin-3-yl}-N-(2- dimethylamino- ethyl)-benzamide | Y-065 |
| 93 | 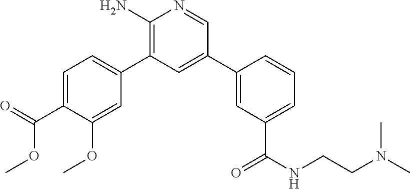 | 4-{2-Amino-5-[3- (2-dimethylamino- ethylcarbamoyl)- phenyl]-pyridin-3- yl}-2-methoxy- benzoic acid methyl ester | Y-066 |
| 94 | 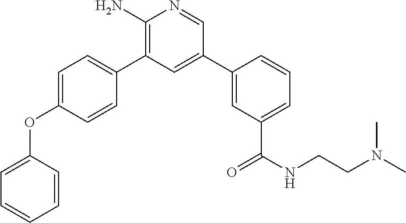 | 3-[6-Amino-5-(4- phenoxy-phenyl)- pyridin-3-yl]-N-(2- dimethylamino- ethyl)-benzamide | Y-067 |
| 95 | 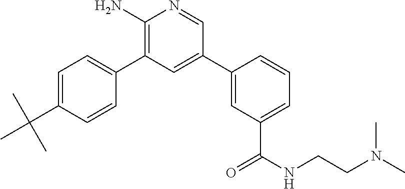 | 3-[6-Amino-5-(4- tert-butyl-phenyl)- pyridin-3-yl]-N-(2- dimethylamino- ethyl)-benzamide | Y-068 |
| 96 | 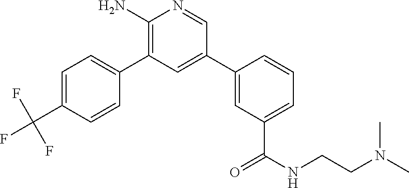 | 3-[6-Amino-5-(4- trifluoromethyl- phenyl)-pyridin-3- yl]-N-(2- dimethylamino- ethyl)-benzamide | Y-069 |
| 97 | 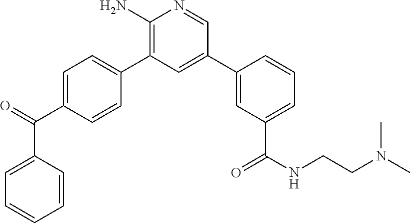 | 3-[6-Amino-5-(4- benzoyl-phenyl)- pyridin-3-yl]-N-(2- dimethylamino- ethyl)-benzamide | Y-070 |
| 98 | 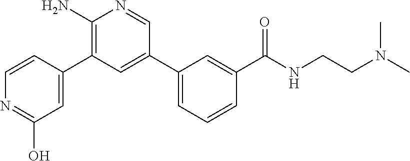 | 3-(2-Amino-2′- hydroxy- [3,4′]bipyridinyl-5- yl)-N-(2- dimethylamino- ethyl)-benzamide | Y-071 |
| 99 | 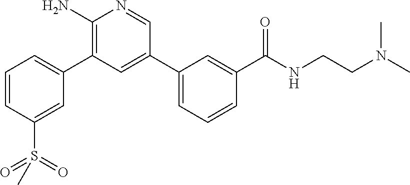 | 3-[6-Amino-5-(3- methanesulfonyl- phenyl)-pyridin-3- yl]-N-(2- dimethylamino- ethyl)-benzamide | Y-072 |
| 100 | 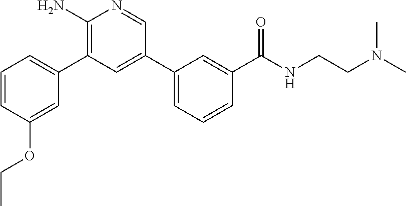 | 3-[6-Amino-5-(3- ethoxy-phenyl)- pyridin-3-yl]-N-(2- dimethylamino- ethyl)-benzamide | Y-073 |
| 101 | 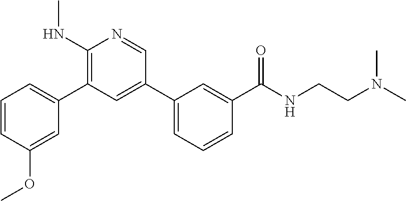 | N-(2- Dimethylamino- ethyl)-3-[5-(3- methoxy-phenyl)- 6-methylamino- pyridin-3-yl]- benzamide | Y-074 |
| 102 | 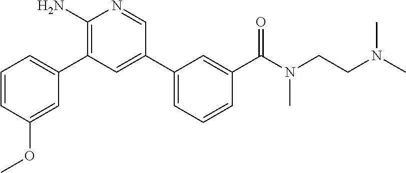 | 3-[6-Amino-5-(3- methoxy-phenyl)- pyridin-3-yl]-(2- dimethylamino- ethyl)-N-methyl- benzamide | Y-075 |
| 103 | 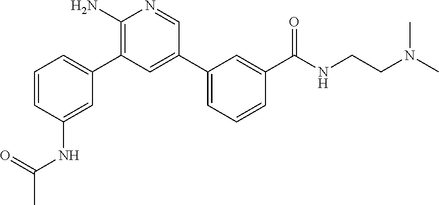 | 3-[5-(3- Acetylamino- phenyl)-6-amino- pyridin-3-yl]-N-(2- dimethylamino- ethyl)-benzamide | Y-076 |
| 104 | 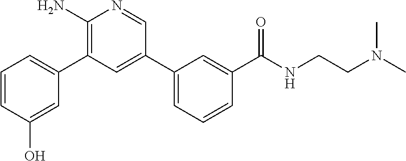 | 3-[6-Amino-5-(3- hydroxy-phenyl)- pyridin-3-yl]-N-(2- dimethylamino- ethyl)-benzamide | Y-077 |
| 105 | 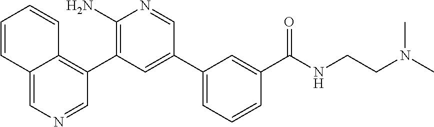 | 3-(6-Amino-5- isoquinolin-4-yl- pyridin-3-yl)-N-(2- dimethylamino- ethyl)-benzamide | Y-078 |
| 106 | 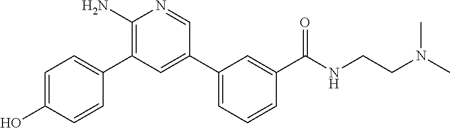 | 3-[6-Amino-5-(4- hydroxy-phenyl)- pyridin-3-yl]-N-(2- dimethylamino- ethyl)-benzamide | Y-079 |
| 107 | 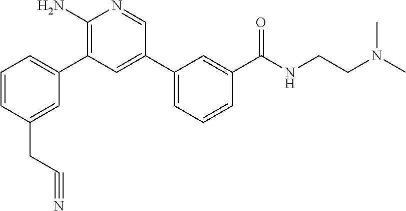 | 3-[6-Amino-5-(3- cyanomethyl- phenyl)-pyridin-3- yl]-N-(2- dimethylamino- ethyl)-benzamide | Y-080 |
| 108 | 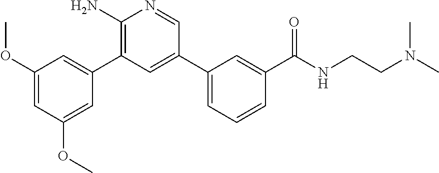 | 3-[6-Amino-5-(3,5- dimethoxy- phenyl)-pyridin-3- yl]-N-(2- dimethylamino- ethyl)-benzamide | Y-081 |
| 109 | 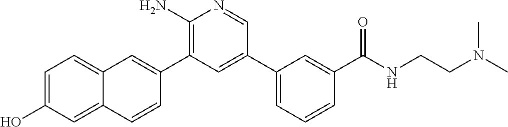 | 3-[6-Amino-5-(6- hydroxy- naphthalen-2-yl)- pyridin-3-yl]-N-(2- dimethylamino- ethyl)-benzamide | Y-082 |
| 110 | 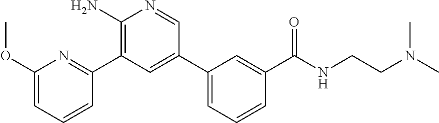 | 3-(2′-Amino-6- methoxy- [2,3′]bipyridinyl-5′- yl)-N-(2- dimethylamino- ethyl)-benzamide | Y-083 |
| 111 | 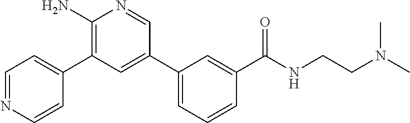 | 3-(2-Amino- [3,4′]bipyridinyl-5- yl)-N-(2- dimethylamino- ethyl)-benzamide | Y-084 |
| 112 | 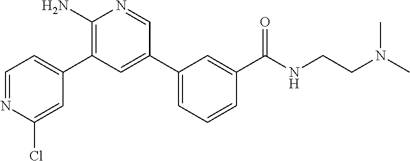 | 3-(2-Amino-2′- chloro- [3,4′]bipyridinyl-5- yl)-N-(2- dimethylamino- ethyl)-benzamide | Y-085 |
| 113 | 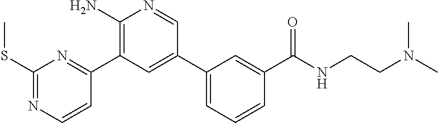 | 3-[6-Amino-5-(2- methylsulfanyl- pyrimidin-4-yl)- pyridin-3-yl]-N-(2- dimethylamino- ethyl)-benzamide | Y-086 |
| 114 | 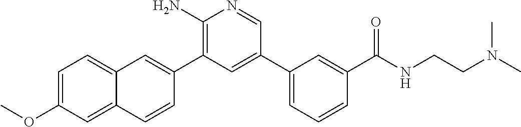 | 3-[6-Amino-5-(6- methoxy- naphthalen-2-yl)- pyridin-3-yl]-N-(2- dimethylamino- ethyl)-benzamide | Y-087 |
| 115 | 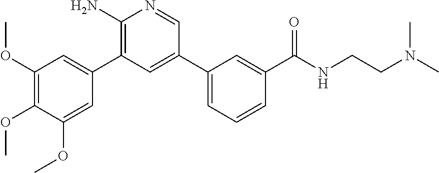 | 3-[6-Amino-5- (3,4,5-trimethoxy- phenyl)-pyridin-3- yl]-N-(2- dimethylamino- ethyl)-benzamide | Y-088 |
| 116 | 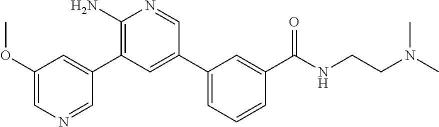 | 3-(2-Amino-5′- methoxy- [3,3′]bipyridinyl-5- yl)-N-(2- dimethylamino- ethyl)-benzamide | Y-089 |
| 117 | 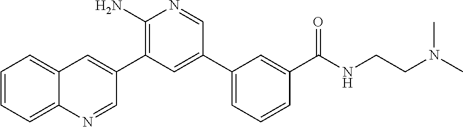 | 3-(6-Amino-5- quinolin-3-yl- pyridin-3-yl)-N-(2- dimethylamino- ethyl)-benzamide | Y-090 |
| 118 | 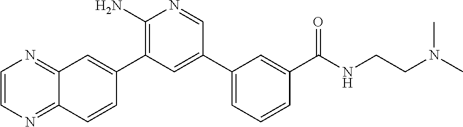 | 3-(6-Amino-5- quinoxalin-6-yl- pyridin-3-yl)-N-(2- dimethylamino- ethyl)-benzamide | Y-091 |
| 119 | 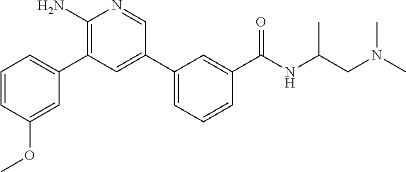 | 3-[6-Amino-5-(3- methoxy-phenyl)- pyridin-3-yl]-N-(2- dimethylamino-1- methyl-ethyl)- benzamide | Y-092 |
| 120 | 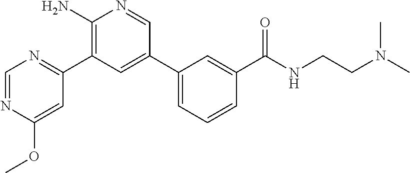 | 3-[6-Amino-5-(6- methoxy- pyrimidin-4-yl)- pyridin-3-yl]-N-(2- dimethylamino- ethyl)-benzamide | Y-093 |
| 121 | 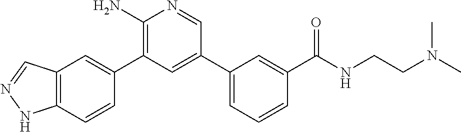 | 3-[6-Amino-5-(1H- indazol-5-yl)- pyridin-3-yl]-N-(2- dimethylamino- ethyl)-benzamide | Y-094 |
| 122 | 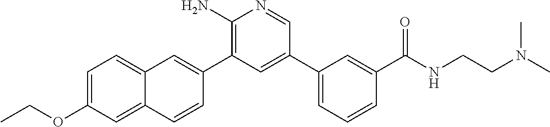 | 3-[6-Amino-5-(6- ethoxy- naphthalen-2-yl)- pyridin-3-yl]-N-(2- dimethylamino- ethyl)-benzamide | Y-095 |
| 123 | 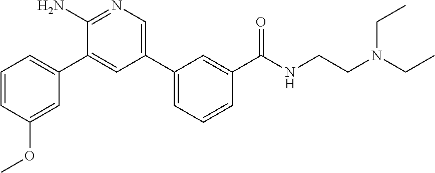 | 3-[6-Amino-5-(3- methoxy-phenyl)- pyridin-3-yl]-N-(2- diethylamino- ethyl)-benzamide | Y-096 |

### DEFINITIONS

The term “alkyl,” as used herein, pertains to a monovalent moiety obtained by removing a hydrogen atom from a carbon atom of a saturated aliphatic hydrocarbon compound having from 1 to 20 carbon atoms (unless otherwise specified).

In one embodiment, each C₁₋₇alkyl is independently selected from: -Me, -Et, -nPr, -iPr, -nBu, -iBu, -sBu, -tBu, n-pentyl, i-pentyl, neo-pentyl, n-hexyl, n-heptyl; and is independently unsubstituted or substituted. In one embodiment, each C₁₋₇alkyl is independently unsubstituted.

The term “alkylenyl,” as used herein, pertains to a divalent bidentate moiety obtained by removing two hydrogen atoms from one carbon atom or two different carbon atoms of a saturated aliphatic hydrocarbon compound having from 1 to 20 carbon atoms (unless otherwise specified).

In one embodiment, each C₁₋₇alkylenyl is independently selected from: —CH₂—, —CH₂CH₂—, —CH₂CH₂CH₂—, —CH₂CH₂CH₂CH₂—, —CH(CH₃)—, —CH(CH₂CH₃)—, —CH(CH₃)CH₂—, —CH₂CH(CH₃)—, —CH(CH₃)CH₂CH₂—, and —CH₂CH₂CH(CH₃)—, and is independently unsubstituted or substituted. In one embodiment, each C₁₋₇alkylenyl is independently unsubstituted.

The term “alkenyl,” as used herein, pertains to a monovalent moiety obtained by removing a hydrogen atom from a carbon atom of an unsaturated aliphatic hydrocarbon compound having from 1 to 20 carbon atoms (unless otherwise specified) and having one or more (e.g., 1, 2, etc.) carbon-carbon double bonds.

In one embodiment, each C₂₋₇alkenyl is independently selected from: —CH═CH₂, —CH═CH—CH₃, —CH—CH═CH₂, —C(CH₃)═CH₂, and butenyl (C₄₋); and is independently unsubstituted or substituted. In one embodiment, each C₂₋₇alkenyl is independently unsubstituted.

The term “alkynyl,” as used herein, pertains to a monovalent moiety obtained by removing a hydrogen atom from a carbon atom of an unsaturated aliphatic hydrocarbon compound having from 1 to 20 carbon atoms (unless otherwise specified) and having one or more (e.g., 1, 2, etc.) carbon-carbon triple bonds.

In one embodiment, each C₂₋₇alkynyl is independently selected from: —C≡CH and —CH₂—C≡CH; and is independently unsubstituted or substituted. In one embodiment, each C₂₋₇alkynyl is independently unsubstituted.

The term “cycloalkyl,” as used herein, pertains to a monovalent moiety obtained by removing a hydrogen atom from a ring carbon atom of a saturated hydrocarbon compound having at least one carbocyclic ring, and having from 3 to 20 carbon atoms (unless otherwise specified), including from 3 to 20 ring atoms (unless otherwise specified).

In one embodiment, each C₃₋₇cycloalkyl is independently selected from: cyclopropyl (C₃), cyclobutyl (C₄), cyclopentyl (C₅), cyclohexyl (C₆), cycloheptyl (C₇), methylcyclopropyl (C₄), dimethylcyclopropyl (C₅), methylcyclobutyl (C₅), dimethylcyclobutyl (C₆), methylcyclopentyl (C₆), dimethylcyclopentyl (C₇), methylcyclohexyl (C₇); and is independently unsubstituted or substituted. In one embodiment, each C₃₋₇cycloalkyl is independently unsubstituted.

The term “cycloalkenyl,” as used herein, pertains to a monovalent moiety obtained by removing a hydrogen atom from a ring carbon atom of an unsaturated hydrocarbon compound having at least one carbocyclic ring that has at least one carbon-carbon double bond as part of that ring, and having from 3 to 20 carbon atoms (unless otherwise specified), including from 3 to 20 ring atoms (unless otherwise specified).

In one embodiment, each C₃₋₇cycloalkenyl is independently selected from: cyclopropenyl (C₃), cyclobutenyl (C₄), cyclopentenyl (C₅), cyclohexenyl (C₆), methylcyclopropenyl (C₄), dimethylcyclopropenyl (C₅), methylcyclobutenyl (C₅), dimethylcyclobutenyl (C₆), methylcyclopentenyl (C₆), dimethylcyclopentenyl (C₇), methylcyclohexenyl (C₇); and is independently unsubstituted or substituted. In one embodiment, each C₃₋₇cycloalkenyl is independently unsubstituted.

The term “heterocyclyl,” as used herein, pertains to a monovalent moiety obtained by removing a hydrogen atom from a non-aromatic ring atom of a compound having at least one non-aromatic heterocyclic ring, and having from 3 to 20 carbon atoms (unless otherwise specified), including from 3 to 20 ring atoms (unless otherwise specified), of which from 1 to 10 are ring heteroatoms (unless otherwise specified). Preferably, each ring of the compound has from 3 to 7 ring atoms, of which from 1 to 4 are ring heteroatoms. Preferably, the ring heteroatoms are selected from N, O, and S.

In this context, the prefixes (e.g., C₃₋₁₄, C₃₋₇, C₅₋₆, etc.) denote the number of ring atoms, or range of number of ring atoms, whether carbon atoms or heteroatoms. For example, the term “C₅₋₆heterocyclyl,” as used herein, pertains to a heterocyclyl group having 5 or 6 ring atoms.

In one embodiment, each O₃₋₁₄heterocyclyl is independently selected from: C₃heterocyclyl groups including:
Nᵢ: aziridinyl (C₃);
O₁: oxiranyl (C₃); and
S₁: thiiranyl (C₃);

C₄heterocyclyl groups including:
N₁: azetidinyl (C₄);
O₁: oxetanyl (C₄); and
S₁: thietanyl (C₄);

C₅heterocyclyl groups including:
N₁: pyrrolidinyl (C₅), pyrrolinyl (C₅), 2H-pyrrolyl or 3H-pyrrolyl (C₅);
O₁: tetrahydrofuranyl (C₅), dihydrofuranyl (C₅);
S₁: tetrahydrothienyl (C₅);
O₂: dioxolanyl (C₅);
N₂: imidazolidinyl (C₅), pyrazolidinyl (C₅), imidazolinyl (C₅), pyrazolinyl (C₅);
N₁O₁: tetrahydrooxazolyl (C₅), dihydrooxazolyl (C₅), tetrahydroisoxazolyl (C₅), dihydroisoxazolyl (C₅);
N₁S₁: thiazolinyl (C₅), thiazolidinyl (C₅); and
O₁S₁: oxathiolyl (C₅);

C₆heterocyclyl groups including:
N₁: piperidinyl (C₆), dihydropyridinyl (C₆), tetrahydropyridinyl (C₆);
O₁: tetrahydropyranyl (C₆), dihydropyranyl (C₆), pyranyl (C₆);
S₁: tetrahydrothiopyranyl (C₆);
O₂: dioxanyl (C₆);
O₃: trioxanyl (C₆);
N₂: piperazinyl (C₆);
N₁O₁: morpholinyl (C₆), tetrahydrooxazinyl (C₆), dihydrooxazinyl (C₆), oxazinyl (C₆);
N₁S₁: thiomorpholinyl (C₆);
N₂O₁: oxadiazinyl (C₆);
O₁S₁: oxathianyl (thioxanyl) (C₆); and,
N₁O₁S₁: oxathiazinyl (C₆); and

C₇heterocyclyl groups including:
N₁: azepinyl (C₇);
O₁: oxepinyl (C₇);
S₁: thiepanyl (C₇); and
O₂: dioxepanyl (C₇).

The term “aryl,” as used herein, pertains to a monovalent moiety obtained by removing a hydrogen atom from an aromatic ring atom of an aromatic compound, which moiety has from 3 to 20 ring atoms (unless otherwise specified). Preferably, each ring has from 5 to 7 ring atoms, of which from 0 to 4 are ring heteroatoms.

The ring atoms may be all carbon atoms, as in “carboaryl” groups. Alternatively, the ring atoms may include one or more heteroatoms, as in “heteroaryl” groups. Preferably, the ring heteroatoms are selected from N, O, and S.

In this context, the prefixes (e.g., C₃₋₁₄, C₅₋₇, C₆₋₆, etc.) denote the number of ring atoms, or range of number of ring atoms, whether carbon atoms or heteroatoms. For example, the term “C₅₋₆heteroaryl,” as used herein, pertains to a heteroaryl group having 5 or 6 ring atoms, including at least one heteroatom.

In one embodiment, each C₆₋₁₄carboaryl is independently selected from: phenyl (C₆), indanyl (C₉), indenyl (C₉), isoindenyl (C₉), naphthyl (C₁₀), azulenyl (C₁₀), tetralinyl (1,2,3,4-tetrahydronaphthalene) (C₁₀), acenaphthenyl (C₁₂), fluorenyl (C₁₃), phenalenyl (C₁₃), anthracenyl (C₁₄), and phenanthrenyl (C₁₄).

In one embodiment, each C₅₋₁₄heteroaryl is independently selected from: C₅heteroaryl groups including:
N₁: pyrrolyl (C₅);
O₁: furanyl (C₅);
S₁: thienyl (C₅);
N₁O₁: oxazolyl (C₅), isoxazolyl (C₅);
N₂O₁: oxadiazolyl (C₅);
N₃O₁: oxatriazolyl (C₅);
N₁S₁: thiazolyl (C₅), isothiazolyl (C₅);
N₂: imidazolyl (C₅), pyrazolyl (C₅);
N₃: triazolyl (C₅); and,
N₄: tetrazolyl (C₅);

C₆heteroaryl groups including:
N₁: pyridinyl (C₆);
N₁O₁: isoxazinyl (C₆);
N₂: pyridazinyl (C₆), pyrimidinyl (C₆), pyrazinyl (C₆);
N₃: triazinyl (C₆); and,

C₉heteroaryl groups including:
N₁: indolyl (C₉), isoindolyl (C₉), indolizinyl (C₉), indolinyl (C₉), isoindolinyl (C₉);
O₁: benzofuranyl (C₉), isobenzofuranyl (C₉);
S₁: benzothiofuranyl (C₉);
N₂: benzimidazolyl (C₉), indazolyl (C₉);
N₁O₁: benzoxazolyl (C₉), benzisoxazolyl (C₉).
N₁S₁: benzothiazolyl (C₉);
O₂: benzodioxolyl (C₉);
N₂O₁: benzofurazanyl (C₉);
N₂S₁: benzothiadiazolyl (C₉);
N₃: benzotriazolyl (C₉); and
N₄: purinyl (C₉);

C₁₀heteroaryl groups including:
O₁: chromenyl (C₁₀), isochromenyl (C₁₀), chromanyl (C₁₀), isochromanyl (C₁₀);
O₂: benzodioxanyl (C₁₀);
N₁: quinolinyl (C₁₀), isoquinolinyl (N₁), quinolizinyl (N₁);
N₁O₁: benzoxazinyl (C₁₀);
N₂: benzodiazinyl (C₁₀), pyridopyridinyl (C₁₀), quinoxalinyl (C₁₀), quinazolinyl (C₁₀), cinnolinyl (C₁₀), phthalazinyl C₁₀), naphthyridinyl (C₁₀); and
N₄: pteridinyl (C₁₀);

C₁₁heteroaryl groups (with 2 fused rings) including:
N₂: benzodiazepinyl (C₁₁);

C₁₃heteroaryl groups (with 3 fused rings) including:
N₁: carbazolyl (C₁₃);
O₁: dibenzofuranyl (C₁₃);
S₁: dibenzothiophenyl (C₁₃); and
N₂: carbolinyl (C₁₃), pyridoindolyl (C₁₃); and

C₁₄heterocyclic groups (with 3 fused rings) including:
N₁: acridinyl (C₁₄), phenanthridine (C₁₄);
O₁: xanthenyl (C₁₄);
S₁: thioxanthenyl (C₁₄);
N₂: phenazinyl (C₁₄), phenanthroline (C₁₄), phenazine (C₁₄);
N₁O₁: phenoxazinyl (C₁₄)′
N₁S₁: phenothiazinyl (C₁₄);
O₂: oxanthrenyl (C₁₄);
O₁S₁: phenoxathiin (C₁₄);
S₂: thianthrene (C₁₄).

### Other Forms

Unless otherwise specified, a reference to a particular group also includes the well known ionic, salt, hydrate, solvate, and protected forms thereof. For example, a reference to carboxylic acid (—COOH) also includes the anionic (carboxylate) form (—COO), a salt or hydrate or solvate thereof, as well as conventional protected forms. Similarly, a reference to an amino group includes the protonated form (—N⁺HR¹HR²), a salt or hydrate or solvate of the amino group, for example, a hydrochloride salt, as well as conventional protected forms of an amino group. Similarly, a reference to a hydroxyl group also includes the anionic form (—O⁻), a salt or hydrate or solvate thereof, as well as conventional protected forms.

### Isomers

Certain compounds may exist in one or more particular geometric, optical, enantiomeric, diastereomeric, epimeric, atropic, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

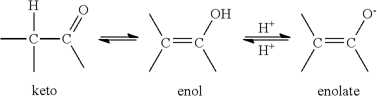

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO⁻), then a salt may be formed with a suitable cation.

Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, trifluoroacetic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Solvates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 3rd Edition; John Wiley and Sons, 1999).

Unless otherwise specified, a reference to a particular compound also includes chemically protected forms thereof.

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl (diphenylmethyl), or trityl (triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulphonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle the compound in the form of a prodrug. The term “prodrug,” as used herein, pertains to a compound which, when metabolised (e.g., in vivo), yields the desired compound. Typically, the prodrug is inactive, or less active than the compound, but may provide advantageous handling, administration, or metabolic properties.

Unless otherwise specified, a reference to a particular compound also includes prodrugs thereof.

For example, some prodrugs are esters of the compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the compound, or a compound which, upon further chemical reaction, yields the compound (for example, as in ADEPT, GDEPT, LIDEPT, etc.). For example, the prodrug may be a sugar derivative or other glycoside conjugate, or may be an amino acid ester derivative.

### Chemical Synthesis

Several methods for the chemical synthesis of compounds of the present invention are described herein. These and/or other well known methods may be modified and/or adapted in known ways in order to facilitate the synthesis of additional compounds within the scope of the present invention.

### Uses

The compounds described herein are useful, for example, in the treatment of diseases and conditions that are ameliorated by the inhibition of PKD (e.g., PKD1, PKD2, PKD3), such as, for example, proliferative conditions, cancer, etc.

Use in Methods of Inhibiting PKD

One aspect of the present invention pertains to a method of inhibiting PKD (e.g., PKD1, PKD2, PKD3) in a cell, in vitro or in vivo, comprising contacting the cell with an effective amount of a compound, as described herein.

Suitable assays for determining PKD (e.g., PKD1, PKD2, PKD3) inhibition are known in the art and/or are described herein.

Use in Methods of Inhibiting Cell Proliferation, Etc.

The compounds described herein, e.g., (a) regulate (e.g., inhibit) cell proliferation; (b) inhibit cell cycle progression; (c) promote apoptosis; or (d) a combination of one or more of these.

One aspect of the present invention pertains to a method of regulating (e.g., inhibiting) cell proliferation (e.g., proliferation of a cell), inhibiting cell cycle progression, promoting apoptosis, or a combination of one or more these, in vitro or in vivo, comprising contacting cells (or the cell) with an effective amount of a compound, as described herein.

In one embodiment, the method is a method of regulating (e.g., inhibiting) cell proliferation (e.g., proliferation of a cell), in vitro or in vivo, comprising contacting cells (or the cell) with an effective amount of a compound, as described herein.

In one embodiment, the method is performed in vitro.

In one embodiment, the method is performed in viva

In one embodiment, the compound is provided in the form of a pharmaceutically acceptable composition.

Any type of cell may be treated, including but not limited to, lung, gastrointestinal (including, e.g., bowel, colon), breast (mammary), ovarian, prostate, liver (hepatic), kidney (renal), bladder, pancreas, brain, and skin.

One of ordinary skill in the art is readily able to determine whether or not a candidate compound regulates (e.g., inhibits) cell proliferation, etc. For example, assays which may conveniently be used to assess the activity offered by a particular compound are described herein.

For example, a sample of cells (e.g., from a tumour) may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a patient carrying cells of the same cellular type.

### Use in Methods of Therapy

Another aspect of the present invention pertains to a compound as described herein for use in a method of treatment of the human or animal body by therapy.

### Use in the Manufacture of Medicaments

Another aspect of the present invention pertains to use of a compound, as described herein, in the manufacture of a medicament for use in treatment.

In one embodiment, the medicament comprises the compound.

### Methods of Treatment

Another aspect of the present invention pertains to a method of treatment comprising administering to a patient in need of treatment a therapeutically effective amount of a compound as described herein, preferably in the form of a pharmaceutical composition.

### Conditions Treated—Conditions Mediated by PKD

In one embodiment (e.g., of use in methods of therapy, of use in the manufacture of medicaments, of methods of treatment), the treatment is treatment of a disease or condition that is mediated by PKD (e.g., PKD1, PKD2, PKD3).

### Conditions Treated—Conditions Ameliorated by the Inhibition of PKD

In one embodiment (e.g., of use in methods of therapy, of use in the manufacture of medicaments, of methods of treatment), the treatment is treatment of: a disease or condition that is ameliorated by the inhibition of PKD (e.g., PKD1, PKD2, PKD3).

### Conditions Treated—Proliferative Conditions and Cancer

In one embodiment (e.g., of use in methods of therapy, of use in the manufacture of medicaments, of methods of treatment), the treatment is treatment of: a proliferative condition.

The term “proliferative condition,” as used herein, pertains to an unwanted or uncontrolled cellular proliferation of excessive or abnormal cells which is undesired, such as, neoplastic or hyperplastic growth.

In one embodiment, the treatment is treatment of: a proliferative condition characterised by benign, pre-malignant, or malignant cellular proliferation, including but not limited to, neoplasms, hyperplasias, and tumours (e.g., histocytoma, glioma, astrocyoma, osteoma), cancers (see below), psoriasis, bone diseases, fibroproliferative disorders (e.g., of connective tissues), pulmonary fibrosis, atherosclerosis, smooth muscle cell proliferation in the blood vessels, such as stenosis or restenosis following angioplasty.

In one embodiment, the treatment is treatment of: cancer.

In one embodiment, the treatment is treatment of: lung cancer, small cell lung cancer, non-small cell lung cancer, gastrointestinal cancer, stomach cancer, bowel cancer, colon cancer, rectal cancer, colorectal cancer, thyroid cancer, breast cancer, ovarian cancer, endometrial cancer, prostate cancer, testicular cancer, liver cancer, kidney cancer, renal cell carcinoma, bladder cancer, pancreatic cancer, brain cancer, glioma, sarcoma, osteosarcoma, bone cancer, skin cancer, squamous cancer, Kaposi's sarcoma, melanoma, malignant melanoma, lymphoma, or leukemia.

In one embodiment, the treatment is treatment of:
a carcinoma, for example a carcinoma of the bladder, breast, colon (e.g., colorectal carcinomas such as colon adenocarcinoma and colon adenoma), kidney, epidermal, liver, lung (e.g., adenocarcinoma, small cell lung cancer and non-small cell lung carcinomas), oesophagus, gall bladder, ovary, pancreas (e.g., exocrine pancreatic carcinoma), stomach, cervix, thyroid, prostate, skin (e.g., squamous cell carcinoma);
a hematopoietic tumour of lymphoid lineage, for example leukemia, acute lymphocytic leukemia, B-cell lymphoma, T-cell lymphoma, Hodgkin's lymphoma, non-Hodgkin's lymphoma, hairy cell lymphoma, or Burkett's lymphoma;
a hematopoietic tumor of myeloid lineage, for example acute and chronic myelogenous leukemias, myelodysplastic syndrome, or promyelocytic leukemia;
a tumour of mesenchymal origin, for example fibrosarcoma or habdomyosarcoma;
a tumor of the central or peripheral nervous system, for example astrocytoma, neuroblastoma, glioma or schwannoma;
melanoma; seminoma; teratocarcinoma; osteosarcoma; xenoderoma pigmentoum; keratoctanthoma; thyroid follicular cancer; or Kaposi's sarcoma.

In one embodiment, the treatment is treatment of solid tumour cancer.

The anti-cancer effect may arise through one or more mechanisms, including but not limited to, the regulation of cell proliferation, the inhibition of cell cycle progression, the inhibition of angiogenesis (the formation of new blood vessels), the inhibition of metastasis (the spread of a tumour from its origin), the inhibition of invasion (the spread of tumour cells into neighbouring normal structures), or the promotion of apoptosis (programmed cell death). The compounds of the present invention may be used in the treatment of the cancers described herein, independent of the mechanisms discussed herein.

In one embodiment (e.g., of use in methods of therapy, of use in the manufacture of medicaments, of methods of treatment), the treatment is treatment of: a hyperproliferative skin disorder.

In one embodiment, the treatment is treatment of: psoriasis, actinic keratosis, and/or non-melanoma skin cancer.

### Conditions Treated—Conditions Characterised by Pathological Angiogenesis

In one embodiment (e.g., of use in methods of therapy, of use in the manufacture of medicaments, of methods of treatment), the treatment is treatment of: a disease or condition that is characterised by inappropriate, excessive, and/or undesirable angiogenesis (as “anti-angiogenesis agents”).

Examples of such conditions include macular degeneration, cancer (solid tumours), psoriasis, and obesity.

### Conditions Treated—Inflammation etc.

In one embodiment (e.g., of use in methods of therapy, of use in the manufacture of medicaments, of methods of treatment), the treatment is treatment of: an inflammatory disease.

In one embodiment, the treatment is treatment of: an inflammatory disease involving pathological activation of T- and B-cell lymphocytes, neutrophils, and/or Mast cells.

In one embodiment, the treatment is treatment of: an inflammatory disease, such as rheumatoid arthritis, osteoarthritis, rheumatoid spondylitis, gouty arthritis, traumatic arthritis, rubella arthritis, psoriatic arthritis, and other arthritic conditions; Alzheimer's disease; toxic shock syndrome, the inflammatory reaction induced by endotoxin or inflammatory bowel disease; tuberculosis; atherosclerosis; muscle degeneration; Reiter's syndrome; gout; acute synovitis; sepsis; septic shock; endotoxic shock; gram negative sepsis; adult respiratory distress syndrome; cerebral malaria; chronic pulmonary inflammatory disease; silicosis; pulmonary sarcoisosis; bone resorption diseases; reperfusion injury; graft versus host reaction; allograft rejections; fever and myalgias due to infection, such as influenza, cachexia, in particular cachexia secondary to infection or malignancy, cachexia secondary to acquired immune deficiency syndrome (AIDS); AIDS; ARC (AIDS related complex); keloid formation; scar tissue formation; Crohn's disease; ulcerative colitis; pyresis; chronic obstructive pulmonary disease (COPD); acute respiratory distress syndrome (ARDS); asthma; pulmonary fibrosis; bacterial pneumonia.

In one preferred embodiment, the treatment is treatment of: an arthritic condition, including rheumatoid arthritis and rheumatoid spondylitis; inflammatory bowel disease, including Crohn's disease and ulcerative colitis; and chronic obstructive pulmonary disease (COPD).

In one preferred embodiment, the treatment is treatment of: an inflammatory disorder characterized by T-cell proliferation (T-cell activation and growth), for example, tissue graft rejection, endotoxin shock, and glomerular nephritis.

### Conditions Treated—Heart Failure

The compounds of the present invention are useful in the treatment of conditions associated with heart remodelling.

In one embodiment, the treatment is treatment of: myocyte hypertrophy of the heart, impaired contractility of the heart, and/or pump failure of the heart.

In one embodiment, the treatment is treatment of: pathologic cardiac hypertrophy.

In one embodiment, the treatment is treatment of: heart failure.

### Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, alleviation of symptoms of the condition, amelioration of the condition, and cure of the condition. Treatment as a prophylactic measure (i.e., prophylaxis) is also included. For example, use with patients who have not yet developed the condition, but who are at risk of developing the condition, is encompassed by the term “treatment.”

For example, treatment includes the prophylaxis of cancer, reducing the incidence of cancer, alleviating the symptoms of cancer, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents, for example, cytotoxic agents, anticancer agents, etc. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

For example, it may be beneficial to combine treatment with a compound as described herein with one or more other (e.g., 1, 2, 3, 4) agents or therapies that regulates cell growth or survival or differentiation via a different mechanism, thus treating several characteristic features of cancer development.

One aspect of the present invention pertains to a compound as described herein, in combination with one or more additional therapeutic agents, as described below.

The particular combination would be at the discretion of the physician who would select dosages using his common general knowledge and dosing regimens known to a skilled practitioner.

The agents (i.e., the compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the compound described here, plus one or more other agents) may be formulated together in a single dosage form, or alternatively, the individual agents may be formulated separately and presented together in the form of a kit, optionally with instructions for their use.

### Other Uses

The compounds described herein may also be used as cell culture additives to inhibit PKD (e.g., PKD1, PKD2, PKD3), to inhibit cell proliferation, etc.

The compounds described herein may also be used as part of an in vitro assay, for example, in order to determine whether a candidate host is likely to benefit from treatment with the compound in question.

The compounds described herein may also be used as a standard, for example, in an assay, in order to identify other compounds, other PKD (e.g., PKD1, PKD2, PKD3) inhibitors, other anti-proliferative agents, other anti-cancer agents, etc.

### Kits

One aspect of the invention pertains to a kit comprising (a) a compound as described herein, or a composition comprising a compound as described herein, e.g., preferably provided in a suitable container and/or with suitable packaging; and (b) instructions for use, e.g., written instructions on how to administer the compound or composition.

The written instructions may also include a list of indications for which the active ingredient is a suitable treatment.

### Routes of Administration

The compound or pharmaceutical composition comprising the compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the compound to be administered alone, it is preferable to present it as a pharmaceutical formulation (e.g., composition, preparation, medicament) comprising at least one compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 2nd edition, 1994.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

Formulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additional contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient. Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection. Typically, the concentration of the compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the compounds, and compositions comprising the compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the patient. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the compound is in the range of about 100 μg to about 250 mg (more typically about 100 μg to about 25 mg) per kilogram body weight of the subject per day. Where the compound is a salt, an ester, an amide, a prodrug, or the like, the amount administered is calculated on the basis of the parent compound and so the actual weight to be used is increased proportionately.

### EXAMPLES

The following examples are provided solely to illustrate the present invention and are not intended to limit the scope of the invention, as described herein.

### Synthesis Examples

### General Methods

### Reagents

All reagents were commercial grade and were used as received without further purification, unless otherwise specified. Commercially available anhydrous solvents were used for reactions conducted under inert atmosphere. Reagent grade solvents were used in all other cases, unless otherwise specified.

### General Methods: Chromatography

Column chromatography was performed on Isolute® Flash Si II silica cartridges or Silica 60A (particle size 35-70 micron) from Fisher Scientific. Thin layer chromatography was carried out using pre-coated silica gel F-254 plates (thickness 0.25 mm).

### General Methods: NMR

¹H NMR spectra were recorded on a Bruker DPX400 at 400 MHz. Chemical shifts for ¹H NMR spectra are given in parts per million and either tetramethylsilane (0.00 ppm) or residual solvent peaks were used as internal reference. Splitting patterns are designated as follows: s, singlet; d, doublet; t, triplet; q, quartet; p, pentet; m, multiplet; bs, broad singlet. Electrospray MS spectra were obtained on either a Micromass Platform or ZQ spectrometer.

### General Methods: LCMS Methods

Samples analysed by Liquid Chromatography-Mass Spectrometry (LCMS) and employed standard conditions (method A (acidic), method B (basic)) as described below.

### Method A: Acidic LC-MS Conditions (10 cm Formic Mode)

### HPLC Setup:

### Solvents:

Acetonitrile (Far UV grade) with 0.1% (V/V) formic acid.

Water (High purity via Elga UHQ unit) with 0.1% formic acid.

### Column:

Phenomenex Luna 5 μm C18 (2), 100×4.6 mm. (Plus guard cartridge).

### Flow Rate:

2 mL/minute.

### Gradient:

A: Water/formic.

B: MeCN/formic.

| Time | A % | B % |
|---|---|---|
| 0.00 | 95 | 5 |
| 3.50 | 5 | 95 |
| 5.50 | 5 | 95 |
| 5.60 | 95 | 5 |
| 6.50 | 95 | 5 |

### Typical Injections:

2-7 μL.

### UV Detection via HP or Waters DAD:

Start Range (nm): 210.

End Range (nm): 400.

Range interval (nm): 4.0.

Other wavelength traces are extracted from the DAD data.

Optional ELS detection using Polymer Labs ELS-1000.

### MS Detection:

Either Micromass Platform or ZQ, both single quadrapole LC-MS instruments.

Flow splitter gives approximately 300 μL/minute to mass spectrometer.

Scan Range for MS Data (m/z):

Start (m/z): 100.

End (m/z): 650, or 1000 when required.

With +ve/−ve switching.

Ionisation is either electrospray or APCI dependent on compound types (the ZQ has an ESCI option which can give both ESI and APCI data from a single run).

### Typical ESI Voltages and Temperatures are:

Source 120-150° C.

3.5 kV capillary

25 V cone.

### Typical APCI Voltages and Temperatures are:

Source 140-160° C.

17 μA corona.

25 V cone.

Desolvation (Platform): 350° C.

### Method B: Basic LC-MS Conditions (10 cm Ammonia or 10 cm Bicarb Modes)

### HPLC Setup

### Solvents:

10 cm ammonia mode:

Acetonitrile with 7 mM ammonia (Far UV grade).

Water (high purity via Elga UHQ unit) with 7 mM ammonia.

### 10 cm Bicarb Mode:

Acetonitrile (far UV grade).

Water (high purity via Elga UHQ unit) with 10 mM ammonium bicarbonate.

### Column:

Waters Xterra MS 5 μm C18, 100×4.6 mm. (Plus guard cartridge).

### Flow Rate:

2 mL/minute.

### Gradient:

10 cm ammonia mode:

A: Water/Ammonia.

B: MeCN/Ammonia.

### 10 cm Bicarb Mode:

A: Water/Ammonium bicarbonate.

B: MeCN

| Time | A % | B % |
|---|---|---|
| 0.00 | 95 | 5 |
| 3.50 | 5 | 95 |
| 5.50 | 5 | 95 |
| 5.60 | 95 | 5 |
| 6.50 | 95 | 5 |

### Typical Injections:

2-7 μL (concentration about 0.1-1 mg/mL).

### UV Detection Via HP or Waters DAD

Start Range (nm): 210.

End Range (nm): 400.

Range interval (nm): 4.0.

Other wavelength traces are extracted from the DAD data.

Optional ELS detection using Polymer Labs ELS-1000.

### MS Detection:

Either Micromass Platform or ZQ, both single quadrapole LC-MS instruments.

Flow splitter gives approximately 300 μL/minutes to mass spectrometer.

Scan Range for MS Data (m/z):

Start (m/z): 100.

End (m/z): 650, or 1000 when required.

With +ve/−ve switching.

Ionisation is either electrospray or APCI dependent on compound types (the ZQ has an ESCI option which can give both ESI and APCI data from a single run).

### Typical ESI Voltages and Temperatures are:

Source 120-150° C.

3.5 kV capillary.

25 V cone.

### Typical APCI Voltages and Temperatures are:

Source 140-160° C.

17 μA corona.

25 V cone.

Desolvation (Platform) 350° C.

### Synthesis of Boronate Esters

Boronate esters that were not commercially available were synthesised from the appropriate aryl bromides following the method illustrated in the following scheme.

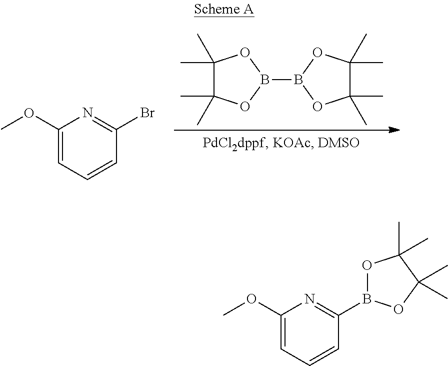

### Synthesis A

### 2-Methoxy-6-(4,4,5,5-tetramethyl-[1,3,2]dioxaborolan-2-yl)-pyridine

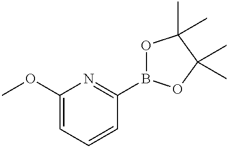

To a mixture of 2-methoxy-6-bromopyridine (374 mg; 2.0 mmol) and bis(pinacolato) diboron (1.1 g; 4.0 mmol) in dimethylsulfoxide (degassed, 10 mL) was added[1,1′-bis(diphenylphosphino)ferrocene]dichloropalladium(II) (82 mg; 0.1 mmol) and potassium acetate (0.60 g; 6.0 mmol). The mixture was heated at 80° C. under a nitrogen atmosphere for 2 hours. The reaction mixture was poured into water (150 mL) with stirring to give a dark precipitate, which was collected by filtration, washed with water, and dried under reduced pressure. The crude product was used without further purification. ¹H (400 MHz, CDCl₃) 7.57 (1H, dd), 7.44 (1H, d), 6.78 (1H, d), 4.02 (3H, s), 1.36 (12H, s).

In other cases, the boronate ester was isolated by extraction of the aqueous mixture obtained during work-up with ethyl acetate, followed by concentration of the organic or aqueous phase.

The following boronate esters were synthesised in an analogous manner starting from the appropriate aryl bromides:

| # | Name | Structure |
|---|---|---|
| 1 | 5-(4,4,5,5-Tetramethyl- [1,3,2]dioxaborolan-2-yl)-1H- indazole | 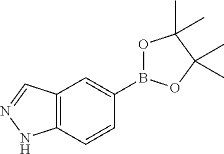 |
| 2 | 2-(6-Ethoxy-naphthalen-2-yl)- 4,4,5,5-tetramethyl- [1,3,2]dioxaborolane | 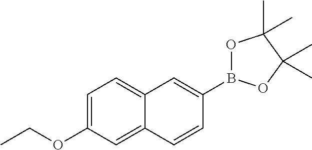 |
| 3 | N-(2-Diethylamino-ethyl)-3- (4,4,5,5-tetramethyl- [1,3,2]dioxaborolan-2-yl)- benzamide | 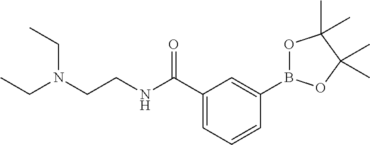 |

### General Synthesis Procedure A

Compounds were synthesised starting from 2,6-dichloropyrazine following the scheme illustrated below.

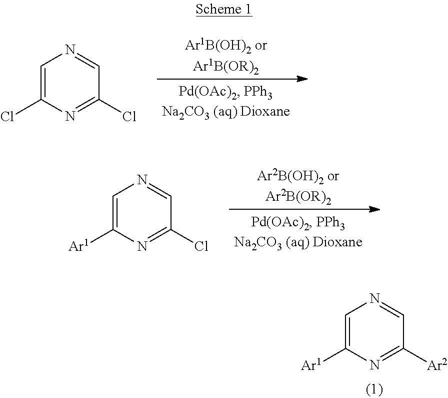

### Synthesis 1

### 4-(6-Chloro-pyrazin-2-yl)-2-methoxyphenol

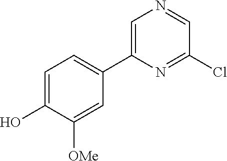

To a solution of 2,6-dichloropyrazine (200 mg; 1.35 mmol), 2-methoxy-4-(4,4,5,5-tetramethyl-[1,3,2]dioxaborolan-2-yl)-phenol (400 mg; 1.62 mmol) in dioxane (2 mL) was added palladium (II) acetate (15 mg; 0.07 mmol) and triphenylphosphine (60 mg; 0.22 mmol). Aqueous sodium carbonate (1 mL; 1.5 M) was added and the mixture was heated in a Smith Creator® microwave at 140° C. for 600 seconds. The reaction was partitioned between ethyl acetate (10 mL) and water (8 mL). The organic phase was separated, dried over MgSO₄, filtered, and evaporated to give a yellow oil. The crude product was chromatographed on silica eluted with dichloromethane followed by diethyl ether to elute the product. Appropriate fractions were pooled and evaporated to give the title compound as a white solid (92 mg). ¹H (400 MHz, CDCl₃) 8.87 (1H, s), 8.44 (1H, s), 7.62 (1H, d), 7.54 (1H, dd), 7.03 (1H, d), 4.02 (3H, s). LCMS (method B) R_{T} 2.46, MI 237:239 (3:1 isotopes, M+H⁺).

### Synthesis 2

### N-(2-Dimethylamino-ethyl)-3-[6-(4-hydroxy-3-methoxy-phenyl)-pyrazin-2-yl]-benzamide

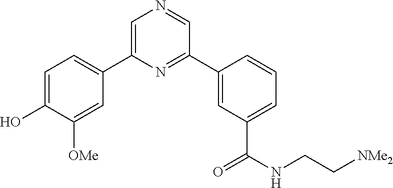

To a solution of 4-(6-chloro-pyrazin-2-yl)-2-methoxyphenol (50 mg; 0.21 mmol) and N-(2-dimethylamino-ethyl)-3-(4,4,5,5-tetramethyl-[1,3,2]dioxaborolan-2-yl)-benzamide (53 mg; 0.21 mmol) in dioxane (1 mL) was added palladium (II) acetate (4 mg; 0.016 mmol) and triphenylphosphine (17 mg; 0.07 mmol). Aqueous sodium carbonate (0.5 mL; 1.5 M) was added and the mixture heated in a Smith Creator@ microwave at 140° C. for 600 seconds. The reaction was partitioned between ethyl acetate (10 mL) and water (5 mL). The organic phase was separated, dried over MgSO₄, filtered, and evaporated to give a yellow oil. The crude product was chromatographed on silica eluted with dichloromethane followed by 10% methanol in dichloromethane containing 0.1% NH₄OH to elute the product. Appropriate fractions were pooled and evaporated to give an oil which crystallised from ethyl acetate on standing to give the title compound as a pale yellow solid (29 mg). ¹H (400 MHz, CDCl₃) 8.90 (2H, d), 8.50 (1H, s), 8.25 (1H, m), 7.89 (1H, d), 7.68 (1H, s), 7.62 (2H, m), 7.01 (2H, m), 4.00 (3H, s), 3.60 (2H, q), 2.58 (2H, q), 2.31 (6H, s). LCMS (method A) R_{T} 2.19, MI 393 (M+H⁺).

The following compounds were synthesised in an analogous manner using General Synthesis Procedure A.

| | Analytical LCMS | | |
|---|---|---|---|
| ID No. | retention time (min) | MI + H | LCMS method |
| X-001 | 1.65 | 391 | 10 cm-apci-formic |
| X-002 | 1.61 | 377 | 10 cm-apci-formic |
| X-003 | 2.32 | 437 | 10 cm-apci-formic |
| X-004 | 1.10 | 377 | 10 cm-apci-formic |
| X-005 | 1.91 | 377 | 10 cm-apci-formic |
| X-006 | 1.97 | 418 | 10 cm-apci-formic |
| X-007 | 2.19 | 393 | 10 cm-esci-NH₃-DA |
| X-013 | 2.58 | 437 | 10 cm-apci-formic-DA |
| X-014 | 2.66 | 327 | 10 cm-esci-ammonia |
| X-018 | 2.72 | 322 | 10 cm-apci-formic-DA |
| X-020 | 3.21 | 364 | 10 cm-apci-formic |
| X-021 | 2.12 | 365 | 10 cm-apci-formic |

### General Synthesis Procedure B

Compounds were synthesised starting from 4-(6-chloro-pyrazin-2-yl)-2-methoxyphenol 2,6-dichloropyrazine (described above), following the scheme illustrated below.

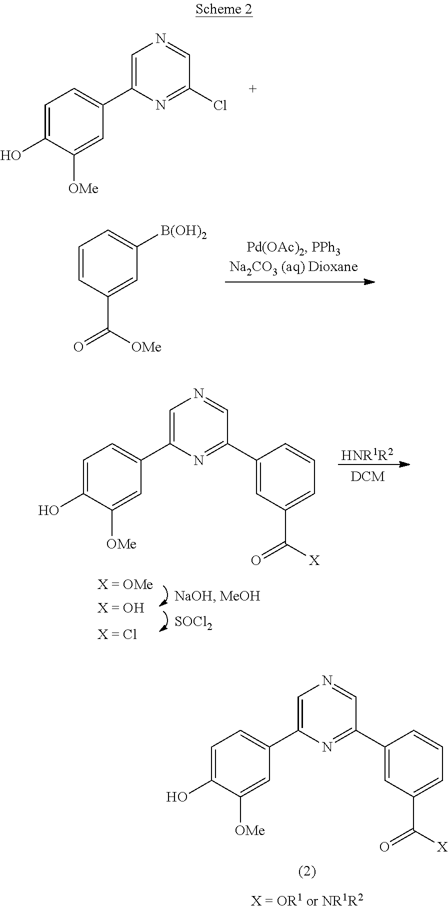

### Synthesis 3

### 3-[6-(4-Hydroxy-3-methoxy-phenyl)-pyrazin-2-yl]-benzoic acid methyl ester

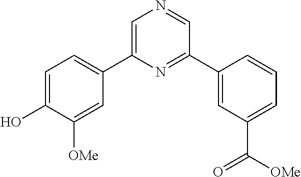

To a solution of 4-(6-chloro-pyrazin-2-yl)-2-methoxyphenol (300 mg; 1.26 mmol) and 3-methyoxycarbonylphenyl boronic acid (230 mg; 1.26 mmol) in dioxane (3 mL) was added palladium (II) acetate (15 mg; 0.07 mmol) and triphenylphosphine (60 mg; 0.22 mmol). Aqueous sodium carbonate (1.5 mL; 1.5 M) was added and the mixture heated in the Smith Creator® microwave at 80° C. for 30 minutes. The reaction was partitioned between ethyl acetate (30 mL) and water (20 mL). The organic phase was separated, dried over MgSO₄, filtered, and evaporated to give a pale yellow powder. The crude product was chromatographed on silica eluted with 1:1 ethyl acetate:hexane. Appropriate fractions were pooled and evaporated to give an oil which crystallised from ethyl acetate on standing to give the title compound as a pale yellow solid (250 mg). ¹H (400 MHz, CDCl₃) 8.95 (2H, s), 8.78 (1H, s), 8.36 (1H, d), 8.17 (1H, d), 7.77 (1H, s), 7.65 (2H, m), 7.07 (1H, d), 5.90 (1H, s), 4.05 (3H, s), 3.99 (3H, s). LCMS (method A) R_{T} 3.65, MI 337 (M+H⁺).

### Synthesis 4

### 3-[6-(4-Hydroxy-3-methoxy-phenyl)-pyrazin-2-yl]-benzoic acid

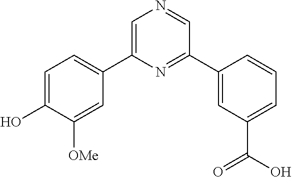

To a suspension of 3-[6-(4-hydroxy-3-methoxy-phenyl)-pyrazin-2-yl]-benzoic acid methyl ester (250 mg; 0.74 mmol) in methanol (20 mL) was added sodium hydroxide (2 mL; 2N) and the solution was stirred for 36 hours. The mixture was acidified by the addition of hydrochloric acid and the precipitate collected by filtration, washing with water then dried under vacuum to give the title compound as a red solid (240 mg). ¹H (400 MHz, d6-DMSO) 13.23 (1H, bs), 9.65 (1H, bs), 9.24 (1H, s), 9.18 (1H, d), 8.79 (1H, s), 8.51 (1H, d), 8.12 (1H, d), 7.85 (1H, s), 7.74 (2H, m), 7.02 (1H, d), 3.94 (3H, s). LCMS (method A) R_{T}=3.05, MI 323 (M+H⁺).

### Synthesis 5

### 3-[6-(4-Hydroxy-3-methoxy-phenyl)-pyrazin-2-yl]-N-isopropyl-benzamide

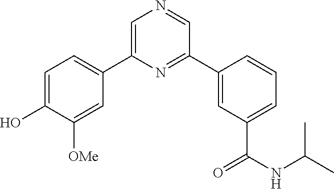

A suspension of 3-[6-(4-hydroxy-3-methoxy-phenyl)-pyrazin-2-yl]-benzoic acid (20 mg; 0.06 mmol) in thionyl chloride (2 mL) was heated at reflux for 30 minutes and then volatile material removed under vacuum and the flask flushed with dry nitrogen. The residue was suspended in tetrahydrofuran (2 mL), a solution of isopropyl amine (0.5 mL) in tetrahydrofuran (2 mL) was added, and the reaction was stirred for 3 hours. The solvent was removed under vacuum and the residue was purified by preparative HPLC (method B) to give the title compound as a colourless powder (4 mg). ¹H (400 MHz, CDCl₃) 8.95 (2H, s), 8.51 (1H, s), 8.25 (1H, d), 7.85 (1H, d), 7.75 (1H, s), 7.62 (2H, m), 7.07 (1H, d), 5.90 (1H, bs), 5.86 (1H, bs), 4.34 (1H, m), 4.04 (3H, s), 1.30 (6H, d). LCMS (method A) R_{T}=3.21, MI 364 (M+H⁺).

The following compounds were synthesised in an analogous manner using General Synthesis Procedure B.

Analytical LCMS

| ID No. | retention time (min) | MI + H | LCMS method |
|---|---|---|---|
| X-009 | 2.92 | 392 | 10 cm-apci-formic |
| X-010 | 2.04 | 391 | 10 cm-apci-formic |
| X-011 | 3.12 | 376 | 10 cm-apci-formic |
| X-019 | 2.07 | 419 | 10 cm-apci-formic |
| X-022 | 2.11 | 421 | 10 cm-apci-formic |

### General Synthesis Procedure C

Compounds were synthesised starting from 2-aminopyrazine following the scheme illustrated below.

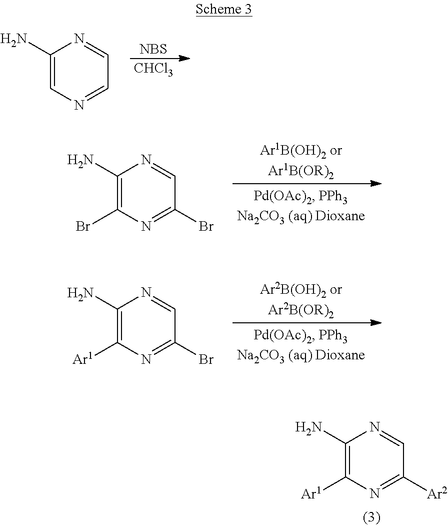

### Synthesis 6

### 2-Amino-3,5-dibromopyrazine

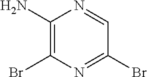

Aminopyrazine (95 g, 1 mol) in chloroform (2.5 L) was cooled to between 0° C. and -5° C. N-bromosuccimide (375 g, 2.1 mol) was then added over a 6 hour period, during which time the temperature was kept below 0° C. The reaction mixture was stored in a freezer overnight and then stirred vigorously and quenched with water (1 L). The mixture was filtered through glass wool and the phases separated. The organic phase was washed with 10% K₂CO₃ (aq, 1 L), dried over MgSO₄, and concentrated in vacuo. The residue was triturated with hexane and ethyl acetate. The yellow/brown solid was filtered and dried under high vacuum in a desiccator overnight to give the title compound (119 g). ¹H (270 MHz, CDCl₃) 4.99 (2H, bs), 8.09 (1H, s); LC-MS (AP⁺): 254:256 (1:1 isotopes, M+H⁺).

### Synthesis 7

### 5-Bromo-3-(2-methoxy-phenyl)-pyrazin-2-ylamine

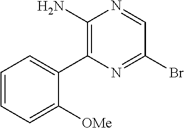

To a solution of 2-amino-3,5-dibromopyrazine (400 mg; 1.59 mmol), and 2-methyoxyphenylboronic acid (242 mg; 1.59 mmol) in dioxane (4 mL) was added palladium (II) acetate (17.8 mg; 0.08 mmol) and triphenylphosphine (83.5 mg; 0.31 mmol). Aqueous sodium carbonate (1 mL; 1.5 M) was added and the mixture heated in the Smith Creator® microwave at 130° C. for 800 seconds. The reaction mixture was diluted with dichloromethane (6 mL) and water (3 mL) and poured through a PTFE separation frit. The dichloromethane filtrate was collected and evaporated. The crude product was chromatographed on silica eluted on a gradient from 20 to 50% diethyl ether in petroleum ether (40-60° C. fraction). Appropriate fractions were pooled and evaporated to give the title compound as a white solid (290 mg). ¹H (400 MHz, CDCl₃) 8.10 (1H, s), 7.43 (2H, m), 7.10 (1H, m), 7.01 (1H, d), 4.68 (2H, bs), 3.86 (3H, s). LCMS (method B) R_{T}=3.19, MI 280:282 (isotopes; M+H⁺).

### Synthesis 8

### 3-[5-Amino-6-(2-methoxy-phenyl)-pyrazin-2-yl]-N-(2-dimethylamino-ethyl)-benzamide

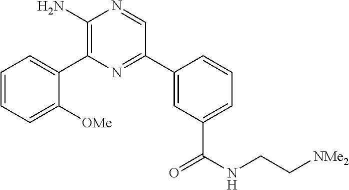

To a solution of 5-bromo-3-(2-methoxy-phenyl)-pyrazin-2-ylamine (50 mg; 0.178 mmol), and N-(2-dimethylamino-ethyl)-3-(4,4,5,5-tetramethyl-[1,3,2]dioxaborolan-2-yl)-benzamide (44 mg; 1.78 mmol) in dioxane (1 mL) was added palladium (II) acetate (2 mg; 0.009 mmol) and triphenylphosphine (9 mg; 0.035 mmol). Aqueous sodium carbonate (0.5 mL; 1.5 M) was added and the mixture heated in a Smith Creator® microwave at 130° C. for 800 seconds. The reaction mixture was diluted with dichloromethane (6 mL) and water (2 mL) and poured through a PTFE separation frit. The dichloromethane filtrate was collected and evaporated. The crude product was chromatographed on silica eluted with dichloromethane followed by 10% methanol in dichloromethane containing 0.1% NH₄OH to elute the product. Appropriate fractions were pooled and evaporated to give the title compound as a pale yellow solid (23 mg). ¹H (400 MHz, CDCl₃) 8.54 (1H, s), 8.35 (1H, s), 8.08 (1H, d), 7.75 (1H, d), 7.49 (3H, m), 7.12 (1H, t), 7.06 (1H, d), 6.91 (1H, bs), 4.78 (2H, bs), 3.88 (3H, s), 3.56 (2H, q), 2.56 (2H, q), 2.29 (6H, s). LCMS (method B) R_{T}=3.15, MI 392 (M+H⁺).

The following compounds were synthesised in an analogous manner using General Synthesis Procedure C.

Analytical LCMS

| ID No. | retention time (min) | MI + H | LCMS method |
|---|---|---|---|
| X-008 | 3.30 | 464 | 10 cm-esci-ammonia |
| X-012 | 2.39 | 452 | 10 cm-apci-formic |
| X-015 | 3.15 | 392 | 10 cm-esci-ammonia |
| X-016 | 2.15 | 410 | 10 cm-apci-formic |
| X-017 | 2.00 | 408 | 10 cm-apci-formic |
| X-027 | 2.26 | 420 | 10 cm-apci-formic |

### General Synthesis Procedure D

Compounds were synthesised starting from 2-amino-5-bromo-pyrazine following the scheme illustrated below.

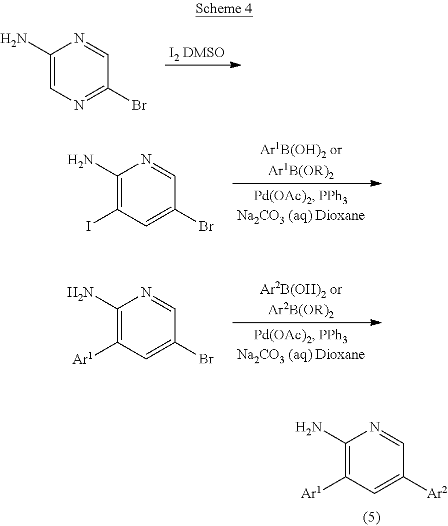

### Synthesis 9

### 5-Bromo-3-iodo-pyridin-2-ylamine

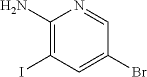

A solution of 5-bromopyridin-2-ylamine (1.73 g, 0.01 M) in dimethylsulfoxide (10 mL) was treated with iodine (3.05 g, 0.012 M) and the resulting mixture was stirred at 100° C. for 4 hours. After standing at room temperature overnight the mixture was poured onto sodium metabisulfite solution and extracted with ethyl acetate (twice). The combined extracts were dried and evaporated in vacuo. The resulting crude product was purified by flash silica chromatography, eluting with a 1:1 mixture of 40/60 petrol ether:ethyl acetate, to give the title compound as a cream coloured solid (1.12 g). ¹H-NMR (400 MHz, d6-DMSO): 8.08 (1H, s), 8.04 (1H, s), 6.30 (2H, s).

### Synthesis 10

### 5-Bromo-3-(3,4-dimethoxy-phenyl)-pyridin-2-ylamine

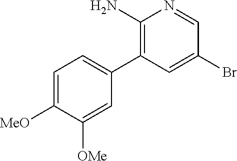

To a degassed mixture of 5-bromo-3-iodo-pyridin-2-ylamine (1.0 g; 4 mmol), 3,4-dimethoxybenzeneboronic acid (767 mg; 4.2 mmol) in dioxane (8 mL) and aqueous sodium carbonate (2 mL; 1.5 M) was added palladium (II) acetate (45 mg; 0.2 mmol) and triphenylphosphine (210 mg; 0.8 mmol) and the mixture heated at reflux for 18 hours. The reaction was partitioned between ethyl acetate (30 mL) and water (15 mL). The organic phase was separated, dried over MgSO₄, filtered, and evaporated to give a brown solid. The crude product was chromatographed on silica eluted with DCM followed by 1% methanol in dichloromethane. Appropriate fractions were pooled and evaporated to give the title compound as a cream coloured solid (723 mg). ¹H (400 MHz, CDCl₃) 8.08 (1H, s), 7.46 (1H, s), 6.79 (3H, m), 4.61 (2H, bs), 3.93 (3H, s), 3.91 (3H, s). LCMS (method B) R_{T}=3.27, MI 309:311 (1:1 isotopes M+H⁺).

### Synthesis 11

### 3-[6-Amino-5-(3,4-dimethoxy-phenyl)-pyridin-3-yl]-benzamide

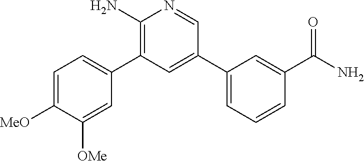

To a solution of 5-bromo-3-(3,4-dimethoxy-phenyl)-pyridin-2-ylamine (80 mg; 0.26 mmol), and 3-aminocarbonylbenzene boronic acid (454 mg; 0.27 mmol) in dioxane (1 mL) was added palladium (II) acetate (3 mg; 0.0013 mmol) and triphenylphosphine (14 mg; 0.051 mmol). Aqueous sodium carbonate (0.5 mL; 1.5 M) was added and the mixture heated in a Smith Creator® microwave at 130° C. for 800 seconds. The reaction was diluted with dichloromethane (6 mL) and water (2 mL) and poured through a PTFE separation frit. The dichloromethane filtrate was collected and evaporated. The crude product was chromatographed on silica eluted with dichloromethane followed by 8% methanol in dichloromethane to elute the product. Appropriate fractions were pooled and evaporated to give the title compound as a cream coloured solid (25 mg). ¹H (400 MHz, CD₃OD) 8.31 (1H, s), 8.15 (1H, s), 7.84 (3H, m), 7.57 (1H, t), 7.12 (3H, m), 3.90 (6H, s). LCMS (method B) R_{T} 2.71, MI 350 (M+H⁺).

The following compounds were synthesised in an analogous manner using General Synthesis Procedure D.

Analytical LCMS

| ID No. | retention time (min) | MI + H | LCMS method |
|---|---|---|---|
| Y-001 | 1.96 | 336 | 10 cm-apci-formic |
| Y-002 | 2.83 | 320 | 10 cm-esci-ammonia |
| Y-003 | 2.16 | 403 | 10 cm-apci-formic |
| Y-004 | 1.51 | 419 | 10 cm-apci-formic |
| Y-009 | 2.91 | 334 | 10 cm-esci-ammonia |
| Y-010 | 3.08 | 405 | 10 cm-esci-ammonia |
| Y-016 | 2.77 | 280 | 10 cm-esci-ammonia |
| Y-017 | 2.99 | 351 | 10 cm-esci-ammonia |
| Y-019 | 1.93 | 411 | 10 cm-apci-formic |
| Y-021 | 2.58 | 341 | 10 cm-esci-ammonia |
| Y-022 | 2.88 | 412 | 10 cm-esci-ammonia |
| Y-023 | 1.68 | 412 | 10 cm-apci-formic |
| Y-025 | 2.03 | 329 | 10 cm-esci-ammonia |
| Y-026 | 1.75 | 400 | 10 cm-apci-formic |
| Y-034 | 2.71 | 350 | 10 cm-esci-ammonia |
| Y-035 | 3.34 | 421 | 10 cm-esci-ammonia |
| Y-048 | 3.56 | 391 | 10 cm-esci-ammonia |
| Y-050 | 4.09 | 445 | 10 cm-esci-ammonia |
| Y-054 | 3.63 | 409 | 10 cm-esci-ammonia |
| Y-055 | 3.02 | 338 | 10 cm-esci-ammonia |
| Y-056 | 3.29 | 409 | 10 cm-esci-ammonia |
| Y-058 | 3.69 | 379 | 10 cm-esci-ammonia |
| Y-059 | 1.61 | 407 | 10 cm-apci-formic |
| Y-060 | 3.50 | 421 | 10 cm-apci-formic |
| Y-063 | 3.54 | 391 | 10 cm-esci-ammonia |
| Y-096 | 1.90 | 419 | 10 cm-apci-formic |

### General Synthesis Procedure E

Compounds were synthesised starting from 2-amino-5-bromopyridine or 2-amino-5-bromo-pyrazine following the scheme illustrated below.

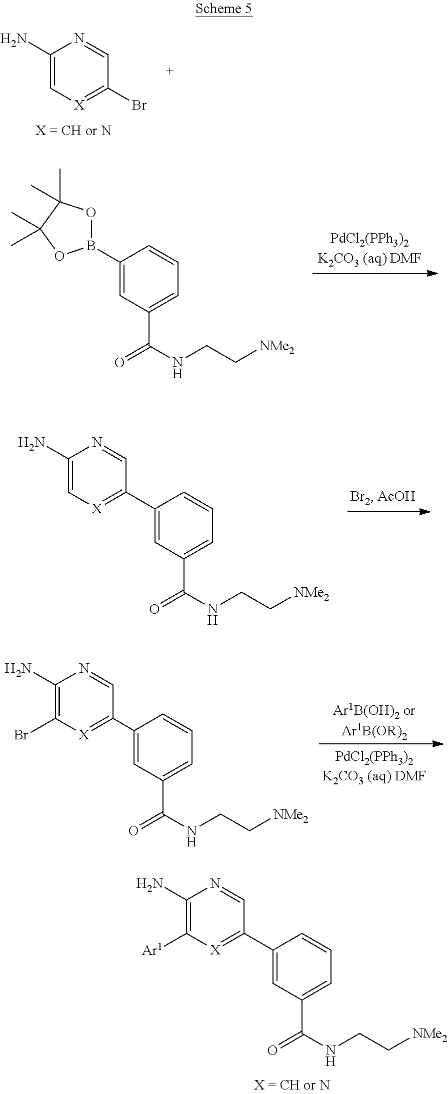

### Synthesis 12

### 3-(6-Amino-pyridin-3-yl)-N-(2-dimethylamino-ethyl)-benzamide

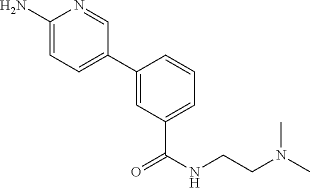

To a degassed suspension of 2-amino-5-bromopyridine (0.172 g; 1 mmol), and N-(2-dimethylamino-ethyl)-3-(4,4,5,5-tetramethyl-[1,3,2]dioxaborolan-2-yl)-benzamide (0.318 g; 1 mmol) in DMF (2 mL) and water (0.5 mL) was added potassium carbonate (414 mg; 3 mmol) andbis(triphenylphosphine)palladium (II) dichloride (32 mg; 5 μmol). The mixture was heated in a Smith Creator® microwave at 130° C. for 800 seconds. The reaction was diluted with ethyl acetate (15 mL) and washed with water (10 mL), brine (10 mL), and dried over MgSO₄, filtered, and evaporated. The crude product was chromatographed on silica eluted with 5% methanol (containing 0.1% ammonia) in dichloromethane. Appropriate fractions were pooled and evaporated to give the title compound as a cream coloured solid (170 mg). ¹H (400 MHz, CDCl₃) 8.36 (1H, s), 7.95 (1H, s), 7.64 (3H, m), 7.50 (1H, t), 6.80 (1H, bs), 6.59 (1H, d), 4.53 (2H, bs), 3.54 (2H, q), 2.53 (2H, t), 2.28 (6H, s). LCMS (method B) R_{T} 2.70, MI 285 (M+H⁺).

### Synthesis 13

### 3-(6-Amino-5-bromo-pyridin-3-yl)-N-(2-dimethylamino-ethyl)benzamide

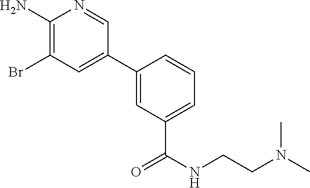

To a stirred solution of 3-(6-amino-pyridin-3-yl)-N-(2-dimethylamino-ethyl)-benzamide (0.284 g; 1 mmol) in glacial acetic acid (5 mL) at ambient temperature was added bromine (0.16 g; 1 mmol) dropwise and the mixture stirred for 2 hours. The solvent was removed under vacuum to give a brown gum. The crude product was treated with saturated sodium bicarbonate solution (10 mL) and the residue extracted with ethyl acatete (3×20 mL). The combined organics were dried over MgSO₄, filtered, and evaporated to give the title compound as a yellow solid (0.290 g). ¹H (400 MHz, CDCl₃) 8.30 (1H, s), 7.95 (2H, s), 7.69 (1H, d), 7.62 (1H, d), 7.50 (1H, t), 6.90 (1H, bs), 5.01 (2H, bs), 3.56 (2H, q), 2.56 (2H, t), 2.28 (6H, s). LCMS (method B) R_{T} 2.94, MI 363:365 (1:1 isotopes, M+H⁺).

### Synthesis 14

### 3-[6-Amino-5-(4-fluoro-3-trifluoromethyl-phenyl)-pyridin-3-yl]-N-(2-dimethylamino-ethyl)-benzamide

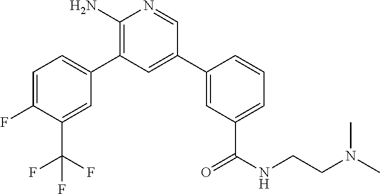

A degassed solution of 3-(6-amino-5-bromo-pyridin-3-yl)-N-(2-dimethylamino-ethyl)benzamide (40 mg; 0.11 mmol; 0.5 ml of a 0.22 M stock solution in DMF), potassium carbonate (0.5 ml of a 0.725 M stock solution in water), and bis(triphenylphosphine)palladium (II) dichloride (0.5 mL of a 0.011M stock solution in DMF) was added to 4-fluoro-3-trifluoromethylphenylboronic acid (23 mg; 0.11 mmol) and the mixture heated at 90° C. on a STEM block for 18 hours. The solvent was removed under vacuum (Genevac) and the residue treated with water (2 mL) and extracted with ethyl acetate (2 mL). The organic phase was separated and the organic phase evaporated (Genevac). The crude product was purified by Prep HPLC using method B to give the title compound as a cream coloured solid. ¹H (400 MHz, CDCl₃) 8.40 (1H, s), 7.99 (1H, s), 7.69 (5H, m), 7.65 (1H, t), 7.34 (1H, t), 6.86 (1H, bs), 4.61 (2H, bs), 3.55 (2H, q), 2.54 (2H, t), 2.28 (6H, s). LCMS (method B) R_{T} 4.28, MI 447 (M+H⁺).

The following compounds were synthesised in an analogous manner using General Synthesis Procedure E starting from 2-amino-5-bromopyridine.

Analytical LCMS

| ID No. | retention time (min) | MI + H | LCMS method |
|---|---|---|---|
| Y-005 | 1.65 | 392 | 10 cm-apci-formic |
| Y-006 | 3.44 | 396 | 10 cm-esci-ammonia |
| Y-007 | 2.38 | 378 | 10 cm-apci-formic |
| Y-008 | 1.63 | 392 | 10 cm-esp-formic |
| Y-011 | 4.22 | 417 | 10 cm-esci-ammonia |
| Y-012 | 4.06 | 401 | 10 cm-esci-ammonia |
| Y-013 | 4.54 | 437 | 10 cm-esci-ammonia |
| Y-014 | 4.76 | 437 | 10 cm-esci-ammonia |
| Y-015 | 4.81 | 467 | 10 cm-esci-ammonia |
| Y-018 | 3.44 | 412 | 10 cm-esci-ammonia |
| Y-020 | 4.38 | 411 | 10 cm-esci-ammonia |
| Y-024 | 3.62 | 367 | 10 cm-esci-ammonia |
| Y-027 | 3.73 | 414 | 10 cm-esci-ammonia |
| Y-028 | 3.81 | 403 | 10 cm-esci-ammonia |
| Y-029 | 3.06 | 423 | 10 cm-esci-ammonia |
| Y-030 | 4.14 | 467 | 10 cm-esci-ammonia |
| Y-031 | 4.98 | 455 | 10 cm-esci-ammonia |
| Y-032 | 4.14 | 453 | 10 cm-esci-ammonia |
| Y-033 | 3.60 | 397 | 10 cm-esci-ammonia |
| Y-036 | 4.03 | 389 | 10 cm-esci-ammonia |
| Y-037 | 4.06 | 429 | 10 cm-esci-ammonia |
| Y-038 | 4.63 | 467 | 10 cm-esci-ammonia |
| Y-039 | 3.76 | 413 | 10 cm-esci-ammonia |
| Y-040 | 3.74 | 395 | 10 cm-esci-ammonia |
| Y-041 | 3.54 | 386 | 10 cm-esci-ammonia |
| Y-042 | 2.27 | 395 | 10 cm-esci-ammonia |
| Y-043 | 3.84 | 409 | 10 cm-esci-ammonia |
| Y-044 | 4.06 | 393 | 10 cm-esci-ammonia |
| Y-045 | 4.60 | 455 | 10 cm-esci-ammonia |
| Y-046 | 4.52 | 419 | 10 cm-esci-ammonia |
| Y-047 | 3.62 | 405 | 10 cm-esci-ammonia |
| Y-049 | 2.02 | 453 | 10 cm-esp-formic |
| Y-051 | 4.66 | 467 | 10 cm-esci-ammonia |
| Y-052 | 4.17 | 413 | 10 cm-esci-ammonia |
| Y-053 | 4.73 | 421 | 10 cm-esci-ammonia |
| Y-057 | 4.27 | 447 | 10 cm-esci-ammonia |
| Y-061 | 2.87 | 391 | 10 cm-esci-ammonia |
| Y-062 | 5.79 | 417 | 10 cm-esci-ammonia |
| Y-064 | 4.00 | 445 | 10 cm-esci-ammonia |
| Y-065 | 4.03 | 461 | 10 cm-esci-ammonia |
| Y-066 | 3.19 | 449 | 10 cm-esci-ammonia |
| Y-067 | 4.01 | 453 | 10 cm-esci-ammonia |
| Y-068 | 4.60 | 417 | 10 cm-esci-ammonia |
| Y-069 | 3.95 | 429 | 10 cm-esci-ammonia |
| Y-070 | 3.98 | 465 | 10 cm-esci-ammonia |
| Y-072 | 2.58 | 439 | 10 cm-esci-bicarb |
| Y-073 | 1.90 | 405 | 10 cm-esci-bicarb |
| Y-076 | 2.34 | 418 | 10 cm-esci-bicarb |
| Y-077 | 2.43 | 377 | 10 cm-esci-bicarb |
| Y-078 | 2.50 | 412 | 10 cm-esci-bicarb |
| Y-079 | 1.68 | 377 | 10 cm-apci-formic |
| Y-080 | 2.63 | 400 | 10 cm-esci-bicarb |
| Y-081 | 1.89 | 421 | 10 cm-apci-formic |
| Y-082 | 1.81 | 427 | 10 cm-apci-formic |
| Y-083 | 1.87 | 392 | 10 cm-apci-formic |
| Y-084 | 1.43 | 362 | 10 cm-apci-formic |
| Y-085 | 1.77 | 396 | 10 cm-apci-formic |
| Y-087 | 2.08 | 441 | 10 cm-apci-formic |
| Y-088 | 1.82 | 451 | 10 cm-apci-formic |
| Y-089 | 1.65 | 392 | 10 cm-apci-formic |
| Y-090 | 1.83 | 412 | 10 cm-apci-formic |
| Y-091 | 1.69 | 413 | 10 cm-apci-formic |
| Y-094 | 2.45 | 401 | 10 cm-esci-bicarb |
| Y-095 | 3.68 | 455 | 10 cm-esci-bicarb |

The following compounds were synthesised in an analogous manner using General Synthesis Procedure E starting from 2-amino-5-bromopyrazine.

Analytical LCMS

| ID No. | retention time (min) | MI + H | LCMS method |
|---|---|---|---|
| X-023 | 2.86 | 428 | 10 cm-esci-bicarb |
| X-024 | 2.76 | 393 | 10 cm-esci-bicarb |
| X-025 | 3.48 | 442 | 10 cm-esci-bicarb |
| X-026 | 2.21 | 392 | 10 cm-apci-formic |

### General Synthesis Procedure F

Compounds were synthesised starting from the appropriate iodopyrimidine following the scheme illustrated below.

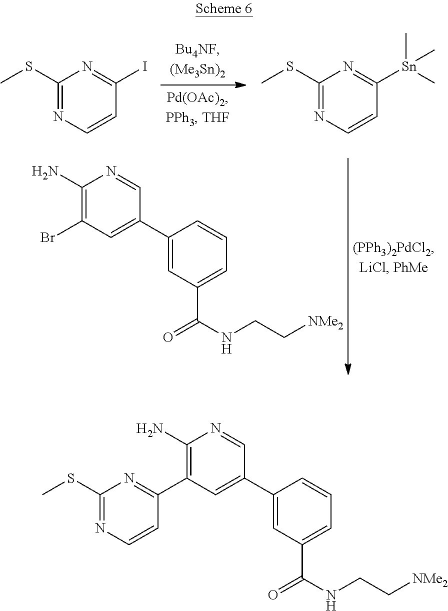

### Synthesis 15

### 2-Methylsulfanyl-4-trimethylstannylpyrimidine

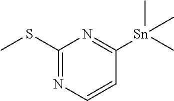

To a mixture of 2-methylsulfanyl-4-iodopyrimidine (0.40 g; 1.58 mmol), hexamethylditin (0.5 mL), palladium (II) acetate (23 mg; 0.10 mmol) and triphenylphosphine (45 mg; 0.16 mmol) in dry THF (10 mL) was added tetrabutylammonium fluoride (2.5 mL of a 1 M solution in THF). After the addition the mixture was left to stir at room temperature overnight. The solvent was removed by evaporation under reduced pressure to give a crude oil which was chromatographed on alumina eluted with 9:1 hexane/ethyl acetate to give the title compound as a colourless oil (0.26 g, 57%). ¹H (400 MHz, CDCl₃) 8.29 (1H, d), 7.12 (1H, d), 2.58 (3H, s), 0.36 (9H, s).

### Synthesis 16

### 3-[6-Amino-5-(2-methylsulfanyl-pyrimidin-4-yl)-pyridin-3-yl]-N-(2-dimethylamino-ethyl)-benzamide (Y-086)

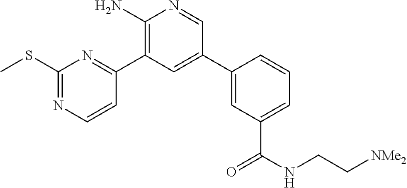

To a mixture of 2-methylthio-4-trimethylstannylpyrimidine (50 mg; 0.17 mmol), 3-(6-amino-5-bromo-pyridin-3-yl)-N-(2-dimethylamino-ethyl)benzamide (63 mg; 0.17 mmol), bis(triphenylphosphine)palladium (II) dichloride (12 mg; 0.017 mmol) and lithium chloride (30 mg; 0.7 mmol) was added toluene (3 mL, degassed). The mixture was heated in the Smith Creator® microwave at 100° C. for 15 minutes. The solvent was removed by evaporation under reduced pressure and the resultant residue was purified by preparative

HPLC to give the title compound as a yellow solid (3 mg). ¹H (400 MHz, CD₃OD) 8.64 (1H, d), 8.49 (2H, s), 8.14 (1H, s), 7.86 (2H, m), 7.79 (1H, d), 7.60 (1H, t), 3.65 (2H, t), 2.80 (2H, t), 2.65 (3H, s), 2.49 (6H, s). LCMS (method A) R_{T} 1.91, MI 409 (M+H⁺).

The following compound was synthesised in an analogous manner using General Synthesis Procedure F starting from 4-iodo-6-methoxypyrimidine:

Analytical LCMS

| ID No. | retention time (min) | MI + H | LCMS method |
|---|---|---|---|
| Y-093 | 3.00 | 393 | 10 cm-esci-bicarb |

### General Synthesis Procedure G

3-[6-amino-5-(3-methoxy-phenyl)-pyridin-3-yl]-benzoic acid was synthesised via General Synthesis Procedure D and then further manipulated following the scheme illustrated below.

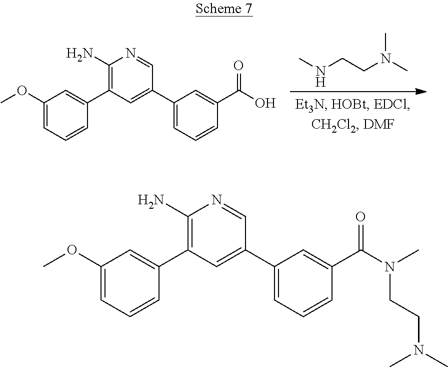

### Synthesis 17

### 3-[6-Amino-5-(3-methoxy-phenyl)-pyridin-3-yl]-N-(2-dimethylamino-ethyl)-N-methyl-benzamide (Y-075)

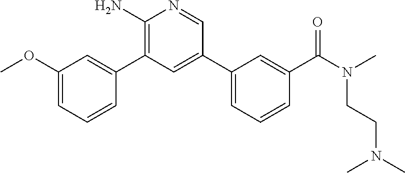

To a stirred solution of 3-[6-amino-5-(3-methoxy-phenyl)-pyridin-3-yl]-benzoic acid (48 mg; 0.15 mmol) in dichloromethane (1.5 mL) and N,N-dimethylformamide (1.5 mL) was added N,N,N′-trimethylethylenediamine (16 mg; 0.16 mmol), triethylamine (16 mg; 0.16 mmol), 1-hydroxybenzotriazole (21 mg; 0.16 mmol) and 1-(3-dimethylaminopropyl)carbodiimide hydrochloride (30 mg; 0.16 mmol). After 18 h at room temperature the reaction mixture was concentrated to dryness under reduced pressure. The crude product was chromatographed on silica eluted with dichloromethane/methanol/ammonia. Appropriate fractions were pooled and evaporated to give the title compound as a cream gum (25 mg). ¹H (400 MHz, CDCl₃) 8.42 (1H, s), 7.62 (1H, s), 7.57 (1H, s), 7.53 (1H, d), 7.43 (1H, d), 7.39 (1H, t), 7.31 (1H, d), 7.08 (1H, d), 7.02 (1H, s), 6.94 (1H, d), 4.71 (2H, s), 3.88 (3H, s), 3.69 (rotamer A, 2H, br. s), 3.38 (rotamer B, 2H, br. s), 3.11 (rotamer A, 3H, br. s), 3.01 (rotamer B, 3H, br. s), 2.60 (rotamer A, 2H, br. s), 2.40 (rotamer B, 2H, br. s), 2.30 (rotamer A, 6H, br. s), 2.06 (rotamer B, 6H, br. s). LCMS (method A) R_{T} 1.85, MI 405 (M+H⁺).

The following compound was synthesised in an analogous manner using General Synthesis Procedure G:

Analytical LCMS

| ID No. | retention time (min) | MI + H | LCMS method |
|---|---|---|---|
| Y-092 | 1.89 | 405 | 10 cm-apci-formic |

### Synthesis 18

### 3-(2-amino-2′-hydroxy-[3,4′]bipyridinyl-5-yl)-N-(2-dimethylamino-ethyl)-benzamide (Y-071)

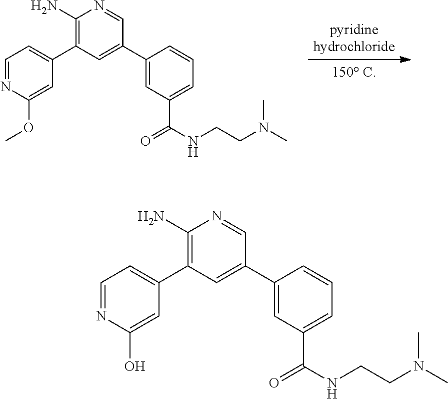

A mixture of 3-(2-amino-2′-methoxy-[3,4]′bipyridinyl-5-yl)-N-(2-dimethylamino-ethyl)-benzamide (Y-005) (80 mg; 0.20 mmol) and pyridine hydrochloride (116 mg; 1.02 mmol) was heated at 150° C. for 15 minutes. After cooling to room temperature, the crude product was chromatographed on silica eluted with dichloromethane/methanol/ammonia. Appropriate fractions were pooled and evaporated to give the title compound as a pale yellow solid (27 mg). ¹H (400 MHz, CD₃OD) 8.40 (1H, s), 8.10 (1H, s), 7.86 (1H, s), 7.82 (2H, t), 7.58 (1H, d), 7.56 (1H, m), 6.75 (1H, s), 6.64 (1H, d), 3.61 (2H, t), 2.70 (2H, t), 2.45 (6H, s) LCMS (method B) R_{T} 2.07, MI 378 (M+H⁺).

N-(2-dimethylamino-ethyl)-3-[5-(3-methoxy-phenyl)-6-methylamino-pyridin-3-yl]-benzamide (Y-074) was prepared, as described below, as shown in the following scheme.

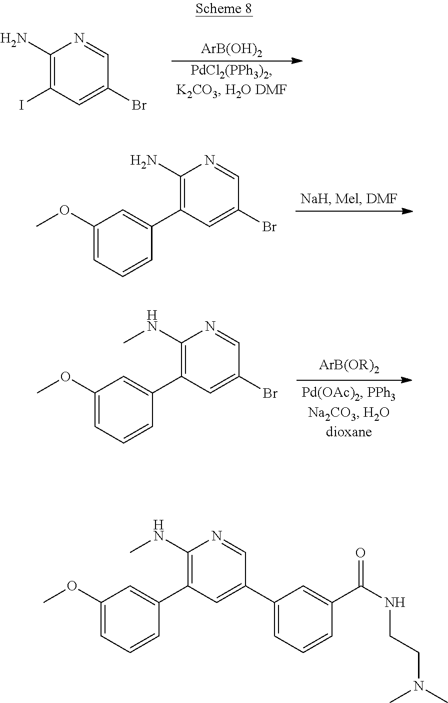

### N-(2-Dimethylamino-ethyl)-3-[5-(3-methoxy-phenyl)-6-methylamino-pyridin-3-yl]-benzamide (Y-074)

### Synthesis 19

### 5-Bromo-3-(3-methoxyphenyl)-pyridin-2-ylamine

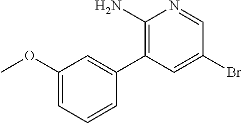

To a degassed mixture of 5-bromo-3-iodo-pyridin-2-ylamine (1.79 g; 6.0 mmol) and 3-methoxybenzeneboronic acid (0.92 g; 6.0 mmol) in N,N-dimethylformamide (18 mL) and aqueous potassium carbonate (9 mL; 2.0 M solution) was added bis(triphenylphosphine) palladium(II) chloride (210 mg; 0.30 mmol) and the mixture was heated at 85° C. for 18 hours. The reaction mixture was partitioned between ethyl acetate (50 mL) and water (25 mL). The organic phase was separated, dried over MgSO₄, filtered, and evaporated to dryness. The crude product was chromatographed on silica eluted with petroleum ether/ethyl acetate. Appropriate fractions were pooled and evaporated to give the title compound as a golden brown solid (0.68 g). ¹H (400 MHz, d₆-DMSO) 8.03 (1H, s), 7.52 (1H, s), 7.37 (1H, t), 7.01 (3H, m), 5.88 (2H, s), 3.86 (3H, s). LCMS (method B) R_{T}=3.46, MI 279:281 (1:1 isotopes M+H⁺).

### Synthesis 20

### [5-Bromo-3-(3-methoxyphenyl)-pyridin-2-yl]methylamine

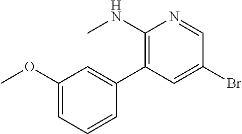

To a stirred solution of 5-bromo-3-(3-methoxyphenyl)-pyridin-2-ylamine (47 mg; 0.17 mmol) in N,N-dimethylformamide (2 mL) under a nitrogen atmosphere was added sodium hydride (60% dispersion in mineral oil; 7.5 mg; 0.19 mmol). After 10 minutes, iodomethane (26.5 mg; 0.19 mmol) was added and the reaction mixture was stirred for 2 hours. The solution was concentrated to dryness under reduced pressure then partitioned between dichloromethane (30 mL) and water (15 mL). The organic phase was separated, dried over MgSO₄, filtered, and evaporated to dryness. The crude product was chromatographed on silica eluted with petroleum ether/ethyl acetate. Appropriate fractions were pooled and evaporated to give the title compound as a white solid (27 mg). ¹H (400 MHz, CDCl₃) 8.17 (1H, s), 7.34 (2H, m), 6.93 (2H, m), 6.89 (1H, s), 4.66 (1H, s), 3.85 (3H, s), 2.91 (3H, d). LCMS (method B) R_{T}=3.84, MI 293:295 (1:1 isotopes M+H⁺).

### Synthesis 21

### N-(2-Dimethylamino-ethyl)-3-[5-(3-methoxy-phenyl)-6-methylamino-pyridin-3-yl]-benzamide (Y-074)

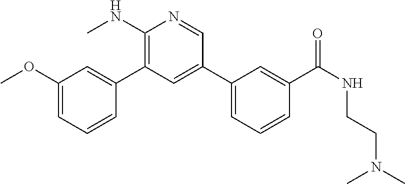

To a solution of [5-bromo-3-(3-methoxyphenyl)-pyridin-2-yl]methylamine (27 mg; 0.092 mmol) and N-(2-dimethylamino-ethyl)-3-(4,4,5,5-tetramethyl-[1,3,2]dioxaborolan-2-yl)-benzamide (25 mg; 0.101 mmol) in dioxane (0.5 mL) was added palladium (II) acetate (1 mg; 0.0046 mmol) and triphenylphosphine (5 mg; 0.0184 mmol). Aqueous sodium carbonate (0.25 mL; 1.5 M solution) was added and the mixture heated in a Smith Creator® microwave at 130° C. for 800 seconds. The reaction was diluted with dichloromethane (6 mL) and water (2 mL) and poured through a PTFE separation frit. The dichloromethane filtrate was collected and evaporated. The crude product was chromatographed on silica eluted with dichloromethane/methanol/ammonia. Appropriate fractions were pooled and evaporated to give a solid which was further purified by preparative HPLC (Method A) to give the title compound (formate salt) as a white solid (3.4 mg). ¹H (400 MHz, CDCl₃) 8.61 (1H, bs), 8.46 (1H, s), 8.29 (1H, s), 8.10 (1H, s), 7.81 (1H, d), 7.68 (1H, d), 7.61 (1H, s), 7.47 (1H, t), 7.39 (1H, t), 7.02 (1H, d), 6.98 (1H, s), 6.93 (1H, m), 4.72 (1H, bs) 3.85 (5H, m), 3.18 (2H, m), 3.01 (3H, s), 2.77 (6H, s). LCMS (method A) R_{T} 1.92, MI 405 (M+H⁺).

### Biological Methods

### Expression and Purification of PKD1 Protein

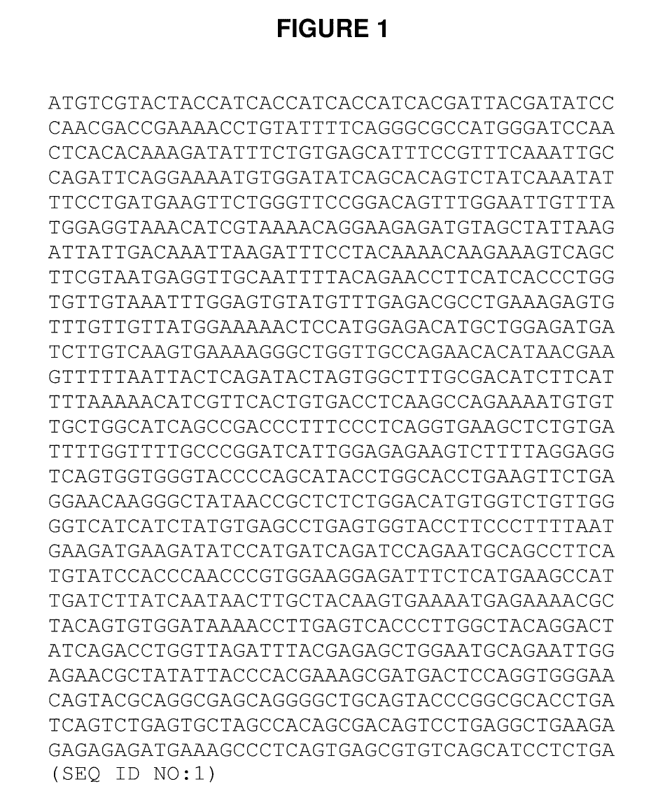

The DNA sequence corresponding to murine PKD1 (see FIG. 1) was inserted into pFastBAc Htb (Invitrogen, USA) at BamH1 and EcoR1 sites using standard molecular biology techniques.

The PKD1 described above was expressed as a hexahistidine tagged protein construct using a commercially available baculoviral expression system that induces protein production in insect cell culture (Bac-to-Bac® HT Baculovirus Expression System, Invitrogen). Protein was typically expressed by inoculating 1 L of sf9 cells with a genetically modified baculovirus containing the gene for the kinase domain of PKD1. Sf9 cells were obtained from ICR Ltd.

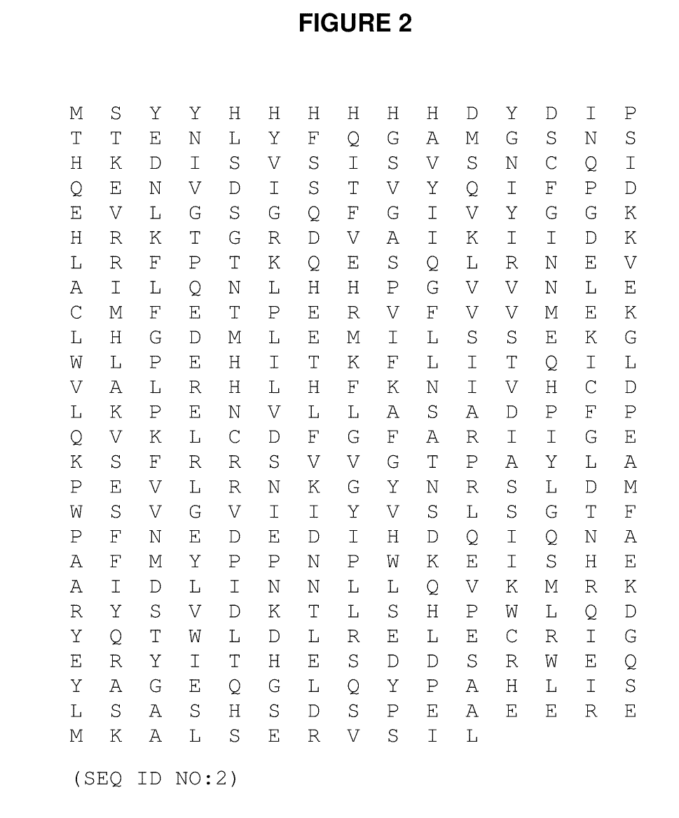

Purification of PKD1 was achieved by standard chromatographic procedures. Capture from crude centrifuged lysed cell supernatant was achieved using metal affinity chromatography (GE Healthcare Life Sciences, HiTrap Chelating chromatography column), and fractions showing PKD1 (as assessed by gel electrophoresis and western blot) were further purified by a single polish purification step performed using a mono Q anion exchange chromatography system (GE Healthcare Life Sciences, HiTrap HP Q column). Purified PKD1 (the amino acid sequence is shown in FIG. 2) was tested for activity in a commercially available kinase assay (Molecular Devices IMAP kinase assay kit; see, e.g., Singh et al., 2005). This protocol describes the method for screening compounds as inhibitors of Protein Kinase D activity in a 384 well microplate format fluorescence polarisation IMAP assay performed using the Biomek FX.

Compounds were screened for inhibition of Protein Kinase D activity at 30 μM in a 384 well microplate format using the fluorescence polarisation based IMAP assay (Molecular Devices Inc. USA.).

### PKD1 Murine Kinase Domain Enzyme Activity Assay

### Reagents

Kinase Assay Reaction Buffer: This consisted of 0.22 μM filtered 25 mM HEPES and 2 mM MgCl₂ pH 7.5.

Kinase Enzyme: Murine PKD1 kinase domain at ˜100 μg/mL, was purified from baculovirus (as described above), obtained from aliquots stored at −70° C. PKD was prepared with a final concentration of 0.1 μg/mL by diluting 1:300 in Reaction Buffer (30 μL per 9 mL-5 mL per plate with an additional 4 mL dead volume) and vortexing prior to use. It was necessary to check this concentration regularly in case of enzyme degradation.

Substrate: Fluorescein labelled glycogen synthase-derived peptide (FI)-KKLNRTLSVA (also known as MAPKAP K2 substrate) was obtained from Molecular Devices (Product code R7127). It was used at 300 nM by diluting 20 μM stock 1:66 in Kinase/Reaction Buffer (135 μL per 9 mL; 75 μL per 5 mL Reaction Buffer for blank wells requiring <1 mL per place with an additional 4 mL dead volume).

ATP: (Obtained from Sigma, product code A-7699). A 1 mM ATP stock in Reaction Buffer was prepared from a 10 mM stock in 20 mM NaOH and stored as aliquots at −70° C. It was used at 40 μM by diluting 1 mM stock 1:25 in Reaction Buffer (240 μL per 6 mL-2 mL per plate with an additional 4 mL dead volume) and vortexing prior to use.

IMAP Reagents: IMAP Binding Reagent (product code R7207) and Binding Buffer (product code R7208) were obtained from Molecular Devices and stored at +4° C. The beads were gently re-suspended before diluting by 1:400 in buffer (Binding Buffer is supplied as a 5× stock and so was diluted with water prior to use) and then vortexing before addition to wells. 16 mL water with 4 mL Binding Buffer and 50 μL Binding Reagent were used per plate (17 mL per plate with an additional 3 mL dead volume).

### Method

13 μL Kinase/Substrate in Reaction Buffer was added to ‘test’ and all ‘control’ wells of a Corning black low binding 384 well (90 μL volume) microplate to give 0.2 μg/mL and 200 nM reaction concentration respectively. 13 μL Substrate in Reaction Buffer was added to ‘blank’ wells to give 200 nM reaction concentration. 2 μL test compound in 10% DMSO/water was added to ‘test’ wells to give final concentrations ranging from 100 to 0.001 μM. 2 μL 10% DMSO/water was added to ‘blank’ and ‘control’ wells. 5 μL ATP in Reaction Buffer was added to all wells to give 10 μM reaction concentration. The reaction mixture was then incubated at room temperature for 60 minutes. The incubation period was followed by the addition of 40 μL IMAP Binding Reagent in Binding Buffer to all wells. The reaction was then further incubated at room temperature for minutes. The fluorescence polarisation of the substrate in each well was recorded using an analyst microplate reader (Molecular Devices) with asingle read at Ex485 Em535 (Analyst settings: Z Height 5 mm, G Factor 0.95, Reads/well 1, Integration 100000 μs, Gain Sensitivity 2).

Percentage inhibition was calculated based on activity of the test sample minus the average values in the blank wells relative to the average values measured in control wells minus the average values in the blank wells.

IC₅₀ values were calculated from 10 point dose sigmoid ‘dose-response’ curves using Xlfit software (IDBS inc, USA). Data were fitted to a 4 parameter logistic model/sigmoidal dose response:

$$Fit = A + ( \frac{( B - A )}{( 1 + {( \frac{C}{X} )}^{D} )} )$$

where:
A=fit minimum (locked to 0);
B=fit maximum (locked to 100);
C=fit midpoint (pre-fit to 1);
D=slope at linear portion of curve, hillslope (pre-fit to 0.1)

The value for C represents the IC₅₀ of the test compound

### PKD1 (Human Full Length) Enzyme Activity Assay

Kinase Assay Reaction Buffer: This consisted of 0.22 μM filtered 25 mM HEPES and 2 mM MgCl₂ pH 7.5.

Kinase Enzyme: Human full length PKD1 at ˜100 μg/mL purchased from Upstate Ltd (Product code 14-508) was obtained from aliquots stored at −70° C. It was prepared with a final concentration of 0.3 μg/mL by diluting 1:300 in Reaction Buffer (30 μL per 9 mL-5 mL per plate with an additional 4 mL dead volume) and vortexing prior to use.

Substrate: Fluorescein labelled glycogen synthase-derived peptide (FI)-KKLNRTLSVA (also known as MAPKAP K2 substrate) was obtained from Molecular Devices (Product code R7127). It was used at 200 nM by diluting 20 μM stock 1:66 in Kinase/Reaction Buffer (135 μL per 9 mL; 75 μL per 5 mL Reaction Buffer for blank wells requiring <1 mL per place with an additional 4 mL dead volume).

ATP: (Obtained from Sigma, product code A-7699). A 1 mM ATP stock in Reaction Buffer was prepared from a 10 mM stock in 20 mM NaOH and stored as aliquots at −70° C. It was used at 40 μM by diluting 1 mM stock 1:25 in Reaction Buffer (240 μL per 6 mL-2 mL per plate with an additional 4 mL dead volume) and vortexing prior to use.

IMAP Reagents: IMAP Binding Reagent (product code R7207) and Binding Buffer (product code R7208) were obtained from Molecular Devices, and stored at +4° C. The beads were gently re-suspended before diluting by 1:400 in buffer (Binding Buffer was supplied as a 5× stock and so was diluted with water prior to use) and then vortexing before addition to wells. 16 mL water with 4 mL Binding Buffer and 50 μL Binding Reagent were used per plate (17 mL per plate with an additional 3 mL dead volume).

### Method

13 μL Kinase/Substrate in Reaction Buffer was added to ‘test’ and all ‘control’ wells of a Corning black low binding 384 well (90 μL volume) microplate to give 0.2 μg/mL and 200 nM reaction concentration respectively. 13 μL Substrate in Reaction Buffer was added to ‘blank’ wells to give 200 nM reaction concentration. 2 μL test compound in 10% DMSO/water was added to ‘test’ wells to give final concentrations ranging from 100 to 0.001 μM. 2 μL 10% DMSO/water was added to ‘blank’ and ‘control’ wells. 5 μL ATP in Reaction Buffer was added to all wells to give 10 μM reaction concentration. The reaction mixture was then incubated at room temperature for 25 minutes. The incubation period was followed by the addition of 40 μL IMAP Binding Reagent in Binding Buffer to all wells. The reaction was then further incubated at room temperature for ≧30 minutes.

The fluorescence polarisation of the substrate in each well was recorded using an analyst microplate reader (Molecular Devices) with a single read at Ex485 Em535 (Analyst settings: Z Height 5 mm, G Factor 0.95, Reads/well 1, Integration 100000 μs, Gain Sensitivity 2).

Percentage inhibition was calculated based on activity of the test sample minus the average values in the blank wells relative to the average values measured in control wells minus the average values in the blank wells.

IC₅₀ values were calculated from 10 point dose sigmoid ‘dose-response’ curves using Xlfit software (IDBS inc, USA). Data were fitted to a 4 parameter logistic model/sigmoidal dose response:

$$Fit = A + ( \frac{( B - A )}{( 1 + {( \frac{C}{X} )}^{D} )} )$$

where:
A=fit minimum (locked to 0);
B=fit maximum (locked to 100);
C=fit midpoint (pre-fit to 1);
D=slope at linear portion of curve, hillslope (pre-fit to 0.1)

The value for C represents the IC₅₀ of the test compound

### PKD2 (Human Full Length) Enzyme Activity Assay

### Reagents

Kinase Assay Reaction Buffer: This consisted of 0.22 μM filtered 25 mM HEPES and 10 mM MgCl₂ pH 7.5.

Kinase: human full length PKD2 at ˜100 μg/mL was purchased from Upstate Ltd (Product code 14-506), and obtained from aliquots stored at −70° C. It was prepared with a final concentration of 0.1 μg/mL by diluting 1:300 in Reaction Buffer (30 μL per 9 mL-5 mL per plate with an additional 4 mL dead volume) and vortexing prior to use. It is necessary to check this concentration regularly in case of enzyme degradation.

Substrate: Fluorescein labelled glycogen synthase-derived peptide (FI)-KKLNRTLSVA (also known as MAPKAP K2 substrate) was obtained from Molecular Devices (Product code R7127). It was used at 2 μM by diluting 20 μM stock 1:10 in Kinase/Reaction Buffer (900 μL per 9 mL; 500 μL per 5 mL Reaction Buffer for blank wells requiring <1 mL per place with an additional 4 mL dead volume).

ATP: (Obtained from Sigma, product code A-7699). A 1 mM ATP stock in Reaction Buffer was prepared from a 10 mM stock in 20 mM NaOH and stored as aliquots at −70° C. It was used at 600 μM by diluting 100 mM stock 1:166.6 in Reaction Buffer (36 μL per 6 mL-2 mL per plate with an additional 4 mL dead volume) and vortexing prior to use.

IMAP Reagents: IMAP Binding Reagent (product code R7207) and Binding Buffer (product code R7208) were obtained from Molecular Devices. Both were stored at +4° C. The beads were gently resuspended before diluting by 1:400 in buffer (Binding Buffer is supplied as a 5× stock and so is diluted with water prior to use) and then vortexing before addition to wells. 16 mL water with 4 mL Binding Buffer and 50 μL Binding Reagent were used per plate (17 mL per plate with an additional 3 mL dead volume).

### Method

5 μL Kinase/Substrate in Reaction Buffer was added to ‘test’ and all ‘control’ wells of a Corning black low binding 384 well (90 μL volume) microplate to give 0.1 μg/mL and 2 μM reaction concentration respectively. 5 μL Substrate in Reaction Buffer was added to ‘blank’ wells to give 2 μM reaction concentration. 1 μL test compounds in 40% DMSO/water was added to ‘test’ wells to give final concentrations ranging from 100 to 0.001 μM. 1 μL 10% DMSO/water was added to ‘blank’ and ‘control’ wells. 4 μL ATP in Reaction Buffer was added to all wells to give 10 μM reaction concentration. The reaction mixture was then incubated at room temperature for 90 minutes. The incubation period was followed by the addition of 90 μL of cold 1× Reaction Buffer. 20 μL of the resulting solution was subsequently transferred to a fresh identical microplate. 40 μL IMAP Binding Reagent in Binding Buffer was added to all wells of this new microplate. The reaction was further incubated at room temperature for minutes. The fluorescence polarisation of the peptide substrate was measured using an analyst (Molecular devices) microplate reader with a single read at Ex485 Em535 (Analyst settings: Z Height 5 mm, G Factor 0.95, Reads/well 1, Integration 100000 μs, Gain Sensitivity 2).

Percentage inhibition was calculated based on activity of the test sample minus the average values in the blank wells relative to the average values measured in control wells minus the average values in the blank wells.

IC₅₀ values were calculated from 10 point dose sigmoid ‘dose-response’ curves using Xlfit software (IDBS inc, USA). Data were fitted to a 4 parameter logistic model/sigmoidal dose response:

$$Fit = A + ( \frac{( B - A )}{( 1 + {( \frac{C}{X} )}^{D} )} )$$

where:
A=fit minimum (locked to 0);
B=fit maximum (locked to 100);
C=fit midpoint (pre-fit to 1);
D=slope at linear portion of curve, hillslope (pre-fit to 0.1)

The value for C represents the IC₅₀ of the test compound.

### Western Blot 916 Assay

PANC-1 (ATCC CRL-1469) cells were seeded in 6 well plates. After overnight serum starvation of cells, cells were washed twice in 1 mL serum-free media per well, then treatments were added in serum-free media.

Cells were treated with 1 μM, 10 μM, or 30 μM of a pyridine benzamide compound (or with 3 μM GF1, for comparison purposes) for 1 hour. Then, 200 nM PDBu (phorbol, 12,13-dibutyrate) was added to the wells for 10 minutes. Two wells were used for each treatment.

Cells were then scraped into lysis buffer (40 μL per well), samples were homogenised, and protein concentration determined. Equal amounts of protein lysate (26 μg) were loaded onto on pre-cast gels (10%) for western analysis using an anti-PKD1 (human) Antibody (Cell Signaling Technology, No. 2052, Lot 3) and anti-phospho-PKD1 (human) (Ser916) Antibody (Cell Signaling Technology, No. 2051, Lot 3)

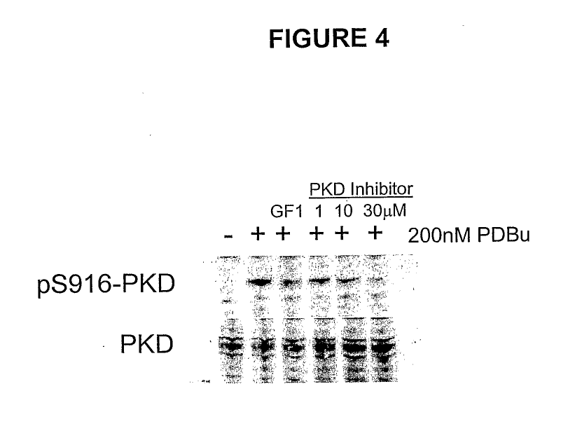

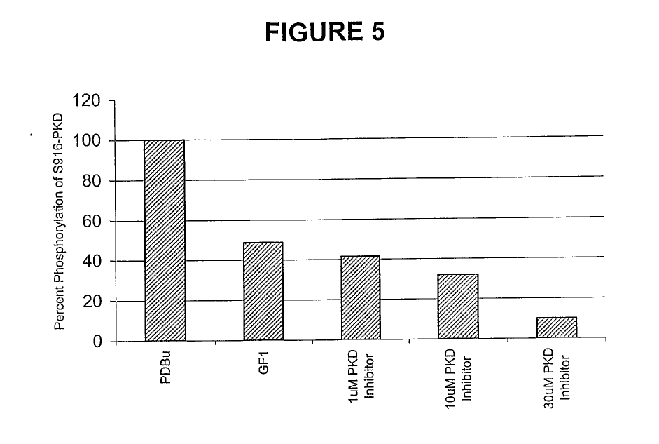

The results are shown in FIG. 4 and FIG. 5.

FIG. 4 is a photographic depiction of the western blot analysis of cell lysates of PANC-1 cells which were treated with increasing amounts (1, 10, 30 μM) of a pyridine benzamide compound. Cell lysates were analysed using an anti-PKD1 Antibody (lower panel) and anti-phospho-PKD1 (Ser916) Antibody (top panel).

FIG. 5 is a depiction of the quantification of the western blot as shown in FIG. 4. The shown columns represent the % phosphorylation as measured by densitometry of phosphor-PKD1 (Ser916) levels. The results were normalised to the measured PKD1 levels and expressed as % of the level of phosphorylation in the PDBu-stimulated control.

Both figures show that the pyridine benzamide compound inhibited PDBu-stimulated PKD1 Ser916 phosphorylation in PANC-1 cells in a dose-responsive fashion with an IC₅₀ of approximately 4 μM.

### MTT Assay

This assay determines the cell toxicity of compounds using the MTT (3-[4,5-dimethylthiazol-2-yl]-2,5-diphenyltetrazolium bromide) assay. This assay can be used to assess the cytotoxicity, cell viability, and proliferation of cells. In living cells, the tetrazolium salt (MTT) is reduced to a coloured formazan product (1-[4,5-dimethylthiazol-2-yl]3,5-diphenylformazan), which can be quantified. The reduction of MTT is attributed to the mitochondrial function of cells.

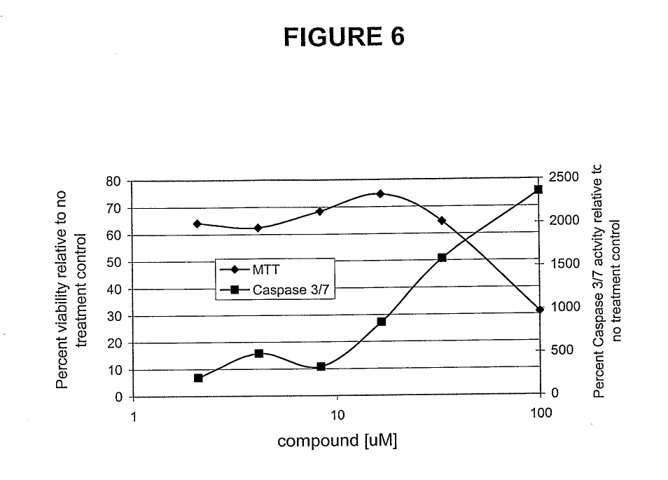

The cells were pelleted for 5 minutes at 1500 rpm (at 4° C.) and re-suspended in a small volume of media (E4+10% FCS; ˜3 mL/175 cm² flask). The cells were counted with a haemocytometer and diluted to a concentration of 1×10⁵ cells/mL. An aliquot of 100 μL per well across the 96 well plate was added from column 1 to 10, adding 100 μL media (no cells) to column 11, and 100 μL media and cells to column 12. The 96-well plate was placed in an incubator at 37° C., 5% CO₂ overnight. Cells were then serum starved for 16 hours (E4+0.5% FCS). A serial dilution of a pyridine benzamide or pyrazine benzamide compound (in E4+0.5% FCS) was carried out in a 96-well compound plate from column 1 to 10 ensuring good mixing in each well. The 96 well plates containing cells to be tested were removed from the incubator. 100 μL of compound/media solution was added and the plate was placed in the incubator at 37° C. 5% CO₂ for 1 hour. Cells were then treated with Neurotensin (NT; 50 nM) in the presence of compound for a further 47 hours (in E4+0.5% FCS; 37° C., 5% CO₂). At the end of the incubation, the media was aspirated off. 50 μL per well of 2 mg/mL MTT solution was added and the plates were placed back into the incubator for 2.5 to 4 hours. After incubation, the plates were removed from the incubator and the MTT solution was completely aspirated off the cells. 50 μL DMSO was added to each well and the plates agitated vigorously for 1 minute without introducing bubbles to the wells. The plates were read in a 96 well plate reader at 562 nm (Lab Systems, Ascent Multiscan). The results are shown in FIG. 6.

### Apoptosis Assay

PKD2 has been shown to play a role in cell survival through increasing cellular resistance to apoptosis (see, e.g., Trauzold et al., 2003; Storz et al., 2005). In addition, results from an siRNA screen of human kinases has identified PKD2 as a survival kinase (Mackeigan et al., 2005).

PANC-1 cells were seeded into 96 well plates (1×10⁴ cells/well in E4+10% FCS). Cells were serum starved (E4+0.5% FCS) for 16 hours and then treated with a pyridine benzamide or pyrazine benzamide compound for 1 hour prior to treatment with Neurotensin (NT; 50 nM) for a further 47 hours (in E4+0.5% FCS; total exposure to test compound was 48 hours). Cells were then assayed for Caspase3/7 activity (Caspase-Glo; Promega) according to the manufacturer's instructions.

The caspase assay was a homogenous luminescent assay that measures caspase 3 and 7 activities. The assay used here provided luminegenic caspase 3 and 7 substrate, which contained the tetrapeptide DEVD in a reagent optimised by the manufacturer for caspase activity, luciferase activity, and cell lysis. When added to the cell samples, these reagents resulted in cell lysis, followed by caspase cleavage of the substrate and generation of a luminescent signal produced by luciferase, whereby the luminescence was proportional to the amount of caspase activity present. An increase of caspase activity was proportional to increased apoptosis. The results are shown in FIG. 6.

Treatment of PANC-1 cells with increasing concentrations of pyridine benzamide or pyrazine benzamide compound (2-100 μM) for 48 hours resulted in a marked increase in caspase 3/7 activity (>23-fold increase at 100 μM) and a corresponding decrease in cell viability (70% decrease at 100 μM). These data suggest that tested compounds induced cell death by apoptosis (see FIG. 6).

FIG. 6 shows a graphic representation of the results obtained in the MTT and Caspase 3/7 assays. The depicted lines show the change in viability or induction of apoptosis in the presence of a pyridine benzamide compound. Cell viability was measured by the MTT assay and induction of apoptosis was measured by the caspase assay at 48 hours. The data are expressed as a % of the level in the corresponding control.

### Additional Biological Data

Biological data were obtained using the PKD1 (Murine Kinase Domain) Enzyme Activity Assay described above for the following 88 compounds: X-001 through X-022 and Y-001 through Y-066.

For these compounds, for the PKD1 (Murine Kinase Domain) Enzyme Activity Assay, the IC50 (μM) values are as follows:

at least 5 of the compounds tested have an IC50 of 0.01 μM or less;
at least 23 of the compounds tested have an IC50 of 0.1 μM or less;
at least 64 of the compounds tested have an IC50 of 1 μM or less;
at least 82 of the compounds tested have an IC50 of 10 μM or less.

Biological data were obtained using the PKD1 (Murine Kinase Domain) Enzyme Activity Assay described above for the following compounds: X-001 through X-027 and Y-001 through Y-096.

All of these compounds have an IC50 of less than 20 μM.

The following compounds have an IC50 of 1 μM or more, and less than 10 μM: X-001, X-002, X-003, X-010, X-015, X-018, Y-002, Y-003, Y-004, Y-006, Y-008, Y-018, Y-021, Y-030, Y-034, Y-046, Y-047, Y-054, Y-072, Y-073.

The following compounds have an IC50 of less than 1 μM: X-004, X-005, X-006, X-007, X-008, X-012, X-013, X-016, X-017, X-019, X-021, X-022, X-023, X-024, X-025, X-026, X-027, Y-001, Y-005, Y-007, Y-010, Y-011, Y-012, Y-013, Y-014, Y-015, Y-016, Y-017, Y-019, Y-020, Y-022, Y-023, Y-024, Y-025, Y-026, Y-027, Y-028, Y-029, Y-031, Y-033, Y-035, Y-036, Y-037, Y-038, Y-039, Y-040, Y-041, Y-042, Y-043, Y-044, Y-045, Y-048, Y-049, Y-050, Y-051, Y-052, Y-053, Y-055, Y-056, Y-057, Y-058, Y-059, Y-060, Y-061, Y-062, Y-063, Y-064, Y-065, Y-066, Y-067, Y-068, Y-069, Y-070, Y-071, Y-074, Y-075, Y-076, Y-077, Y-078, Y-079, Y-080, Y-081, Y-082, Y-083, Y-084, Y-085, Y-086, Y-087, Y-088, Y-089, Y-090, Y-091, Y-092, Y-093, Y-094, Y-095, Y-096.

For the PKD1 (Murine Kinase Domain) Enzyme Activity Assay, compound Y-059 has an IC50 (μM) value of 0.0085 μM.

For the PKD1 (Murine Kinase Domain) Enzyme Activity Assay, compound X-017 has an IC50 (μM) value of 0.012 μM.

Biological data were obtained using the PKD1 (Human Full Length) Enzyme Activity Assay described above for the following seven compounds: X-017, X-022, Y-004, Y-005, Y-026, Y-056, and Y-059.

For the PKD1 (Human Full Length) Enzyme Activity Assay, the IC50 (μM) values are as follows:

at least 3 of the compounds tested have an IC50 of 0.01 μM or less;
at least 5 of the compounds tested have an IC50 of 0.1 μM or less;
all of the compounds tested have an IC50 of 1 μM or less

For the PKD1 (Human Full Length) Enzyme Activity Assay, compound Y-059 has an IC50 (μM) value of 0.006 μM.

For the PKD1 (Human Full Length) Enzyme Activity Assay, compound X-017 has an IC50 (μM) value of 0.004 μM.

Biological data were obtained using the PKD2 (Human Full Length) Enzyme Activity Assay described above for the following five compounds: X-007, Y-004, Y-005, Y-056, and Y-059.

For the PKD2 (Human Full Length) Enzyme Activity Assay, the IC50 (μM) values are as follows:

at least 2 of the compounds had have an IC50 of 0.1 μM or less;
all of the compounds had have an IC50 of 1 μM or less.

For the PKD1 (Human Full Length) Enzyme Activity Assay, compound Y-059 has an IC50 (μM) value of 0.032 μM.

For the PKD1 (Human Full Length) Enzyme Activity Assay, compound X-007 has an IC50 (μM) value of 0.42 μM.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of patents and publications are cited above in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Bollag W B, Dodd M E, Shapiro B A. (2004). Protein kinase D and keratinocyte proliferation. Drug News Perspect. March; 17(2):117-26.
Bowden, E. T., Barth, M., Thomas, D., Glazer, R. I. & Mueller, S. C. (1999). An invasion-related complex of cortactin, paxillin and PKCμ associates with invadopodia at sites of extracellular matrix degradation. Oncogene 18(31), 4440-4449.
Doppler H, Storz P, Li J, Comb M J, Toker A., 2005, A phosphorylation state-specific antibody recognizes Hsp27, a novel substrate of protein kinase D. J Biol. Chem. 280(15)15013-15019.
Gamido C, Schmitt E, Cande C, Vahsen N, Parcellier A, Kroemer G. N (2003). HSP27 and HSP70: potentially oncogenic apoptosis inhibitors. Cell Cycle. 6, 579-584.
Guha S, Lunn J A, Santiskulvong C and Rozengurt, E (2003). Neurotensin stimulates PKC-dependent mitogenic signaling in human pancreatic carcinoma cell line PANC-1. Cancer Research 63, 2379-2387.
Guha, S, Rey, O and Rozengurt, E (2002). Neurotensin induces Protein Kinase C-dependent Protein Kinase D activation and DNA synthesis in human pancreatic carcinoma cell line, PANC-1. Cancer Research 62, 1632-1640.
Hurd C, Rozengurt E. (2003) Uncoupling of protein kinase D from suppression of EGF-dependent c-Jun phosphorylation in cancer cells. Biochem Biophys Res Commun. 302, 800-804.
Hurd C, Waldron R T, Rozengurt E. (2002). Protein kinase D complexes with C-Jun N-terminal kinase via activation loop phosphorylation and phosphorylates the C-Jun N-terminus. Oncogene. 21, 2154-2160.
Huynh Q K, McKinsey T A. (2006) Protein kinase D directly phosphorylates histone deacetylase 5 via a random sequential kinetic mechanism. Arch Biochem Biophys. 450, 141-148.
Johannes, F. J., Horn, J., Link, G., Haas, E., Siemienski, K., Wajant, H. & Pfizenmaier, K. (1998). Protein kinase Cmu downregulation of tumor-necrosis-factor-induced apoptosis correlates with enhanced expression of nuclear-factor-kappaB-dependent protective genes. Eur J Biochem. 257(1), 47-54.
Kennett, S. B., Roberts, J. D. & Olden, K. (2004). Requirement of PKCmu activation and calpain-mediated proteolysis for arachidonic acid-stimulated adhesion of MDA-MB-435 human mammary carcinoma cells to collagen type IV. J Biol Chem., 279, 3300-3307.
MacKeigan J P, Murphy L O, Blenis J. (2005), Sensitized RNAi screen of human kinases and phosphatases identifies new regulators of apoptosis and chemoresistance. Nat Cell Biol. 2005 June; 7(6):591-600.
Matthews, S. Rozengurt, E. & Cantrell, D. (2000b). Protein Kinase D: a selective target for antigen receptors and a downstream target for Protein Kinase C in lymphocytes. J Exp. Med. 191, 2075-82.
Matthews, S., Iglesias, T., Rozengurt, E. & Cantrell, D. (2000a). Spatial and temporal regulation of Protein Kinase D (PKD). EMBO J. 19, 2935-45.
McKinsey, T A and Olson, E N. (2005). Toward transcriptional therapies for the failing heart: chemical screens to modulate genes. J. Clin. Invest. 115:538-546.
Mihailovic T, Marx M, Auer A, Van Lint J, Schmid M, Weber, C and Seufferlein T (2004). Protein kinase D2 mediates activation of NfkappaB by Bcr-Abl in Bcr-Abl+ human myeloid leukemia cells. Cancer Research 64, 8939-8944.
Paolucci, L and Rozengurt, E (1999). Protein Kinase D in small cell lung cancer cells: Rapid activation through Protein Kinase C. Cancer Research 59, 572-577.
Qin L, Zeng H, Zhao D. 2006. Requirement of protein kinase D tyrosine phosphorylation for VEGF-A165-induced angiogenesis through its interaction and regulation of phospholipase Cgamma phosphorylation. J. Biol. Chem., 281, 32550-32558.
Rennecke, J., Rehberger, P. A., Furstenberger, G., Johannes, F. J., Stohr, M., Marks, F. & Richter, K. H. (1999). Protein kinase Cμ expression correlates with enhanced keratinocyte proliferation in normal and neoplastic mouse epidermis and in cell culture. Int J. Cancer 80, 98-103.
Ristich V L, Bowman P H, Dodd M E, Bollag W B. (2006) Protein kinase D distribution in normal human epidermis, basal cell carcinoma and psoriasis. Br J. Dermatol. 154, 586-593.
Singh P, Lillywhite B, Bannaghan C, Broad P., 2005, “Using IMAP technology to identify kinase inhibitors: comparison with a substrate depletion approach and analysis of the nature of false positives,” Comb Chem High Throughput Screen. Vol. 8, No. 4, pp. 319-25.
Stewart J R and O'Brian C A (2004). Resveratrol antagonizes EGFR-dependent Erk1/2 activation in androgen-independent prostate cancer cells with associated isozyme-selective PKCα inhibition. Invest. New Drugs 22, 107-117.
Storz P, Doppler H, Toker, A (2005), Protein kinase D mediates mitochondrion-to-nucleus signaling and detoxification from mitochondrial reactive oxygen species. Mol Cell Biol 25(19):8520-8530.
Storz P, Doppler H and Toker A (2004a). Protein kinase Cδ selectively regulates protein kinase D-dependent activation of NfkappaB in oxidative stress signaling. Mol. Cell. Biol. 24, 2614-2626.
Storz P, Doppler H, Toker A. (2004b). Activation loop phosphorylation controls protein kinase D-dependent activation of nuclear factor kappaB. Mol Pharmacol Oct; 66(4):870-9.
Storz, P and Toker, A (2003). Protein kinase D mediates a stress-induced NF-κB activation and survival pathway. EMBO J. 22, 109-120.
Trauzold A, Schmiedel S, Sipos B, Wermann H, Westphal S, Roder C, Klapper W, Arlt A, Lehnert L, Ungefroren H, Johannes F J, Kalthoff H. (2003) PKCmu prevents CD95-mediated apoptosis and enhances proliferation in pancreatic tumour cells. Oncogene 22(55):8939-8947
Van Lint, J., Rykx, A., Maeda, Y., Vantus, T., Sturany, S., Malhotra, V., Vandenheede, J. R. & Seufferlein, T. (2002). Protein Kinase D: an intracellular traffic regulator on the move. Trends Cell Biol. 12(4), 193-200.
Van Lint, J. V., Sinnett-Smith, J., & Rozengurt, E. (1995). Expression and characterization of PKD, a phorbol ester and diacylglycerol-stimulated serine protein kinase. J Biol Chem. 270(3), 1455-61.
Vega R B, Harrison B C, Meadows E, Roberts C R, Papst P J, Olson E N and McKinsey T A (2004). Protein kinases C and D mediate agonist-dependent cardiac hypertrophy through nuclear export of histone deacetylase 5. Mol. Cell. Biol 24, 8374-8385.
Wang Y, Waldron R T, Dhaka A, Patel A, Riley M M, Rozengurt E, Colicelli J. (2002). The RAS effector RINI directly competes with RAF and is regulated by 14-3-3 proteins. Mol Cell Biol. 22, 916-226.
Wong C, Jin Z G (2005). Protein kinase C-dependent protein kinase D activation modulates ERK signal pathway and endothelial cell proliferation by vascular endothelial growth factor. J Biol Chem. September 30; 280(39):33262-9.
Wood C D, Marklund U, Cantrell D A (2005). Dual phospholipase C/diacylglycerol requirement for protein kinase D1 activation in lymphocytes. J Biol Chem. February 18; 280(7):6245-51.
Zugaza, J. L., Sinnett-Smith, J., Van Lint, J. & Rozengurt, E. (1996). Protein Kinase D (PKD) activation in intact cells through a protein kinase C-dependent signal transduction pathway. EMBO J. 15, 6220-30.
Zugaza, J. L., Waldron, R. T., Sinnett-Smith, J. & Rozengurt, E.(1997). Bombesin, vasopressin, endothelin, bradykinin, and platelet-derived growth factor rapidly activate protein kinase D through a protein kinase C-dependent signal transduction pathway. J Biol Chem. 272(38), 23952-22960.

1-101. (canceled)

102. A compound selected from compounds of the following formula and pharmaceutically acceptable salts thereof: 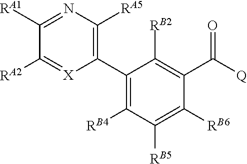
wherein:
X is independently C(R^{A3}) or N;
R^{A1} is independently —H or —NR^{NA11}R^{NA12};
wherein:
each R^{NA11} is independently —H or R^{Z1};
each R^{NA12} is independently —H or R^{Z1};
wherein:
each R^{Z1} is independently C₁₋₃alkyl or cyclopropyl;
and wherein additionally, each —NR^{NA11}R^{NA12} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{A3}, R^{A5}, R^{B2}, R^{B4}, R^{B5}, and R^{B6} is independently selected from:
—H, R^{Z2}, —F, —Cl, —Br, —OH, —OR^{Z2}, —SR^{Z2}, —CF₃, —OCF₃, —OCF₃, —CN, —NR^{NZ1}R^{NZ2}, —C(═O)—NR^{NZ1}R^{NZ2}, and —NR^{NZ3}C(═O)R^{Z2};
wherein:
each R^{NZ1} is independently —H or R^{Z2};
each R^{NZ2} is independently —H or R^{Z2};
each R^{NZ3} is independently —H or R^{Z2};
wherein:
each R^{Z2} is independently C₁₋₃alkyl or cyclopropyl;
and wherein additionally each —NR^{NZ1}R^{NZ2} may be azetidino, pyrrolidino, imidazolidino, N—(C₁₋₃ alkyl)-imidazolidino, pyrazolidino, N—(C₁₋₃alkyl)-pyrazolidino, piperidino, N—(C₁₋₃ alkyl)-piperidino, piperizino, morpholino, azepino, diazepino, or N—(C₁₋₃alkyl)-diazepino, each of which is optionally substituted with one or more C₁₋₃ alkyl groups;
Q is independently —NH₂, —NR^{NQ1}R^{NQ2}, or —W;
wherein:
R^{NQ1} is independently C₁₋₄alkyl;
R^{NQ2} is independently —H or C₁₋₄alkyl;
and additionally, —NR^{NQ1}R^{NQ2} may be azetidino, pyrrolidino, imidazolidino, N—(C₁₋₃ alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃ alkyl groups;
W is the following group: 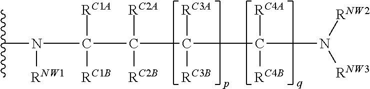
wherein:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃ alkyl)-imidazolidino, pyrazolidino, N—(C₁₋₃alkyl)-pyrazolidino, piperidino, N—(C₁₋₃ alkyl)-piperidino, piperizino, morpholino, azepino, diazepino, or N—(C₁₋₃alkyl)-diazepino, each of which is optionally substituted with one or more C₁₋₃ alkyl groups;
each of R^{C1A}, R^{C1B}, R^{C2A}, and R^{C2B} is independently —H or C₁₋₃alkyl;
each of R^{C3A} and R^{C3B} is independently —H or C₁₋₃alkyl; and
each of R^{C4A} and R^{C4B} is independently —H or C₁₋₃alkyl;
and additionally:
if p is 0 and q is 0, then:
(a1) R^{NW1} and one of R^{NW2} and R^{NW3} may together form:
—(CH₂)₂— or —(CH₂)₃—; or
(a2) one of R^{C1A} and R^{C1B} and one of R^{NW2} and R^{NW3} may together form:
—(CH₂)₃— or —(CH₂)₄—; or
(a3) one of R^{C2A} and R^{C2B} and one of R^{NW2} and R^{NW3} may together form:
—(CH₂)₄— or —(CH₂)₅—;
if p is 1 and q is 0, then:
(b1) R^{NW1} and one of R^{NW2} and R^{NW3} may together form:
—CH₂— or —(CH₂)₂—; or
(b2) one of R^{C1A} and R^{C1B} and one of R^{NW2} and R^{NW3} may together form:
—(CH₂)₂— or —(CH₂)₃—; or
(b3) one of R^{C2A} and R^{C2B} and one of R^{NW2} and R^{NW3} may together form:
—(CH₂)₃— or —(CH₂)₄—;
(b4) one of R^{C3A} and R^{C3B} and one of R^{NW2} and R^{NW3} may together form:
—(CH₂)₄— or —(CH₂)₅—; and
if p is 1 and q is 1, then:
(c1) R^{NW1} and one of R^{NW2} and R^{NW3} may together form:
—CH₂—; or
(c2) one of R^{C1A} and R^{C1B} and one of R^{NW2} and R^{NW3} may together form:
—CH₂— or —(CH₂)₂—; or
(c3) one of R^{C2A} and R^{C2B} and one of R^{NW2} and R^{NW3} may together form:
—(CH₂)₂— or —(CH₂)₃—; or
(c4) one of R^{C3A} and R^{C3B} and one of R^{NW2} and R^{NW3} may together form:
—(CH₂)₃— or —(CH₂)₄—; or
(c5) one of R^{C4A} and R^{C4B} and one of R^{NW2} and R^{NW3} may together form:
—(CH₂)₄— or —(CH₂)₅—;
R^{A2} is independently C₆₋₁₀-carboaryl or C₅₋₁₄heteroaryl; and is independently unsubstituted or substituted;
with the proviso is that the compound is not:
(B1) N-(3-dimethylamino-propyl)-3-[6-(3-methoxy-phenyl)-pyrazin-2-yl]-benzamide;
(B2) N-(2-dimethylamino-ethyl)-3-[6-(2-methoxy-phenyl)-pyrazin-2-yl]-benzamide;
(B3) N-(2-dimethylamino-ethyl)-3-[6-(3,4,5-trimethoxy-phenyl)-pyrazin-2-yl]-benzamide;
(B4) N-(3-dimethylamino-propyl)-3-[6-(4-hydroxy-phenyl)-pyrazin-2-yl]-benzamide;
(B5) N-(2-dimethylamino-ethyl)-3-[6-(4-hydroxymethyl-phenyl)-pyrazin-2-yl]-benzamide;
(B6) 3-[6-(3-acetylamino-phenyl)-pyrazin-2-yl]-N-(3-dimethylamino-propyl)-benzamide;
(B7) N-(2-dimethylamino-ethyl)-3-[6-(4-hydroxy-3-methoxy-phenyl)-pyrazin-2-yl]-benzamide;
(B8) 3-[6-amino-5-(4-hydroxy-3-methoxy-phenyl)-pyridin-3-yl]-benzamide;
(B9) 3-[6-amino-5-(2-methoxy-phenyl)-pyridin-3-yl]-benzamide; or
(B10) {3-[6-amino-5-(2-methoxy-phenyl)-pyridin-3-yl]-phenyl}-(4-methyl-piperazin-1-yl)-methanone.

103. A compound according to claim 102, wherein X is independently CH.

104. A compound according to claim 102, wherein X is independently N.

105. A compound according to claim 102, wherein R^{A1} is independently —H, —NH₂, —NHMe, —NMe₂, —NHEt, —NEt₂, or —NMeEt.

106. A compound according to claim 102, wherein R^{A1} is independently —NH₂.

107. A compound according to claim 102, wherein each of R^{A3}, R^{A5}, R^{B2}, R^{B4}, R^{B5}, and R^{B6} is independently selected from: —H, -Me, —F, —Cl, —Br, —OH, —OMe, —SMe, —CF₃, —OCF₃, —CN, —NH₂, —NHMe, —NMe₂, —C(═O)NH₂, —C(═O)NHMe, —C(═O)NMe₂, —NHC(═O)Me and —NMeC(═O)Me.

108. A compound according to claim 102, wherein each of R^{A3}, R^{A5}, R^{B2}, R^{B4}, R^{B5}, and R^{B6} is independently —H.

109. A compound according to claim 102, wherein Q is independently —W.

110. A compound according to claim 102, wherein:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H or C₁₋₃alkyl;
each of R^{C3A} and R^{C3B} is independently —H or C₁₋₃alkyl; and
each of R^{C4A} and R^{C4B} is independently —H or C₁₋₃alkyl;
and W may additionally be selected from:

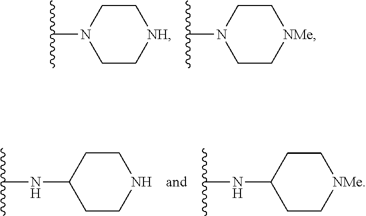

111. A compound according to claim 102, wherein:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H or C₁₋₃alkyl;
each of R^{C3A} and R^{C3B} is independently —H or C₁₋₃alkyl; and
each of R^{C4A} and R^{C4B} is independently —H or C₁₋₃alkyl.

112. A compound according to claim 102, wherein:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
each of R^{C1A}, R^{C1B}, R^{C2A}, and R^{C2B} is independently —H or C₁₋₃alkyl;
each of R^{C3A} and R^{C3B} is independently —H or C₁₋₃alkyl; and
each of R^{C4A} and R^{C4B} is independently —H or C₁₋₃alkyl.

113. A compound according to claim 112, wherein R^{NW1} is independently —H.

114. A compound according to claim 112, wherein:
each of R^{C1A}, R^{C1B}, R^{C2A}, and R^{C2B} is independently —H or -Me;
each of R^{C3A} and R^{C3B} is independently —H or -Me; and
each of R^{C4A} and R^{C4B} is independently —H or -Me.

115. A compound according to claim 112, wherein each of R^{NW2} and R^{NW3} is independently C₁₋₄alkyl.

116. A compound according to claim 112, wherein each of R^{NW2} and R^{NW3} is independently -Me.

117. A compound according to claim 112, wherein:
p is 0 and q is 0; or
p is 1 and q is O.

118. A compound according to claim 112, wherein:
p is 0 and q is 0.

119. A compound according to claim 112, wherein:
p is 1 and q is O.

120. A compound according to claim 102, wherein:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₄alkyl;
and additionally: —NR^{NW2}R^{NW3} may be azetidino, pyrrolidino, imidazolidino, N—(C₁₋₃alkyl)-imidazolidino, pyrazolidino, N—(C₁₋₃alkyl)-pyrazolidino, piperidino, N—(C₁₋₃alkyl)-piperidino, piperizino, morpholino, azepino, diazepino, or N—(C₁₋₃alkyl)-diazepino, each of which is optionally substituted with one or more C₁₋₃alkyl groups;
each of R^{C1A}, R^{C1B}, R^{C2A}, and R^{C2B} is independently —H or C₁₋₃alkyl;
each of R^{C3A} and R^{C3B} is independently —H or C₁₋₃alkyl; and
each of R^{C4A} and R^{C4B} is independently —H or C₁₋₃alkyl.

121. A compound according to claim 102, wherein:
p is 0 and q is 0; or
p is 1 and q is 0; or
p is 1 and q is 1;
R^{NW1} is independently —H or C₁₋₃alkyl;
each of R^{NW2} and R^{NW3} is independently —H or C₁₋₃alkyl;
each of R^{C1A}, R^{C1B}, R^{C2A}, and R^{C2B} is independently —H;
each of R^{C3A} and R^{C3B} is independently —H; and
each of R^{C4A} and R^{C4B} is independently —H.

122. A compound according to claim 102, wherein:
p is 0 and q is 0; or
p is 1 and q is 0; or
p is 1 and q is 1;
R^{NW1} is independently —H;
each of R^{NW2} and R^{NW3} is independently —H, -Me, or -Et;
each of R^{C1A}, R^{C1B}, R^{C2A}, and R^{C2B} is independently —H;
each of R^{C3A} and R^{C3B} is independently —H; and
each of R^{C4A} and R^{C4B} is independently —H.

123. A compound according to claim 102, wherein:
p is 0 and q is 0; or
p is 1 and q is 0; or
p is 1 and q is 1;
R^{NW1} is independently —H;
each of R^{NW2} and R^{NW3} is independently -Me;
each of R^{C1A}, R^{C1B}, R^{C2A}, and R^{C2B} is independently —H;
each of R^{C3A} and R^{C3B} is independently —H; and
each of R^{C4A} and R^{C4B} is independently —H.

124. A compound according claim 102, wherein W is the following group:

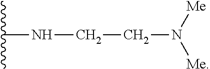

125. A compound according to claim 102, wherein R^{A2} is independently: phenyl, pyridyl, pyrimidinyl, pyrazinyl, pyridazinyl, furanyl, thienyl, pyrrolyl, thiazolyl, oxazolyl, isothiazolyl, isoxazolyl, benzofuranyl, benzo[b]thienyl, indolyl, benzo[1,3]dioxolyl, naphthyl, quinolinyl, isoquinolinyl, quinoxalinyl, indazolyl, 2,3-dihydrobenzo[1,4]dioxinyl, dihydrobenzofuranyl, dibenzofuranyl, and dibenzothienyl; and is independently unsubstituted or substituted.

126. A compound according to claim 102, wherein R^{A2} is independently: phenyl, pyridyl, pyrazinyl, pyrimidinyl, or pyridazinyl, naphthyl, quinolinyl, isoquinolinyl, quinoxalinyl, or indazolyl; and is independently unsubstituted or substituted.

127. A compound according to claim 102, wherein R^{A2} is independently phenyl, and is independently unsubstituted or substituted.

128. A compound according to claim 102, wherein R^{A2} is independently pyridyl, and is independently unsubstituted or substituted.

129. A compound according to claim 102, wherein R^{A2} is independently naphthyl, and is independently unsubstituted or substituted.

130. A compound according to claim 102, wherein R^{A2} is independently unsubstituted or substituted with one or more substituents independently selected from:
—F, —Cl, —Br, —I,
—CN, —CH₂CN,
—R^{aa}, —CF₃,
—Ph, —CH₂Ph, thienyl,
—OH, —R^{L}—OH, —R^{L}—OR^{aa},
—OR^{aa}, —OCF₃,
—OPh, —OCH₂Ph,
—O—R^{L}—OH, —O—R^{L}—OR^{aa},
—SR^{aa}, —SPh,
—SO₂R^{aa}, SO₂Ph,
—NHSO₂R^{aa}, NHSO₂Ph,
—NH₂, —NHR^{aa}, —N(R^{aa})₂,
—NHPh, —NHCH₂Ph,
—NH—R^{L}—NH₂, —NH—R^{L}—NHR^{aa}, —NH—R^{L}—N(R^{aa})₂,
—C(═O)NH₂, —C(═O)NHR^{aa}, —C(═O)N(R^{aa})₂,
—C(═O)NHPh, —C(═O)NHCH₂Ph,
—NHC(═O)R^{aa},
—NHC(═O)Ph, —NHC(═O)CH₂Ph,
—C(═O)OH, —C(═O)OR^{aa},
—C(═O)OPh, and —C(═O)OCH₂Ph,
—C(═O)Ph;
wherein:
each Ph is independently phenyl, optionally substituted with 1 to 4 groups selected from:
—F, —Cl, —Br, —I,
—CN,
—R^{aa}, —CF₃,
—OH, —OR^{aa},
—O—R^{L}—OH, —O—R^{L}—OR^{aa},
—OCF₃,
—NH₂, —NHR^{aa}, —N(R^{aa})₂,
—C(═O)NH₂, —C(═O)NHR^{aa}, —C(═O)N(R^{aa})₂,
—NHC(═O)R^{aa},
and wherein:
each R^{aa} is independently C₁₋₄alkyl;
additionally, for each —N(R^{aa})₂, two R^{aa} groups, taken together with the nitrogen atom to which they are attached, may form a non-aromatic heterocyclic ring having from 4 to 7 ring atoms, optionally substituted with one or more C₁₋₃alkyl groups; and
each R^{L} is independently C₁₋₄alkylenyl.

131. A compound according to claim 102, selected from the following compounds and pharmaceutically acceptable salts thereof:
(X-008) [3-(5-Amino-6-dibenzofuran-4-yl-pyrazin-2-yl)-phenyl]-(4-methyl-piperazin-1-yl)-methanone;
(X-009) {3-[6-(4-Hydroxy-3-methoxy-phenyl)-pyrazin-2-yl]-phenyl}-morpholin-4-yl-methanone;
(X-010) {3-[6-(4-Hydroxy-3-methoxy-phenyl)-pyrazin-2-yl]-phenyl}-piperazin-1-yl-methanone;
(X-011) {3-[6-(4-Hydroxy-3-methoxy-phenyl)-pyrazin-2-yl]-phenyl}-pyrrolidin-1-yl-methanone;
(X-012) 3-(5-Amino-6-dibenzofuran-4-yl-pyrazin-2-yl)-N-(2-dimethylamino-ethyl)-benzamide;
(X-013) 3-(6-Dibenzofuran-4-yl-pyrazin-2-yl)-N-(2-dimethylamino-ethyl)-benzamide;
(X-014) 3-(6-Quinolin-5-yl-pyrazin-2-yl)-benzamide;
(X-015) 3-[5-Amino-6-(2-methoxy-phenyl)-pyrazin-2-yl]-N-(2-dimethylamino-ethyl)-benzamide;
(X-016) 3-[5-Amino-6-(4-fluoro-3-methoxy-phenyl)-pyrazin-2-yl]-N-(2-dimethylamino-ethyl)-benzamide;
(X-017) 3-[5-Amino-6-(4-hydroxy-3-methoxy-phenyl)-pyrazin-2-yl]-N-(2-dimethylamino-ethyl)-benzamide;
(X-018) 3-[6-(4-Hydroxy-3-methoxy-phenyl)-pyrazin-2-yl]-benzamide;
(X-019) 3-[6-(4-Hydroxy-3-methoxy-phenyl)-pyrazin-2-yl]-N-(1-methyl-piperidin-4-yl)-benzamide;
(X-020) 3-[6-(4-Hydroxy-3-methoxy-phenyl)-pyrazin-2-yl]-N-isopropyl-benzamide;
(X-021) N-(2-Amino-ethyl)-3-[6-(4-hydroxy-3-methoxy-phenyl)-pyrazin-2-yl]-benzamide;
(X-022) N-(2-Diethylamino-ethyl)-3-[6-(4-hydroxy-3-methoxy-phenyl)-pyrazin-2-yl]-benzamide;
(X-023) 3-[5-Amino-6-(6-hydroxy-naphthalen-2-yl)-pyrazin-2-yl]-N-(2-dimethylamino-ethyl)-benzamide;
(X-024) 3-[5-Amino-6-(2-methoxy-pyridin-4-yl)-pyrazin-2-yl]-N-(2-dimethylamino-ethyl)-benzamide;
(X-025) 3-[5-Amino-6-(6-methoxy-naphthalen-2-yl)-pyrazin-2-yl]-N-(2-dimethylamino-ethyl)-benzamide;
(X-026) 3-[5-Amino-6-(3-methoxy-phenyl)-pyrazin-2-yl]-N-(2-dimethylamino-ethyl)-benzamide;
(X-027) 3-[5-Amino-6-(3-methoxy-phenyl)-pyrazin-2-yl]-N-(2-diethylamino-ethyl)-benzamide;
(Y-004) {3-[6-Amino-5-(4-hydroxy-3-methoxy-phenyl)-pyridin-3-yl]-phenyl}-(4-methyl-piperazin-1-yl)-methanone;
(Y-005) 3-(2-Amino-2′-methoxy-[3,4]bipyridinyl-5-yl)-N-(2-dimethylamino-ethyl)-benzamide;
(Y-006) 3-(2-Amino-6′-chloro-[3,3]bipyridinyl-5-yl)-N-(2-dimethylamino-ethyl)-benzamide;
(Y-007) 3-(2-Amino-6′-hydroxy-[3,3]bipyridinyl-5-yl)-N-(2-dimethylamino-ethyl)-benzamide;
(Y-008) 3-(2-Amino-6′-methoxy-[3,3]bipyridinyl-5-yl)-N-(2-dimethylamino-ethyl)-benzamide;
(Y-009) 3-(6-Amino-5-benzo[1,3]dioxol-5-yl-pyridin-3-yl)-benzamide;
(Y-010) 3-(6-Amino-5-benzo[1,3]dioxol-5-yl-pyridin-3-yl)-N-(2-dimethylamino-ethyl)-benzamide;
(Y-011) 3-(6-Amino-5-benzo[b]thiophen-2-yl-pyridin-3-yl)-N-(2-dimethylamino-ethyl)-benzamide;
(Y-012) 3-(6-Amino-5-benzofuran-2-yl-pyridin-3-yl)-N-(2-dimethylamino-ethyl)-benzamide;
(Y-013) 3-(6-Amino-5-biphenyl-3-yl-pyridin-3-yl)-N-(2-dimethylamino-ethyl)-benzamide;
(Y-014) 3-(6-Amino-5-biphenyl-4-yl-pyridin-3-yl)-N-(2-dimethylamino-ethyl)-benzamide;
(Y-015) 3-(6-Amino-5-dibenzothiophen-4-yl-pyridin-3-yl)-N-(2-dimethylamino-ethyl)-benzamide;
(Y-016) 3-(6-Amino-5-furan-3-yl-pyridin-3-yl)-benzamide;
(Y-017) 3-(6-Amino-5-furan-3-yl-pyridin-3-yl)-N-(2-dimethylamino-ethyl)-benzamide;
(Y-018) 3-(6-Amino-5-isoquinolin-5-yl-pyridin-3-yl)-N-(2-dimethylamino-ethyl)-benzamide;
(Y-019) 3-(6-Amino-5-naphthalen-1-yl-pyridin-3-yl)-N-(2-dimethylamino-ethyl)-benzamide;
(Y-020) 3-(6-Amino-5-naphthalen-2-yl-pyridin-3-yl)-N-(2-dimethylamino-ethyl)-benzamide;
(Y-021) 3-(6-Amino-5-quinolin-5-yl-pyridin-3-yl)-benzamide;
(Y-022) 3-(6-Amino-5-quinolin-5-yl-pyridin-3-yl)-N-(2-dimethylamino-ethyl)-benzamide;
(Y-023) 3-(6-Amino-5-quinolin-8-yl-pyridin-3-yl)-N-(2-dimethylamino-ethyl)-benzamide;
(Y-024) 3-(6-Amino-5-thiophen-2-yl-pyridin-3-yl)-N-(2-dimethylamino-ethyl)-benzamide;
(Y-025) 3-[6-Amino-5-(1H-indol-5-yl)-pyridin-3-yl]-benzamide;
(Y-026) 3-[6-Amino-5-(1H-indol-5-yl)-pyridin-3-yl]-N-(2-dimethylamino-ethyl)-benzamide;
(Y-027) 3-[6-Amino-5-(1-methyl-1H-indol-5-yl)-pyridin-3-yl]-N-(2-dimethylamino-ethyl)-benzamide;
(Y-028) 3-[6-Amino-5-(2,3-dihydro-benzofuran-5-yl)-pyridin-3-yl]-N-(2-dimethylamino-ethyl)-benzamide;
(Y-029) 3-[6-Amino-5-(2,4-dimethoxy-pyrimidin-5-yl)-pyridin-3-yl]-N-(2-dimethylamino-ethyl)-benzamide;
(Y-030) 3-[6-Amino-5-(2-benzyloxy-phenyl)-pyridin-3-yl]-N-(2-dimethylamino-ethyl)-benzamide;
(Y-031) 3-[6-Amino-5-(2-fluoro-biphenyl-4-yl)-pyridin-3-yl]-N-(2-dimethylamino-ethyl)-benzamide;
(Y-032) 3-[6-Amino-5-(2-phenoxy-phenyl)-pyridin-3-yl]-N-(2-dimethylamino-ethyl)-benzamide;
(Y-033) 3-[6-Amino-5-(3,4-difluoro-phenyl)-pyridin-3-yl]-N-(2-dimethylamino-ethyl)-benzamide;
(Y-034) 3-[6-Amino-5-(3,4-dimethoxy-phenyl)-pyridin-3-yl]-benzamide;
(Y-035) 3-[6-Amino-5-(3,4-dimethoxy-phenyl)-pyridin-3-yl]-N-(2-dimethylamino-ethyl)-benzamide;
(Y-036) 3-[6-Amino-5-(3,4-dimethyl-phenyl)-pyridin-3-yl]-N-(2-dimethylamino-ethyl)-benzamide;
(Y-037) 3-[6-Amino-5-(3,5-dichloro-phenyl)-pyridin-3-yl]-N-(2-dimethylamino-ethyl)-benzamide;
(Y-038) 3-[6-Amino-5-(3-benzyloxy-phenyl)-pyridin-3-yl]-N-(2-dimethylamino-ethyl)-benzamide;
(Y-039) 3-[6-Amino-5-(3-chloro-4-fluoro-phenyl)-pyridin-3-yl]-N-(2-dimethylamino-ethyl)-benzamide;
(Y-040) 3-[6-Amino-5-(3-chloro-phenyl)-pyridin-3-yl]-N-(2-dimethylamino-ethyl)-benzamide;
(Y-041) 3-[6-Amino-5-(3-cyano-phenyl)-pyridin-3-yl]-N-(2-dimethylamino-ethyl)-benzamide;
(Y-042) 3-[6-Amino-5-(3-fluoro-4-hydroxy-phenyl)-pyridin-3-yl]-N-(2-dimethylamino-ethyl)-benzamide;
(Y-043) 3-[6-Amino-5-(3-fluoro-4-methoxy-phenyl)-pyridin-3-yl]-N-(2-dimethylamino-ethyl)-benzamide;
(Y-044) 3-[6-Amino-5-(3-fluoro-4-methyl-phenyl)-pyridin-3-yl]-N-(2-dimethylamino-ethyl)-benzamide;
(Y-045) 3-[6-Amino-5-(3-fluoro-biphenyl-4-yl)-pyridin-3-yl]-N-(2-dimethylamino-ethyl)-benzamide;
(Y-046) 3-[6-Amino-5-(3-isopropoxy-phenyl)-pyridin-3-yl]-N-(2-dimethylamino-ethyl)-benzamide;
(Y-047) 3-[6-Amino-5-(3-methoxymethyl-phenyl)-pyridin-3-yl]-N-(2-dimethylamino-ethyl)-benzamide;
(Y-048) 3-[6-Amino-5-(3-methoxy-phenyl)-pyridin-3-yl]-N-(2-dimethylamino-ethyl)-benzamide;
(Y-049) 3-[6-Amino-5-(3-phenoxy-phenyl)-pyridin-3-yl]-N-(2-dimethylamino-ethyl)-benzamide;
(Y-050) 3-[6-Amino-5-(3-trifluoromethoxy-phenyl)-pyridin-3-yl]-N-(2-dimethylamino-ethyl)-benzamide;
(Y-051) 3-[6-Amino-5-(4-benzyloxy-phenyl)-pyridin-3-yl]-N-(2-dimethylamino-ethyl)-benzamide;
(Y-052) 3-[6-Amino-5-(4-chloro-3-fluoro-phenyl)-pyridin-3-yl]-N-(2-dimethylamino-ethyl)-benzamide;
(Y-053) 3-[6-Amino-5-(4-ethylsulfanyl-phenyl)-pyridin-3-yl]-N-(2-dimethylamino-ethyl)-benzamide;
(Y-054) 3-[6-Amino-5-(4-fluoro-2-methoxy-phenyl)-pyridin-3-yl]-N-(2-dimethylamino-ethyl)-benzamide;
(Y-055) 3-[6-Amino-5-(4-fluoro-3-methoxy-phenyl)-pyridin-3-yl]-benzamide;
(Y-056) 3-[6-Amino-5-(4-fluoro-3-methoxy-phenyl)-pyridin-3-yl]-N-(2-dimethylamino-ethyl)-benzamide;
(Y-057) 3-[6-Amino-5-(4-fluoro-3-trifluoromethyl-phenyl)-pyridin-3-yl]-N-(2-dimethylamino-ethyl)-benzamide;
(Y-058) 3-[6-Amino-5-(4-fluoro-phenyl)-pyridin-3-yl]-N-(2-dimethylamino-ethyl)-benzamide;
(Y-059) 3-[6-Amino-5-(4-hydroxy-3-methoxy-phenyl)-pyridin-3-yl]-N-(2-dimethylamino-ethyl)-benzamide;
(Y-060) 3-[6-Amino-5-(4-hydroxy-3-methoxy-phenyl)-pyridin-3-yl]-N-(3-dimethylamino-propyl)-benzamide;
(Y-061) 3-[6-Amino-5-(4-hydroxymethyl-phenyl)-pyridin-3-yl]-N-(2-dimethylamino-ethyl)-benzamide;
(Y-062) 3-[6-Amino-5-(4-isobutyl-phenyl)-pyridin-3-yl]-N-(2-dimethylamino-ethyl)-benzamide;
(Y-063) 3-[6-Amino-5-(4-methoxy-phenyl)-pyridin-3-yl]-N-(2-dimethylamino-ethyl)-benzamide;
(Y-064) 3-[6-Amino-5-(4-trifluoromethoxy-phenyl)-pyridin-3-yl]-N-(2-dimethylamino-ethyl)-benzamide;
(Y-065) 3-{6-Amino-5-[4-(tetrahydro-pyran-2-yloxy)-phenyl]-pyridin-3-yl}-N-(2-dimethylamino-ethyl)-benzamide;
(Y-066) 4-{2-Amino-5-[3-(2-dimethylamino-ethylcarbamoyl)-phenyl]-pyridin-3-yl}-2-methoxy-benzoic acid methyl ester;
(Y-067) 3-[6-Amino-5-(4-phenoxy-phenyl)-pyridin-3-yl]-N-(2-dimethylamino-ethyl)-benzamide;
(Y-068) 3-[6-Amino-5-(4-tert-butyl-phenyl)-pyridin-3-yl]-N-(2-dimethylamino-ethyl)-benzamide;
(Y-069) 3-[6-Amino-5-(4-trifluoromethyl-phenyl)-pyridin-3-yl]-N-(2-dimethylamino-ethyl)-benzamide;
(Y-070) 3-[6-Amino-5-(4-benzoyl-phenyl)-pyridin-3-yl]-N-(2-dimethylamino-ethyl)-benzamide;
(Y-071) 3-(2-Amino-2′-hydroxy-[3,4]bipyridinyl-5-yl)-N-(2-dimethylamino-ethyl)-benzamide;
(Y-072) 3-[6-Amino-5-(3-methanesulfonyl-phenyl)-pyridin-3-yl]-N-(2-dimethylamino-ethyl)-benzamide;
(Y-073) 3-[6-Amino-5-(3-ethoxy-phenyl)-pyridin-3-yl]-N-(2-dimethylamino-ethyl)-benzamide;
(Y-074) N-(2-Dimethylamino-ethyl)-3-[5-(3-methoxy-phenyl)-6-methylamino-pyridin-3-yl]-benzamide;
(Y-075) 3-[6-Amino-5-(3-methoxy-phenyl)-pyridin-3-yl]-N-(2-dimethylamino-ethyl)-N-methyl-benzamide;
(Y-076) 3-[5-(3-Acetylamino-phenyl)-6-amino-pyridin-3-yl]-N-(2-dimethylamino-ethyl)-benzamide;
(Y-077) 3-[6-Amino-5-(3-hydroxy-phenyl)-pyridin-3-yl]-N-(2-dimethylamino-ethyl)-benzamide;
(Y-078) 3-(6-Amino-5-isoquinolin-4-yl-pyridin-3-yl)-N-(2-dimethylamino-ethyl)-benzamide;
(Y-079) 3-[6-Amino-5-(4-hydroxy-phenyl)-pyridin-3-yl]-N-(2-dimethylamino-ethyl)-benzamide;
(Y-080) 3-[6-Amino-5-(3-cyanomethyl-phenyl)-pyridin-3-yl]-N-(2-dimethylamino-ethyl)-benzamide;
(Y-081) 3-[6-Amino-5-(3,5-dimethoxy-phenyl)-pyridin-3-yl]-N-(2-dimethylamino-ethyl)-benzamide;
(Y-082) 3-[6-Amino-5-(6-hydroxy-naphthalen-2-yl)-pyridin-3-yl]-N-(2-dimethylamino-ethyl)-benzamide;
(Y-083) 3-(2′-Amino-6-methoxy-[2,3]bipyridinyl-5′-yl)-N-(2-dimethylamino-ethyl)-benzamide;
(Y-084) 3-(2-Amino-[3,4]bipyridinyl-5-yl)-N-(2-dimethylamino-ethyl)-benzamide;
(Y-085) 3-(2-Amino-2′-chloro-[3,4]bipyridinyl-5-yl)-N-(2-dimethylamino-ethyl)-benzamide;
(Y-086) 3-[6-Amino-5-(2-methylsulfanyl-pyrimidin-4-yl)-pyridin-3-yl]-N-(2-dimethylamino-ethyl)-benzamide;
(Y-087) 3-[6-Amino-5-(6-methoxy-naphthalen-2-yl)-pyridin-3-yl]-N-(2-dimethylamino-ethyl)-benzamide;
(Y-088) 3-[6-Amino-5-(3,4,5-trimethoxy-phenyl)-pyridin-3-yl]-N-(2-dimethylamino-ethyl)-benzamide;
(Y-089) 3-(2-Amino-5′-methoxy-[3,3]bipyridinyl-5-yl)-N-(2-dimethylamino-ethyl)-benzamide;
(Y-090) 3-(6-Amino-5-quinolin-3-yl-pyridin-3-yl)-N-(2-dimethylamino-ethyl)-benzamide;
(Y-091) 3-(6-Amino-5-quinoxalin-6-yl-pyridin-3-yl)-N-(2-dimethylamino-ethyl)-benzamide;
(Y-092) 3-[6-Amino-5-(3-methoxy-phenyl)-pyridin-3-yl]-N-(2-dimethylamino-1-methyl-ethyl)-benzamide;
(Y-093) 3-[6-Amino-5-(6-methoxy-pyrimidin-4-yl)-pyridin-3-yl]-N-(2-dimethylamino-ethyl)-benzamide;
(Y-094) 3-[6-Amino-5-(1H-indazol-5-yl)-pyridin-3-yl]-N-(2-dimethylamino-ethyl)-benzamide;
(Y-095) 3-[6-Amino-5-(6-ethoxy-naphthalen-2-yl)-pyridin-3-yl]-N-(2-dimethylamino-ethyl)-benzamide, and
(Y-096) 3-[6-Amino-5-(3-methoxy-phenyl)-pyridin-3-yl]-N-(2-diethylamino-ethyl)-benzamide.

132. A pharmaceutical composition comprising a compound according to claim 102, and a pharmaceutically acceptable carrier or diluent.

133. A method of preparing a pharmaceutical composition comprising the step of admixing a compound according to claim 102 and a pharmaceutically acceptable carrier or diluent.

134. A method of inhibiting PKD in a cell, in vitro, comprising contacting the cell with an effective amount of a compound as defined in claim 102, without the recited proviso regarding compounds (B1) to (B10).

135. A method of treatment of:
a disease or condition that is mediated by PKD;
a disease or condition that is ameliorated by the inhibition of PKD;
a proliferative condition;
cancer;
a hyperproliferative skin disorder; psoriasis; actinic keratosis; non-melanoma skin cancer;
a disease or condition that is characterised by inappropriate, excessive, and/or undesirable angiogenesis;
an inflammatory disease; or
a disease or disorder associated with heart remodelling, myocyte hypertrophy of the heart, impaired contractility of the heart, pump failure of the heart, pathologic cardiac hypertrophy, and/or heart failure;
comprising administering to a subject in need of treatment a therapeutically-effective amount of a compound as defined in claim 102, without the recited proviso regarding compounds (B1) to (B10).
